(19) United States
(12) Patent Application Publication
(10) Pub. No.: US 2022/0307046 A1
(43) Pub. Date: Sep. 29, 2022

(54) PLANT CELL MATRICES AND METHODS THEREOF

(71) Applicant: Calyxt, Inc., Rosewille, MN (US)

(72) Inventors: Benjamin Campbell, Saint Paul, MN (US); Lauren Harrison, Minneapolis, MN (US); Rachelle A. Lapham, Ramsey, MN (US); Jonathan Mayers, Saint Paul, MN (US); Anders Uppgaard, Minneapolis, MN (US)

(73) Assignee: Calyxt, Inc., Roseville, MN (US)

(21) Appl. No.: 17/726,141

(22) Filed: Apr. 21, 2022

Related U.S. Application Data
(63) Continuation of application No. PCT/US2022/021014, filed on Mar. 18, 2022.
(60) Provisional application No. 63/162,702, filed on Mar. 18, 2021.
(60) Provisional application No. 63/184,487, filed on May 5, 2021.
(60) Provisional application No. 63/240,660, filed on Sep. 3, 2021.
(60) Provisional application No. 63/304,850, filed on Jan. 31, 2022.

(51) Int. Cl. C12N 15/82; C12N 5/04; C12N 5/00; C12N 1/20; C12N 15/62; C12N 15/74; C12N 15/52; C12P 21/02; C07K 14/77; C12P 19/60

(52) U.S. Cl. CPC C12N 15/8257; C12N 5/04; C12N 5/0025; C12N 1/20; C12N 15/62; C12N 15/743; C12N 15/52; C12P 21/02; C07K 14/77; C12P 19/60; C12N 2800/101

(57) ABSTRACT

Example embodiments in accordance with the present disclosure are directed to methods comprising contacting a plant part with a nucleotide sequence encoding a gene that induces plant cell matrix (PCM) formation, and culturing the plant part under growth conditions to enhance PCM formation.

Incorporated by reference in its entirety is a computer-readable nucleotide/amino acid sequence listing, an ASCII text file which is 299 kb in size, submitted concurrently herewith, and identified as follows: “C1633110111_SequenceListing_ST25” and created on Mar. 17, 2022.

### BACKGROUND

Biomass and compounds can be used in a wide variety of fields. For example, biomass and/or compounds generated from biomass tissue in fields such as cosmeceuticals, nutraceuticals, pharmaceuticals, advanced materials, and in food applications.

### SUMMARY

This summary is provided to introduce a selection of concepts in a simplified form that are further described below in the Detailed Description.

In one aspect, the disclosure provides a method of transforming a plant part to induce formation of a collection of plant cells, referred to herein as “a plant cell matrix (PCM)”, and/or production of a PCM-derived compound.

Various aspects are directed to a method comprising contacting a plant part with a nucleotide sequence encoding a gene that induces PCM formation, and culturing the plant part under growth conditions to enhance PCM formation.

In some aspects, contacting the plant part with the nucleotide sequence comprises contacting the plant part with a bacterium strain comprising the nucleotide sequence.

In some aspects, the growth conditions comprise conditions selected from (such as being selected from the group consisting of): a liquid culture medium, a type of culture medium, an amount of contact with the culture medium, a type of contact with the culture medium, a plant type, and a combination thereof.

In some aspects, contacting the plant part with the nucleotide sequence or bacterium strain and culturing of the plant part are performed under the growth conditions to enhance PCM formation, thereby resulting in production of PCM tissue at greater production level than tissue produced by a wild-type plant or a plant grown in the field.

In some aspects, production of PCM tissue in the PCM is at least about 2-fold to about 500-fold a production level as compared to production of tissue in wild-type plant or plant grown in the field.

In some aspects, the PCM comprises plant cells transformed by the contact with the nucleotide sequence and includes a plurality of different plant cells types, the plurality of different plant cells types comprising plant cells selected from: (such as being selected from the group consisting of) plant stem cells, maturing cells, mature cells, and a combination thereof.

In some aspects, the plant part is a seedling, a petiole, an internode, a node, a meristem, or a leaf

In some aspects the plant part is from a Cannabaceae plant, a Brassicaceae plant, a Solanaceae plant, a Fabaceae plant, or an Apiacea plant.

In some aspects, contacting the plant part and culturing the plant part induces production of a PCM-derived compound at a greater level than in tissue of a wild-type plant.

In some aspects, contacting the plant part with the nucleotide sequence and culturing the plant part comprises contacting the plant part with a Rhizobium or Agrobacterium strain comprising a root-inducing (Ri) plasmid or a tumor-inducing (Ti) plasmid and the nucleotide sequence encoding the gene that induces the PCM formation, and culturing the plant part to enhance the transformation and induce the PCM formation by at least 2-fold a production level as compared to tissue production of a wild-type plant.

In some aspects, culturing the plant part further induces the production of a PCM-derived compound by at least a 2-fold a production level as compared to production in tissue of a wild-type plant.

In some aspects, culturing the plant part under the growth conditions comprises intermittently contacting the plant part with a culture medium containing sugar and basal salt.

In some aspects, intermittently contacting the plant part with the culture medium comprises cycling between contacting the plant part with the culture medium and not contacting the plant part with the culture medium at a duty cycle of between about 1 percent and about 25 percent.

In some aspects, culturing the plant part induces production of a PCM-derived compound and the PCM-derived compound comprises a core precursor compound that is produced by the PCM at an increased production level as compared to production in tissue of a wild-type plant.

In some aspects, the core precursor compound comprises a compound selected from (such as being selected from the group consisting of): amino acids, organic acids, fatty acids, sugars, carbohydrates, phenolics, alkaloids, isoprenes, terpenes, sterols, fiber, cannabinoids, and a combination thereof.

In some aspects, the PCM produces a plurality of core precursor compounds each at an increased production level as compared to production in the tissue of the wild-type plant, the plurality of core precursor compounds comprising compounds selected from (such as being selected from the group consisting of): cannabisativine, anhydrocannabisativine, friedelin, epifriedelanol, beta-amyrin, beta-sitosterol, campesterol, stigmasterol, cellulose, tetrahydrocannabinol (THC), cannabigerolic acid (CBGA), cannabidiolic acid (CBDA), carvone, dihydrocarvone, p-hydroxy-trans-cinnamamide, lignans, choline, orientin, vitexin, isovitexin, quercetin, luteolin, kaempferol, apigenin, and a combination thereof.

In some aspects, the PCM produces a plurality of core precursor compounds each at an increased production level as compared to production in the tissue of the wild-type plant, the plurality of core precursor compounds comprising cannabisativine, anhydrocannabisativine, friedelin, epifriedelanol, beta-amyrin, beta-sitosterol, campesterol, and stigmasterol.

In some aspects, culturing the plant part induces production of a PCM-derived compound and the PCM-derived compound comprises a recombinant compound, and the method further comprises contacting the plant part with a nucleotide sequence encoding the recombinant compound.

In some aspects, the recombinant compound comprises a compound selected from (such as being selected from the group consisting of): a protein or production source for a metabolite selected from the group consisting of an allergen, vaccine, enzyme, enzyme inhibitor, antibody, antibody fragment, antigen, toxin, anti-microbial peptide, hormones, growth factor, blood protein, receptor, signaling protein, fusion or labelled protein, albumin, betalain, coagulation factor, immunoglobulin, transferrin, sulphatases, digestive enzyme, lipase, pepsin, trypsin, interleukin, sugar, interferon, and a combination thereof.

In some aspects, the method further comprises screening new growth from the cultured plant part for the PCM formation.

In some aspects, the plant part is cultured in a culture medium is selected from a liquid culture medium and a solid growth medium in the absence of added plant growth hormones, and optionally comprises a selection agent.

In some aspects, contacting the plant part with the nucleotide sequence comprises simultaneously introducing to the plant part: a first transgene associated with PCM formation, and a second transgene associated with a PCM-derived compound. And the method further comprises cultivating the plant part as transformed to generate PCM tissue, wherein the plant part is a seedling, a hypocotyl segment, a petiole, an internode, a node, a meristem, or a leaf.

In some aspects, contacting the plant part with the nucleotide sequence and culturing the plant part comprises contacting the plant part the nucleotide sequence encoding the gene that induces PCM formation, culturing the plant part to enhance PCM formation, contacting formed PCM tissue from the PCM with a nucleotide sequence encoding a PCM-derived compound, and culturing the PCM tissue to enhance production of the PCM-derived compound by the PCM.

Various aspects are directed to a PCM culture generated according to the method of claim 1 or 2.

Various aspects are directed to method comprising contacting a plant part with a bacterium strain containing a Ri plasmid or a Ti plasmid, a nucleotide sequence encoding a PCM-derived compound, and a nucleotide sequence encoding a gene that induces PCM formation, and culturing the plant part under infection and growth conditions to enhance transformation, induce the PCM formation, and induce production of the PCM-derived compound.

In some aspects, contacting and culturing the plant part comprises transforming the plant part with the bacterium strain, inducing formation of PCM tissue from the plant part as transformed, and culturing the PCM tissue in a culture medium under the growth conditions.

In some aspects, the bacterium strain comprises the Ri plasmid comprising the nucleotide sequence encoding the gene that induces PCM formation, and the nucleotide sequence encoding the PCM-derived compound.

In some aspects, the bacterium strain comprises the Ri plasmid, the nucleotide sequence encoding the gene that induces PCM formation, and the nucleotide sequence encoding the PCM-derived compound.

In some aspects, the bacterium strain comprises a disarmed Ti plasmid or disarmed Ri plasmid, a nucleotide sequence encoding a gene that induces PCM formation, and a nucleotide sequence encoding PCM-derived compound.

In some aspects, contacting the plant part with the bacterium strain comprises simultaneously introducing to the plant part: a first transgene associated with PCM formation, and a second transgene associated with the PCM-derived compound. And the method further comprising cultivating the plant part as transformed to generate PCM tissue, wherein the plant part is a seedling, a hypocotyl segment, a petiole, an internode, a node, a meristem, or a leaf.

In some aspects, contacting the plant part with the bacterium strain and culturing the plant part comprises contacting the plant part with a first bacterium strain comprising the nucleotide sequence encoding the gene that induces PCM formation, culturing the plant part to enhance PCM formation, contacting formed PCM tissue from the PCM with a second bacterium strain comprising the nucleotide sequence encoding the PCM-derived compound, and culturing the PCM tissue to enhance production of the PCM-derived compound by the PCM.

In some aspects, the bacterium strain comprises a Rhizobium rhizogenes strain selected from (such as being selected from the group consisting of) American Type Cell Culture (ATCC) 43057, ATCC 43056, ATCC 13333, ATCC 15834, and K599.

In some aspects, the method further comprises identifying the bacterium strain from a plurality of bacterium strains, wherein the bacterium strain is ATCC 43057, ATCC 43056, ATCC 13333, ATCC 15834, or K599.

In some aspects, the nucleotide sequence encoding the PCM-derived compound is operably connected to an inducible promoter, a strong promoter, or a root-tissue specific promoter.

In some aspects, the nucleotide sequence encoding the PCM-derived compound is operably connected to an ubiquitin promoter or a 35S Cauliflower Mosaic Virus promoter.

In some aspects, the method further comprises screening and selecting the cultured plant part for production of the PCM-derived compound or production of a metabolite of interest using end point reverse transcriptase PCR (RT-PCR) or fluorescent protein reporter expression in formed PCM tissue.

In some aspects, the method further comprises selecting PCM tissue from the cultured plant part for culturing in a culture medium, and screening the cultured PCM tissue for production of the PCM-derived compound or production of a metabolite of interest.

In some aspects, the method further comprises capturing the PCM-derived compound or the metabolite of interest by isolating and purifying the PCM-derived compound or the metabolite from the culture medium, from PCM tissue of the PCM, or combinations thereof.

In some aspects, the bacterium strain comprises a Rhizobium or Agrobacterium strain and the method further comprises transforming the Rhizobium or Agrobacterium strain to carry the nucleotide sequence encoding the PCM-derived compound using a vector containing: a right and left transferred DNA (T-DNA) border sequence; the nucleotide sequence encoding the PCM-derived compound, and a promoter.

Various aspects are directed to method of generating a bacterium strain comprising: transforming a bacterium strain with a nucleotide sequence encoding the PCM-derived compound, wherein the bacterium strain comprises a nucleotide sequence encoding a gene that induces PCM formation or is transformed to comprise the nucleotide sequence encoding the gene that induces PCM formation; and culturing the transformed bacterium strain.

In some aspects, transforming the bacterium strain comprises using a vector containing: a right and left T-DNA border sequence, the nucleotide sequence encoding the PCM-derived compound, a promoter.

In some aspects, the nucleotide sequence encoding the PCM-derived compound comprises an N-terminal tag.

Various aspects are directed to a PCM culture for producing a recombinant compound or a metabolite of interest, the PCM culture being induced from a plant part according to the method of claim 2 or 22, wherein a cell of the PCM culture comprises the nucleotide sequence encoding the PCM-derived compound.

Various aspects are directed to a PCM culture comprising a plurality of core precursor compounds each produced at a level that is greater than production in tissue of a wild-type plant.

In some aspects the plurality of core precursor compounds are compounds selected from (such as being selected from the group consisting of): an amino acid, a sugar, a phenol, an alkaloid, an isoprene, a terpene a sterol, fiber, a carbohydrate, a cannabinoid, a flavonoid, a fatty acid, and a combination thereof.

In some aspects, the plurality of core precursor compounds are compounds selected from (such as being selected from the group consisting of): cannabisativine, anhydrocannabisativine, friedelin, epifriedelanol, beta-amyrin, beta-sitosterol, campesterol, stigmasterol, cellulose, tetrahydrocannabinol (THC), cannabigerolic acid (CBGA), cannabidiolic acid (CBDA), carvone, dihydrocarvone, p-hydroxy-trans-cinnamamide, lignans, choline, orientin, vitexin, isovitexin, quercetin, luteolin, kaempferol, apigenin, and combinations thereof.

In some aspects, the PCM culture is generated from a Cannabaceae plant, a Brassicaceae plant, a Solanaceae plant, a Fabaceae plant, or an Apiacea plant.

Various aspects are directed to system comprising a plurality of bioreactors in serial connection, wherein each bioreactor is inoculated with the PCM obtained according to the method of claim 1 or 2, and configured for growth and maintenance of the PCM in a culture medium.

In some aspects, the culture medium comprises a liquid culture medium.

In some aspects, at least one bioreactor is a flask, plastic sleeve reactor, a bubble reactor, a mist reactor, an airlift reactor, a liquid-dispersed reactor or a bioreactor configured to generate micro- or nano-bubbles.

In some aspects, wherein each bioreactor of the plurality is structurally and operationally similar.

Various aspects are directed to a protein or metabolite of interest generated using the PCM obtained according to the method of claim 1 or 2.

Various aspects are directed to a method comprising transforming a plurality of plant parts with a plurality of bacterium strains to induce PCM formation, therefrom, assessing transformation frequencies of the plurality of bacterium strains, and selecting respective ones of the plurality of bacterium strains based on the transformation frequencies.

The respective ones of the plurality of bacterium strains is ATCC 43057, ATCC 43056, ATCC 13333, ATCC 15834, K599, or a combination thereof.

### DESCRIPTION OF THE DRAWINGS

The foregoing aspects and many of the advantages of this invention will become more readily understood by reference to the following detailed description, when taken in conjunction with the accompanying drawings, wherein:

FIG. 1 illustrates an example method for inducing PCM formation, consistent with the present disclosure.

FIG. 2 illustrates an example method for generating a PCM-derived compound using a PCM, consistent with the present disclosure.

FIG. 3 illustrates an example method for transforming a bacterium strain to comprise a sequence encoding a PCM-derived compound, consistent with the present disclosure.

FIG. 4 illustrates an example method for transforming a plant part to induce PCM formation and production of a PCM-derived compound, consistent with the present disclosure.

FIG. 5 illustrates an example expression construct for delivery of a sequence encoding a PCM-derived compound, consistent with the present disclosure.

FIGS. 6A-6K illustrate example binary vectors for delivery of a sequence encoding a recombinant compound, consistent with the present disclosure.

FIGS. 7A-7B illustrate example seedling preparations, consistent with the present disclosure.

FIGS. 8A-8G illustrate growth of transgenic PCM tissue, consistent with the present disclosure.

FIGS. 9A-9B illustrate cultured transgenic PCM tissue, consistent with the present disclosure.

FIG. 10 illustrates example data of transgenic PCM tissue that produces a recombinant compound, consistent with the present disclosure.

FIGS. 11A-11D illustrate example data showing production of a recombinant compound from PCM cultures, consistent with the present disclosure.

FIG. 12 illustrates an example expression cassette for the production of an albumin from a PCM culture, consistent with the present disclosure.

FIGS. 13A-13B illustrate example data showing production of ovalbumin from PCM cultures, consistent with the present disclosure.

FIGS. 14A-14B illustrate further example data showing production of ovalbumin from PCM cultures, consistent with the present disclosure.

FIGS. 15A-15B illustrate further example data showing production of ovalbumin from PCM cultures, consistent with the present disclosure.

FIGS. 16A-16C illustrate further example data showing production of ovalbumin from PCM cultures, consistent with the present disclosure.

FIGS. 17A-17D illustrate example images of PCM cultures producing betacyanin, consistent with the present disclosure.

FIGS. 18A-18B illustrate example images of PCM cultures producing betacyanin at different levels, consistent with the present disclosure.

FIGS. 19A-19F illustrate example images of PCM cultures producing betacyanin at different levels, consistent with the present disclosure.

FIGS. 20A-20C illustrate example images of PCM cultures producing betanidin and betaxanthin, consistent with the present disclosure.

FIGS. 21A-21B illustrate example images of PCM cultures producing betaxanthin, consistent with the present disclosure.

FIGS. 22A-22B illustrate example images of betalains in liquid from PCM cultures, consistent with the present disclosure.

FIGS. 23A-23B illustrate example experimental results from PCM cultures producing betalains, consistent with the present disclosure.

FIGS. 24A-24B illustrate example images of PCM cultures generated from hop plants, consistent with the present disclosure.

FIGS. 25A-25D illustrate example images of PCM cultures generated from hop plants, consistent with the present disclosure.

FIGS. 26A-26B illustrate example images of PCM cultures generated from hop plants, consistent with the present disclosure.

FIGS. 27A-27D illustrate example images of PCM cultures generated from Cannabis plants, consistent with the present disclosure.

FIGS. 28A-28B illustrate example images of PCM cultures generated from solanum tuberosum plants, consistent with the present disclosure.

FIGS. 29A-29B illustrate example images showing betalain production in solanum tuberosum PCM cultures, consistent with the present disclosure.

FIGS. 30A-30B illustrate example images of betalain production in solanum tuberosum PCM cultures using different bacterium strains, consistent with the present disclosure.

FIGS. 31A-31D illustrate example images of PCM cultures transformed to overexpress MYB transcription factors and produce anthocyanin, consistent with the present disclosure.

FIGS. 32A-32C illustrate example images of PCM cultures transformed to overexpress MYB transcription factors and produce anthocyanin, consistent with the present disclosure.

### DETAILED DESCRIPTION

The present disclosure is directed to methods, materials, and systems for transforming plant parts to induce PCM formation and/or the production of a PCM-derived compound by the PCM. Various aspects are directed to plant PCM tissue and/or cultures transformed to produce a PCM-derived compound and systems for production and recovery of the PCM-derived compound and/or produced metabolite using the transformed plant cells in the PCM tissue.

Plants can be used to produce tissue and different types of compounds for different applications. For example, plants can be cultured and grown to produce biomass, and in some instances, the biomass can express or produce particular compounds. In some embodiments, as plants have eukaryotic compound synthesis pathways, compounds can be produced or expressed by plants that are similar to proteins or other compounds expressed in mammals. Plant-based biomass production via outdoor agriculture, such as growing plants in the field and for the harvesting of compounds from the plant biomass, can be labor and time intensive, as well as requiring large areas of land to produce sufficient amounts of biomass. Further, particular plant-derived compounds can be difficult or unstainable to obtain from wild-type plants or plant tissue due to variability in production by the plants and/or low production level in plant tissue. In some instances, whole plant transformation systems present significant limitations to expressing different compounds in plant biomass product, requiring extensive time, labor and materials to develop and implement specialized protocols. For example, endogenous compounds can be produced in wild-type plants at levels which are costly and difficult to utilize for mass production. In some embodiments, proteins and other compounds of interest can be derived from animals using animal cell lines, which can be costly and difficult to mass express. For example, compounds derived from plants can be produced at levels that are insufficient for mass production.

Embodiments of the present disclosure are directed to transforming a plant part to induce PCM formation and, optionally, to induce production of a PCM-derived compound. In some embodiments, a bacterium strain can be used to transform the plant part. For example, Rhizobium strains, Agrobacterium strains, and other Rhizobia strains capable of inducing PCM phenotype in plants can be used to transform the plant part and to produce the PCM culture. The bacterium strain can be any strain harboring a root inducing (Ri) plasmid or otherwise being transformed to induce PCM formation, as further described herein. Embodiments are not limited to use of a bacterium strain to induce PCM formation, and can include contacting a plant part with a nucleotide sequence to transform the plant part and without a bacterium strain. For example, the plant part can otherwise be contacted with a (heterologous) nucleotide sequence encoding the Ri plasmid and/or gene that induces PCM formation, which transforms plant cells to express the nucleotide sequence. The resulting PCM can be used to produce PCM-derived compounds in a sustainable (environmentally and/or otherwise) and more-reliable manner than in wild-type or other plant tissue, and can provide a secure and reliable supply source of the PCM-derived compounds.

As used herein, a PCM includes and/or refers to plant cells transformed by a nucleotide sequence encoding a gene that induces PCM formation, which can include a plurality of different plant cell types and can be used to produce a PCM-derived compound. In some embodiments, the PCM is a tissue culture including the transformed plant cells, e.g., the plurality of different plant cell types. The PCM can include plant cell types including, but not limited to, plant stem cells, maturing cells, and mature cells. In some examples, the PCM is produced by infecting plant cells with the bacterium strain to induce hairy root formation. In some examples, the plant cells are infected with the bacterium strain, or otherwise contacting with the nucleotide encoding the gene that induces PCM formation, to induce PCM phenotype and to form the PCM. For example, the PCM is formed by isolating the tissue associated with the PCM phenotype from the wild-type tissue, such that the PCM culture may not include any wild-type tissue.

The PCM-derived compound, as used herein, includes and/or refers to a compound derived from the PCM, which can be an endogenous compound or a recombinant compound. For example, the PCM-derived compound can include an endogenous compound, such as a core precursor compound and other types of compounds. As used herein, a recombinant compound includes and/or refers to a compound not naturally produced by the plant and/or produced at levels below a threshold, and which is produced at an increased level by the PCM, as compared to a wild-type plant, due to transforming the plant cells with a nucleotide sequence encoding a recombinant compound. The nucleotide sequence may be heterologous to the plant. The core precursor compound includes and/or refers to a compound naturally produced by the plant, which is produced at an increased level by the PCM as compared to production in tissue of a wild-type plant and/or a plant grown in the field, via the induced generation of the PCM and/or enhanced by physical or chemical elicitor (e.g., production of the compound can be enhanced by light, temperature, pH, osmotic stress, hormones or other chemicals). In some embodiments, the recombinant compound can cause increased production of a metabolite of interest. As used herein, a metabolite of interest includes and/or refers to a metabolite compound produced by the PCM through a bio-pathway associated with a recombinant compound. The metabolite of interest may not be naturally produced by the plant and/or can be produced at levels below a threshold, and which is produced at an increased level by the PCM, as compared to a wild-type plant, due to transforming the plant cells with a (heterologous) nucleotide sequence encoding the recombinant compound or upregulating an endogenous compound.

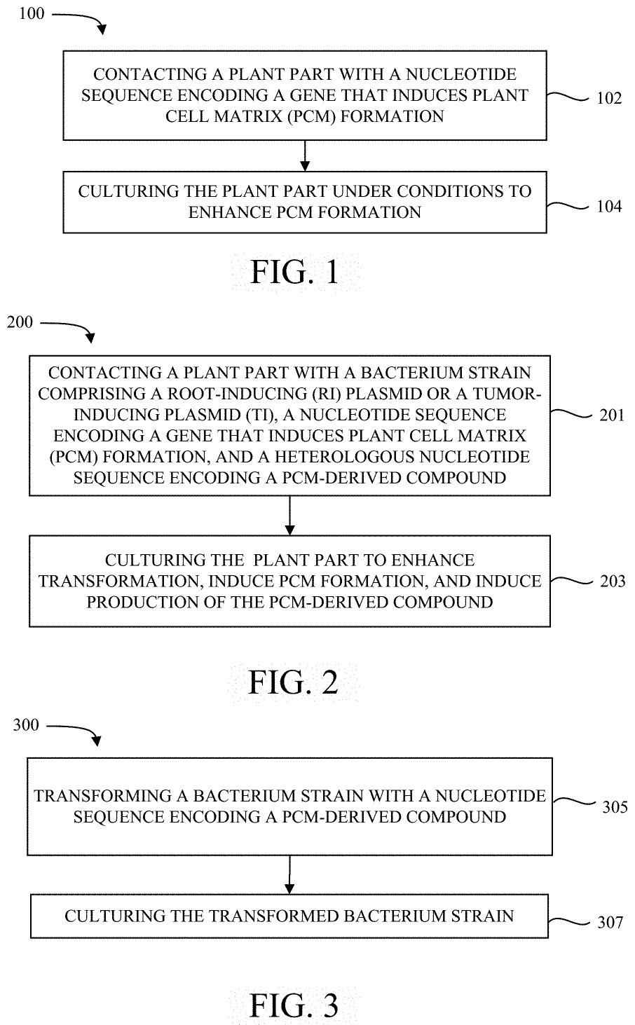

Turning now to the figures, FIG. 1 illustrates an example method for inducing PCM formation, consistent with the present disclosure.

At 102, the method 100 includes contacting a plant part with a nucleotide sequence encoding a gene that induces PCM formation (e.g., PCM gene). The plant part can be a seedling or a hypocotyl segment, although embodiments are not so limited and can include plant cells or other plant parts, such as a petiole, internode, node, meristem, or leaf

The nucleotide sequence can be heterologous to the plant. As described below, the contact with the nucleotide sequence can be performed using a variety of different techniques and which may transform cells of the plant part to express the nucleotide sequence and form a PCM. In some embodiments, as further described below, the nucleotide sequence can include or encode a Ri gene or plasmid that is expressed by plant cells of the plant part in response to the contact. In various embodiments, the gene that induces PCM formation encoded by the nucleotide sequence can include a plurality of genes that induce PCM formation (e.g., a plurality of PCM genes) and/or a plurality of nucleotide sequence can encode the plurality of PCM genes, such as a plurality of different PCM genes.

Such techniques and/or methods for contacting the plant part with the nucleotide sequence to transform the plant part and induce PCM formation include, but are not limited to, particle bombardment mediated transformation (e.g., Finer et al., 1999, Curr. Top. Microbiol. Immunol., 240:59), protoplast electroporation (e.g., Bates, 1999, Methods Mol. Biol., 111:359), viral infection (e.g., Porta and Lomonossoff, 1996, Mol. Biotechnol. 5:209), microinjection, liposome injection, polyethylene glycol (PEG) delivery to protoplasts, and agroinfiltration. Other example techniques can be used to facilitate uptake by a cell of the nucleic acid include calcium phosphate and other chemical mediators of intracellular transport, microinjection compositions, and homologous recombination compositions (e.g., for integrating a gene into a preselected location within the chromosome of the cell). Other example techniques can involve the use of liposomes, electroporation, or chemicals that increase free DNA uptake, transformation using viruses or pollen and the use of microprojection. Various molecular biology techniques are common in the art (e.g., Sambrook et al., 1989, Molecular Cloning: A Laboratory Manual, 2nd ed., Cold Spring Harbor Laboratory Press, New York). Transformation methods can include the use of liposomes, electroporation, chemicals that increase free DNA uptake, injection of the DNA directly into the plant, particle gun bombardment, transformation using viruses and microprojection.

In some embodiments, the contact of the plant part with the nucleotide sequence to transform the plant part and induce PCM formation can be provided via agroinfiltration. In some embodiments, the transformation is provided via Agrobacterium-mediated transformation (e.g., Komari et al., 1998, Curr. Opin. Plant Biol., 1:161), including floral dip transformation. Agroinfiltration can induce transient expression of gene(s) in a plant part to produce the PCM and/or PCM-derived compound, by injecting a suspension including the bacterium strain containing the gene or genes of interest into the plant part. In some embodiments, the transformation can be performed by an Agrobacterium-mediated gene transfer. The Agrobacterium-mediated gene transfer can include the use of plasmid vectors that contain DNA segment(s) which integrate into the plant genomic DNA. Methods of inoculation of the plant tissue vary depending upon the plant species and the Agrobacterium delivery system. The transformation can be performed with any suitable tissue explant that provides a source for initiation of whole-plant differentiation (See Horsch et al., 1988. Plant Molecular Biology Manual A5, 1-9, Kluwer Academic Publishers, Dordrecht).

In some embodiments, the agroinfiltration technique can be implemented as described in PCT application PCT/US21/25067, entitled “Agrobacterium-mediated Infiltration of Cannabis”, filed on Mar. 31, 2021, which is fully incorporated herein for its teaching and sometimes herein referred to as the “agroinfiltration protocol”.

In some embodiments, the transformation can be performed by a direct DNA uptake. There are various methods of direct DNA transfer into plant cells. In electroporation, the protoplasts are exposed to a strong electric field, opening up mini-pores to allow DNA to enter. In microinjection, the DNA is mechanically injected directly into the cells using micropipettes. In microparticle bombardment, the DNA is adsorbed on microprojectiles such as magnesium sulfate crystals or tungsten particles, and the microprojectiles are physically accelerated into cells or plant tissues. In some embodiments, the transformation or the re-transformation, as further described herein, is performed in tissue culture on media and in a controlled environment. “Media” refers to the numerous nutrient mixtures that are used to grow cells in vitro, that is, outside of the intact living organism.

As described above, in various embodiments, contacting the plant part with the nucleotide sequence, at 102 of method 100, includes contacting the plant part with a bacterium strain comprising the nucleotide sequence encoding the gene that induces PCM formation to transform the plant part and induce PCM formation. For example, the plant part can be contacted with the bacterium strain via submersion, spraying, dripping, and/or other forms of contact. In some embodiments, as further described herein, the contact can include contact with a liquid culture containing the bacterium strain, sometimes herein referred to as a “liquid bacterium medium”.

In some embodiments, the contact with the bacterium strain can be under infection conditions that induce and/or enhance transformation of the plant part to express the PCM phenotype. The infection conditions can include use of the liquid bacterium medium, a type of bacterium, and/or a type or an amount of contact with the bacterium stain, among other conditions. The bacteria strain can include the specific species or line of bacteria. The type or amount of contact with the bacterium strain can include immersion, spraying, dripping, and/or other contact in a time range of one to five days for co-cultivation.

The bacterium strain can include any strain capable of inducing PCM formation. As described above, the bacterium strain can include a Rhizobia strain, such as a Rhizobium strain or Agrobacterium strain. In some embodiments, the bacterium strain includes a Rhizobium rhizogenes strain (R. rhizogenes), formerly known as Agrobacterium rhizogenes. R. rhizogenes is a Rhizobium species that can be used to transform plant cells and is sometimes preferred due to high virulence and rapid development of transgenic materials in the form of hairy roots and/or a PCM. These Rhizobium strains have not been disarmed, meaning that the Rhizobium strains contain their original T-DNA which causes hairy root disease symptoms on infected plants contained on the Ri plasmid. PCM resulting from R. rhizogenes infection of plant tissue carry the T-DNA from the Ri plasmid and form vascular connections with their plant hosts. These vascular connections allow the PCM tissue to function similarly to wild-type roots, and can grow aggressively and out-compete wild-type roots. The T-DNA(s) from the bacterium strain can be stably integrated in the plant part. In some embodiments, the plant part can be transiently or stably transformed or modified in response to the contact with the bacterium strain, and which causes formation of the PCM.

In some embodiments, the PCM transgene is transferred to plant cells in response to the contact with the bacterium strain, which can cause an infection, and optionally along with any secondary transgene introduced into the bacterium strain, using electroporation and other cloning techniques. For example, in addition to the T-DNA from the Ri plasmid, additional T-DNAs can be co-delivered to the plant part and expressed in PCMs as transgenic PCM tissue, such as those from vectors carrying a gene associated with the recombinant compound. In other embodiments, the plant part can be infected with a first bacterium strain to produce the PCM and then the formed PCM can be transformed with a second bacterium strain to produce the PCM-derived compound, sometimes herein referred to as “re-transformation” or “re-transformed”. In some embodiments, PCMs are generated that are induced to produce a PCM-derived compound, as further described below, and may not be transformed with a secondary transgene. In other embodiments, the transformation and/or re-transformation can be performed without use of the bacterium strain, such as using any of the above-described techniques.

Contacting the plant part with the nucleotide sequence encoding the gene that induces PCM formation, such as contact with the bacterium strain, can transform the plant part to express the PCM phenotype, and optionally, to produce a PCM-derived compound, as further described below. The transformation of the plant part can be transient or non-transient, e.g., stable. A stable transformation includes or refers to the nucleotide sequence being integrated into the plant genome and as such represents a stable and inherited trait. A transient transformation includes or refers to a nucleotide sequence being expressed by the plant cell transformed but may not integrated into the genome, and as such represents a transient trait. As used herein the term “transformation” or “transforming” can include or refer to a process by which foreign DNA, such as an expression construct including the DNA, enters and changes wild-type DNA.

In some embodiments, the method 100 includes selecting the particular bacterium strain. Selection of an effective strain for the production of PCMs can depend on the plant species to be infected and can be determined empirically. Parameters such as the PCM tissue induction percentage per total explants, the PCM tissue initiation days per total explants, and the PCM tissue induction frequency per single explant can be measured to select the bacterium strain.

Many strains of R. rhizogenes exist and can be used for plant transformation. The strain can be an octopine, agropine, nopaline, mannopine, or cucumopine strain. Suitable strains of R. rhizogenes for use can include American Type Cell Culture (ATCC) 43057, ATCC 43056, ATCC 13333, ATCC 15834, and K599. In some embodiments, the bacterium strain is ATCC 43057, ATCC 43056, ATCC 13333, or ATCC 15834. In some embodiments, the bacterium strain is ATCC 43057, ATCC 43056, or ATCC 13333. In such embodiments, the bacterium strain used to infect the plant part can include an Ri plasmid that includes the nucleotide sequence encoding the gene that induces PCM formation. For example, the Ri plasmid carries the gene that induces PCMs, sometimes herein referred to as “the PCM gene” for ease of reference.

However, embodiments are not so limited. In some embodiments, the bacterium strain can be transformed to carry the PCM gene. For example, the bacterium strain can include a Ti plasmid and may not carry the gene that induces PCM formation. A Ti plasmid can carry a gene capable of inducing tumors. The Ti plasmid can be disarmed by deleting the tumor inducing gene and introducing the gene that induces PCM formation using a T-DNA. In some examples, the bacterium strain can be transformed to include a disarmed Ti plasmid and the nucleotide sequence encoding the gene that induces PCM formation. In some embodiments, no bacterium strain is used to transform the plant cells and induce PCM formation. For example, the vector(s) carrying the gene(s) can be used to transform the plant cells of the plant part without the use of a bacterium strain.

The method used for bacterium strain infection can vary, but can include the preparation of a fresh wild-type shoot (cut at the stem) or seedling (cut at the hypocotyl) cuttings, and inoculation of the cut end with the bacterium strain. Cocultivation of the plant part on media can facilitate delivery of both a Ri plasmid and vector T-DNA(s) to the wild-type tissue. Binary, superbinary, pGreen or co-integrate vectors containing appropriate genes (e.g., encoding the recombinant compound) and selectable markers and/or reporter genes can be prepared and transferred into the bacterium strain. Suitable vectors contain right and left T-DNA border sequences to allow for delivery of the DNA into the plant cells. In various embodiments, as further illustrated by FIG. 3, the method 100 can include transforming a wild-type bacterium strain with the nucleotide sequence encoding a recombinant compound, and, in some embodiments, with the gene that induces PCM formation.

The method 100 can also include preparing a plant part, such as an explant, to be inoculated with the prepared bacterium strain or otherwise contacted with the nucleotide sequence encoding the gene that induces PCM formation. Cells of the plant part can be transformed with an expression construct suitable for production of the PCM, and optionally, a recombinant compound. Different plant parts, such as hypocotyl, leaf, stem, stalk, petiole, shoot tip, cotyledon, protoplast, storage root, meristem, node, internode, or tuber, can be used to induce PCM formation. For different species, the most efficient explant material can vary in tissue/organ source and age. Juvenile material (e.g., from one to five days germinated seed, three to ten day seedling) can be optimal for at least some plants. The explant can include plant tissue that has been wounded. The wounded tissue can be infected by contact with or immersion into a prepared bacterium strain culture or otherwise contacted with the nucleotide sequence. For example, the plant tissue can be immersion into and/or submerged in the bacterium strain culture. Appropriate media and incubation conditions for infection or contact, co-cultivation, and/or PCM growth induction can depend on the explant to be transformed. The infected explant can be cultured to enhance or optimize transformation and PCM induction and development, as further described herein.

At 104, the method 100 further includes culturing the plant part under growth conditions to enhance PCM formation. In various embodiments, the plant part can be cultured with the bacterium strain or otherwise is contacted with the nucleotide sequence encoding the PCM gene (which is heterologous to the plant) to induce PCM formation, and then cultured in another culture medium or a plurality of culture mediums to enhance further PCM tissue growth. More particularly, and in some embodiments, the plant part is contacted and co-cultured with the bacterium strain under the infection conditions to transform the plant part and for a period of time (e.g., one to five days). After the period of time, the bacterium strain is removed and/or killed, such as using antibiotics, and the transformed plant part is cultured under the growth conditions using a culture medium.

The growth conditions can include a liquid culture medium, a type of culture medium, a type or amount of contact with the culture medium, and a plant type. The liquid culture medium can include a culture medium in a liquid form. The type of culture medium can include a liquid-based medium containing sugar and Driver and Kuniyuki Walnut (DKW) basal salts, Murashige and Skoog (MS) basal salts, or Woody Plant basal salt mixtures (WPM), herein sometimes generally referred to as “DKW”, “MS”, and “WPM” for ease of reference. In some embodiments, the type of culture medium can include a culture medium containing a pH buffer, such as 2-(N morpholino) ethanesulfonic acid (MES) buffer (e.g., 1 g/L of MES buffer), among other types of buffers, such as bis-tris buffer. The pH buffer can prevent or mitigate pH shifts. In some embodiments, the culture medium can include a liquid-based medium containing sugar, DKW or MS, and a pH buffer, among other components. However, embodiments are not limited to liquid culture mediums and can include solid culture mediums with sugar, DKW, MS, and/or a pH buffer. The type or amount of contact with the culture medium can include an intermittent contact, spraying, dripping, and/or contact or contact cycle in a time range of one week to three months. As further described below, in some embodiments, the growth conditions can additionally include providing supplemental gas, such as oxygen, to the plant tissue.

The plant type can include a dicotyledonous plant. In some embodiments, the plant type can include a Cannabaceae plant, a Brassicaceae plant, a Solanaceae plant, a Fabaceae plant, or an Apiacea plant. In some embodiments, the plant type can include a Cannabaceae plant, a Brassicaceae plant, or a Solanaceae plant. In some embodiments, the plant type include a plant part selected from a seedling (e.g., hypocotyl), a petiole, an internode, a node, a meristem, and a leaf.

In some embodiments, the plant type can include a specific plant line and/or clone of the plant that exhibits greater PCM formation than other plant lines and/or clones. For example, within a plant species, there can be genetic variability which causes different optimized tissue formation from the PCM compared to other plant lines and/or clones. A plurality of plant lines and/or clones can be transformed to form PCMs and screened to identify the particular plant line and/or clone with the optimized PCM formation among the plurality of plant lines and/or clones after the contact with the nucleotide sequence encoding the PCM gene followed by culturing with a culture medium, such as liquid culture medium. In some embodiments, the specific plant line and/or clone of the plant can be screened for and/or selected by culturing the plurality of plant clones of different plant lines (and/or a plurality of plant clones of a plant line), as transformed by the nucleotide sequence, using an intermittent contact with the liquid culture medium or other type of culture medium containing the sugar and basal salt, as described above and further described below, and which can result in enhanced growth rates among the plurality of PCMs formed and with a greater dynamic range of growth rates among the plurality of PCMs as compared to PCMs formed using a constant contact with the liquid culture medium and/or use of other types of culture mediums (e.g., solid mediums) for inducing tissue growth of the plant part transformed to express the PCM phenotype. A dynamic range of growth rates can include a difference between the fastest growing PCM and the slowest growing PCM among the plurality of PCMs formed. By having a greater dynamic range, selection of the optimal or subset of optimal PCMs among the plurality can occur faster and/or more easily as compared to a lower dynamic range. An optimized or optimal PCM includes and/or refers to a PCM or subset of PCMs exhibiting the greatest growth rate(s) among the plurality of PCMs. For example, a user can visually select the optimized or subset of optimized PCMs among the plurality of PCMs. In some embodiments, the growth rates of the plurality of PCMs can be measured and compared to select the optimized PCM or subset of optimized PCMs.

In some embodiments, as noted above, the type of contact can comprise intermittently contacting the plant part with the culture medium, such as with a liquid culture medium. Intermittent contact, as used herein, includes and/or refers to cycling between contact of the plant part with the culture medium and no contact of the plant part with the culture medium. For example, the plant part transformed via contact with the nucleotide sequence encoding the gene that induces the PCM formation can be intermittently contacted with the culture medium to enhance PCM formation. By providing an intermittent contact, the transformed plant part is provided with nutrients (e.g., sugars and basal salts) for growth during times of contact with the culture medium, and is provided with air or other gases for growth during times of no contact with the culture medium. In contrast, with constant contact, parts of the PCM tissue of the PCM formed may be in the liquid or other types of media at all times and may not have access to air or other gases as needed for survival and/or growth. In some embodiments, the growth conditions can further include exposure to a supplemental gas and a type of gas. The supplemental gas can be provided to the plant part, such as during no contact times (e.g., no contact with the liquid culture medium). The liquid culture medium or other type of media can be drained or otherwise removed during the no contact times.

In some embodiments, the intermittent contact comprises cycling between contacting (e.g., submerging, dripping, or other types of contact) the plant part with the culture medium and not contacting (e.g., not submerging, dripping, or other types of contact) the plant part with the culture medium at a duty cycle of between 1 percent and 25 percent, such as with a liquid culture medium. A duty cycle, as used herein, refers to the percentage of time that the plant part is in contact with the culture medium as compared to the time the plant part is not in contact. For example, the plant part can be contacted for ten minutes and not contacted by the culture medium, such as a liquid culture medium, for fifty minutes, every hour over a total period of time of about one week (e.g., seven days) to about three months (e.g., ninety days) or more, resulting in a duty cycle of 16.67 percent over the total period of time. In some embodiments, the total period of time includes between about two weeks (e.g., fourteen days) and about three months, about two weeks and about two months (e.g., sixty days), about two weeks and about one month (e.g., thirty days), about twenty days and about three months, about twenty days and about two months, about twenty days and about one month, about one month and about three month, or about one month and about two months, among other ranges of periods of time.

In some embodiments, contacting the plant part with the nucleotide sequence encoding the PCM gene and culturing of the plant part under the growth conditions to enhance PCM formation can result in production of PCM tissue at an greater level than production of tissue (e.g., root tissue) by a wild-type plant or plant grown in the field. For example, the production of the PCM tissue by the PCM can be at least about two-fold to about 500-fold compared to production of tissue (e.g., root tissue) by the wild-type plant or plant grown in the field and/or at a growth rate of at least about 2-fold to about 500-fold compared to the production of the tissue by wild-type plant or plant grown in the field. In some embodiments, the production of the PCM tissue by the PCM can be at a growth rate that is about 2-fold to about a 500-fold, about a 4-fold to about a 500-fold, about an 8-fold to about a 500-fold, about a 10-fold to about a 500-fold, about a 15-fold to about a 500-fold, about a 20-fold to about a 500-fold, about a 20-fold to about a 400-fold, about a 20-fold to about a 300-fold, about a 20-fold to about a 100-fold, about a 15-fold to about a 400-fold, about a 15-fold to about a 300-fold, about a 15-fold to about a 200-fold, about a 15-fold to about a 100-fold, about a 15-fold to about a 50-fold, or about a 15-fold to about a 30-fold compared to the production of the tissue by wild-type plant or plant grown in the field. As used herein, growth rate includes and/or refers to an amount of root biomass produced in a period of time, which can include a mass level (e.g., grams (g)) of PCM tissue produced by the PCM in a period of time and can optionally be per unit of area. Mass level or mass includes and/or refers to the amount of biomass produced (e.g., grams per square meter per month of dry PCM tissue) by the PCM, such as grams of PCM tissue or root tissue. For example, the PCM can produce PCM tissue at a greater mass level than root tissue produced by a wild-type-plant or as grown in the field.

In some embodiments, the PCM can produce PCM tissue at a mass level that is at least a 2-fold (or times), at least a 3-fold, at least a 4-fold, at least an 8-fold, at least a 10-fold, at least a 15-fold, at least an 18-fold, at least a 20-fold, at least a 25-fold at least a 30-fold, at least a 40-fold, at least a 50-fold, at least a 100-fold, at least a 200-fold, or at least a 500 fold increase as compared to the root tissue produced by a wild-type plant and/or as grown in the field. In some embodiments, PCM can include PCM tissue at a growth rate or mass level that is at about a 2-fold to about a 500-fold, about a 4-fold to about a 500-fold, about an 8-fold to about a 500-fold, about a 10-fold to about a 500-fold, about a 15-fold to about a 500-fold, about a 20-fold to about a 500-fold, about a 20-fold to about a 400-fold, about a 20-fold to about a 300-fold, about a 20-fold to about a 100-fold, about a 15-fold to about a 400-fold, about a 15-fold to about a 300-fold, about a 15-fold to about a 200-fold, about a 15-fold to about a 100-fold, about a 15-fold to about a 50-fold, or about a 15-fold to about a 30-fold, among other range increases in the PCM tissue mass as compared tissue mass (e.g., root tissue) produced by a wild-type plant and/or as grown in the field.

In some embodiments, the method 100 includes culturing the plant part under the growth conditions to induce and enhance PCM formation and to induce production of a PCM-derived compound by the PCM formed. In various embodiments, the plant part can be cultured with the bacterium strain to induce PCM formation or otherwise contacted with the nucleotide sequence encoding the PCM gene, and then cultured in another culture medium or a plurality of culture mediums to induce production of the PCM-derived compound, such as in liquid or solid culture mediums. In some embodiments, the plant part is transformed using the first transgene that induces the PCM phenotype to produce PCM tissue and the PCM tissue is isolated from wild-type tissue and produces PCM-derived compound(s), as further described herein. In some embodiments, the plant part is further transformed using a second transgene that induces the production of the PCM-derived compound, as further described herein.

The PCM-derived compound can include a recombinant compound and/or a core precursor compound, as previously described. In some examples, the PCM-derived compound can include an endogenous compound that is produced by the PCM at greater levels than a wild-type plant. In some embodiments, the PCM-derived compound can include a heterologous compound. The PCM-derived compound can be expressed at greater levels than a wild-type plant, which can be due to the transformation and/or culturing conditions. For example, an endogenous compound can be produced by the PCM-derived compound at a greater level than the wild-type plant and/or a field grown plant.

In some embodiments, the PCM can express or produce the PCM-derived compound by at least a 2-fold (or times), at least a 3-fold, at least a 4-fold, at least an 8-fold, at least a 10-fold, at least a 15-fold, at least an 18-fold, at least a 20-fold, at least a 25-fold at least a 30-fold, at least a 40-fold, at least a 50-fold, at least 100-fold, at least 200-fold, at least 500-fold increase in the level of production (e.g., mass level and/or growth rate) as compared to the production level of an endogenous compound in tissue of a wild-type plant and/or as grown in the field. In some embodiments, PCM can express or produce the PCM-derived compound between about a 2-fold to about a 500-fold, about a 4-fold to about a 500-fold, about an 8-fold to about a 500-fold, about a 10-fold to about a 500-fold, about a 15-fold to about a 500-fold, about a 20-fold to about a 500-fold, about a 20-fold to about a 400-fold, about a 20-fold to about a 300-fold, about a 20-fold to about a 100-fold, about a 15-fold to about a 400-fold, about a 15-fold to about a 300-fold, about a 15-fold to about a 200-fold, about a 15-fold to about a 100-fold, about a 15-fold to about a 50-fold, or about a 15-fold to about a 30-fold, among other range increases in the level production (e.g., mass level and/or growth rate) as compared to the production level of the endogenous compound in tissue of a wild-type plant and/or as grown in the field. As used herein, a production level and/or level of production includes and/or refers to a mass level and/or growth rate.

In various embodiments, contacting the plant part with the nucleotide sequence encoding the gene that induces PCM formation includes introducing a first transgene associated with PCM formation to the plant part and to transform the plant part, and cultivating the transformed plant part to generate PCM tissue. In some embodiments, as further described herein, the method 100 includes simultaneously introducing a first transgene and a second transgene to the plant part, and cultivating the plant part as transformed to generate PCM tissue, and cultivating the plant part as transformed to generate PCM tissue. The first transgene can be associated with PCM formation, and the second transgene can be associated with the PCM-derived compound. In some embodiments, the first transgene is naturally occurring in the bacterium strain and the second transgene is non-naturally occurring and/or transgenic. In some embodiments, both the first transgene and the second transgene are non-naturally occurring and/or transgenic.

In some embodiments, the first transgene and second transgene may be separately delivery to the plant part. For example, contacting the plant part with the nucleotide sequence and culturing the plant part comprises contacting the plant part the (first) nucleotide sequence encoding the gene that induces PCM formation, and culturing the plant part to enhance PCM formation, such as under the above-described growth conditions. Followed by contacting the formed PCM tissue a (second) nucleotide sequence encoding the PCM-derived compound, culturing the PCM tissue to enhance production of the PCM-derived by the PCM, such as under the above-described growth conditions.

The core precursor compound can include endogenous compounds which can be involved in different bio-pathways to form other compounds, such as a protein of interest or metabolite of interest. In some embodiments, a core precursor can include a metabolite. Example core precursor compounds include amino acids, organic acids, fatty acids, sugars, carbohydrates, phenolics, alkaloids, isoprenes, terpenes, sterols, fiber, and cannabinoids. Other example core precursor compounds include neutral detergent fiber (NDF), acid detergent fiber (ADF), crude fiber, carbohydrate, and crude fat. For example, pentenyl diphosphate (IPP) and dimethylallyl diphosphate (DMAPP) are core precursors to a variety of terpenes. Within the terpene bio-pathway, IPP and DMAPP core precursors are converted into other compounds such as geranyl diphosphate (GPP) and farnesyl diphosphate (FPP), which are in turn core precursors compounds to a variety of specialized monoterpenes (e.g. myrcene, limonene, linalool, carvone) and sesquiterpenes (e.g. bisabolol, farnesene, nerolidol) respectively. As another example, beta-amyrin is a precursor to producing a variety of different triterpenoids and saponins via different bio-pathways. As a further example, the amino acid tyrosine is a core precursor compound involved in diverse bio-pathways for potential metabolites of interest including betalains, lignin, isoquinolin alkaloids (e.g. morphine, cholchicine), and rosmarinic acid. Similarly, the amino acid phenylalanine is a core precursor compound in bio-pathways that produce metabolites of interest including rosmarinic acid and anthocyanins.More specific example core precursor compounds include cannabisativine, anhydrocannabisativine, friedelin, epifriedelanol, beta-amyrin, beta-sitosterol, campesterol, stigmasterol, cellulose, tetrahydrocannabinol (THC), cannabigerolic acid (CBGA), cannabidiolic acid (CBDA), carvone, dihydrocarvone, p-hydroxy-trans-cinnamamide, lignans, choline, orientin, vitexin, isovitexin, quercetin, luteolin, kaempferol, and apigenin, among others. In some embodiments, the core-precursor may be used to produce a metabolite of interest (e.g., monoterpenes, sesquiterpene, triterpenoids, saponins, lignin, isoquinolin alkaloids, rosmarinic acid) using a single conversion or step or a plurality of conversions or steps.

In some embodiments, the PCM can produce a combination of core precursor compounds. For example, the PCM can produce at least two or more amino acids, organic acids, fatty acids, sugars, carbohydrates, phenolics, phenylpropanoids, flavonoids, alkaloids, isoprenes, terpenes, sterols, fiber, and cannabinoids. In some embodiments, the PCM can produce alkaloids, triterpenes, and sterols at an increased level of production than in tissue (e.g., root tissue) of a wild-type plant. In some embodiments, the PCM can produce cannabisativine, anhydrocannabisativine, friedelin, epifriedelanol, beta-amyrin, beta-sitosterol, campesterol, and stigmasterol, each at an increased level of production as compared to the production level of the endogenous compound in tissue of a wild-type plant and/or as grown in the field.

In some embodiments, the PCM with the core precursor and/or combination of core precursors can include an elicitor. For example, the elicitor can be added to the PCM. An elicitor includes or refers to a molecule that physically or chemically triggers a response in plant cells of the PCM and induce production of certain classes of compounds. For example, the production of the PCM-derived compound can be enhanced by the elicitor and in response to stimuli, such as light, temperature, pH, and/or osmotic stress, among other stimuli.

The recombinant compound can include a protein of interest or a production source for a metabolite of interest. Example proteins of interest include allergens, vaccines, enzymes, enzyme inhibitors, antibodies, antibody fragments, antigens, toxins, anti-microbial peptides, hormones, growth factors, blood proteins, receptors, signaling proteins, fusion or labelled proteins, albumin, coagulation factors, immunoglobulins, transferrin, sulphatases, digestive enzymes, lipases, pepsin, trypsin, monoclonal antibodies, interleukins, sugars, and interferons. Example production sources for metabolites include enzymes, transcription factors, and regulatory proteins. In some embodiments, the recombinant compound can cause overexpression of a metabolite of interest. Example metabolites include acetylenes, thiophenes, glycosides, glucosinolates, purines, pyrimidines, alkaloids, phenols, phenolics (e.g., quinones), phenylpropanoids, betalains, glycosides, terpenoids (e.g., iridoids, sesquiterpenes, diterpenoids, and triterpenoids), polyketide, lignans, a fatty acid synthase product or a phloroglucinol, cannabinoids, and flavonoids, among others.

In embodiments involving a PCM-derived compound and a bacterium strain, the bacterium strain can be prepared for infecting the plant part by introducing a nucleotide sequence encoding the PCM-derived compound, such as a recombinant compound, into the bacterium strain (e.g., by electroporation) and culturing the transformed bacterium strain under conditions to select positively transformed cells.

In some embodiments, and as noted above, culturing a transformed plant part can include inducing formation of PCM tissue from the transformed plant part and culturing the PCM tissue in a culture medium under the growth conditions for production of the PCM-derived compound. For example, the method 100 can include screening new growth from the cultured transformed plant part for PCM formation. For PCM induction, the plant part can be transferred into solid culture media with antibiotics two or three days after infection or co-cultivation, and for up to two or three months. Suitable antibiotics include cefotaxime sodium, carbencilin disodium, vancomycin, ampicillin sodium, claforan, streptomycin sulphate, and tetracycline, and combinations thereof. The amount of antibiotic to kill or eliminate redundant bacteria can range in concentration from 100 to 500 μg/mL. The PCM can be induced within a short period of time, which can vary from one week to three months, or more, depending on the plant species. In some embodiments, the PCM can be induced for two weeks to eight weeks via the contact with the culture medium (e.g., intermittent contact with a liquid culture medium). After the PCM is established (e.g., after culturing under the growth conditions), the PCM can be maintained in culture, and in some embodiments, so long as the PCM is transferred to fresh media every one to three weeks. In some embodiments, the decontaminated PCM tissue can be sub-cultured on hormone-free medium regularly (e.g., every one to two weeks).

The various described culture mediums (e.g., used to infect or an liquid infection medium, co-cultivate medium, selection medium, solid media) can each generally comprise water, a basal salt mixture, a sugar, and optionally other components such as vitamins, selection agents, amino acids, and phytohormones. At least some of the medias (e.g., for enhancing growth) can include sugars, basal salts, growth hormones, selection agents, and/or antibiotic agents, among other reagents, such as water and vitamins. For example, the various medias can include nutritional sources of nitrogen, phosphorus, potassium, sulfur, calcium, magnesium, iron, boron, molybdenum, manganese, cobalt, zinc, copper, chlorine, and iodine. Macroelements can be provided as NH₄NO₃, (NH₄)₂SO₄, KNO₃, CaCl₂.2H₂O, MgSO₄.7H₂O, and KH₂PO₄. Micro elements can be provided as KI, H₃BO₃, MnSO₄.4H₂O, ZnSO₄, Na₂MoO₄ 2H₂O, CuSO₄.5H₂O, CoCl₂.6H₂O, CoSO₄.7H₂O, FeSO₄.7H₂O, and Na₂EDTA.2H₂O. Organic supplements such as nicotinic acid, Pyridoxine-HCl, Thiamine-HCl, and glycine can be included. Generally, the pH of the medium is adjusted to 5.7±0.5 using dilute KoH and/or HCl. Solid plant culture media can further include a gelling agent such as, for example, gelrite, agar or agarose.

In some embodiments, a respective culture medium, such as the above-noted solid culture medium, can include selection agents, phytohormones and/or plant growth regulators such as, for example, auxins, cytokinins, or gibberellins. The phytohormones can be selected from free and conjugated forms of naturally occurring phytohormones or plant growth regulators, or their synthetic analogues and precursors. Naturally occurring and synthetic analogues of auxins include, but are not limited to, indoleacetic acid (IAA), 3-indolebutyric acid (IBA), a-napthaleneacetic acid (NAA), 2,4-dichlorophenoxyacetic acid (2,4-D), 4-(2,4-dichlorophenoxy)butyric acid, 2,4,5-trichlorophenoxyacetic acid (2,4,5-T), 3-amino-2,5-dichlorobenzoic acid (chloramben), (4-chloro-2-methylphenoxy)acetic acid (MCPA), 4-(4-chloro-2-methylphenoxy)butanoic acid (MCPB), mecoprop, dicloprop, quinclorac, picloram, triclopyr, clopyralid, fluoroxypyr, dicamba and combinations thereof. Any combination of two or more auxins can be present in the nutritive media. Natural cytokinins and synthetic analogues of cytokinins include, but are not limited to, kinetin, zeatin, zeatin riboside, zeatin riboside phosphate, dihydrozeatin, isopentyl adenine 6-benzyladenine and combinations thereof. Any combinations of two or more cytokinins can be present in the mediums.

Presence of an effective amount of the auxin, and optionally an effective amount of the cytokinin, can promote cell division, improve regenerability, and/or induce the growth of more regenerative tissue. The effect of exogenous auxin to produce a morphological response can be enhanced by the addition of antioxidants, amino acids, cobalt, or AgNO₃. Casamino acids provide a source of organic nitrogen in the form of amino acids hydrolyzed from Casein that can tolerate high salt conditions without degrading. Glutamine, asparagine, and methionine play complex roles in regulation of biosynthetic pathways that result in morphogenic response.

In some embodiments, the new growth is screened to identify the transformed PCM tissue and the identified transformed PCM tissue is separated and sub-cultured in the culture medium (e.g., a selection and/or solid culture medium) under the growth conditions for production of the PCM-derived compound, such as production (e.g., overexpression) of a core precursor compound at a greater level than produced in tissue of a wild-type plant and/or a wild-type plant in the field. PCM tissue, as used herein, includes and/or tissue (e.g., roots) exhibiting the PCM phenotype. As used herein, the PCM phenotype includes and/or refers to roots that tend to resemble thick, fluffy cords as compared to wild-type roots that are long, thin, and smooth. The culture medium can include a liquid culture medium or a solid culture (e.g., growth) medium which is hormone-free, e.g., has an absence of added plant growth hormones. In various embodiments, the PCM tissue is isolated from photosynthetic wild-type tissue and, therefore, may not contain any remaining photosynthetic wild-type tissue. The resulting PCM culture similarly may not contain any remaining photosynthetic wild-type tissue. The absence of the added plant growth hormones can be used to select PCM tissue over wild type as the wild-type tissue can die in the absence of the growth hormone when grown in the dark. A second selection technique can be used, such as selection agent or reporter gene. In some embodiments, the culture medium can further include a selection agent, such as an antibiotic or herbicide to select tissue that produce the PCM-derived compound. For example, the cultured tissue can be screened for the production of the PCM-derived compound. In some embodiments, a reporter gene, such as yellow fluorescent protein (YFP) or red fluorescent protein (RFP), can be used to further transform the plant part and to allow for selection of the PCM tissue, such as PCM tissue that contains the second transgene.

In some embodiments, the PCM strains can be isolated and characterized. For example, the method 100 can include screening and selecting cultured (infected) plant parts for a PCM-derived compound (e.g., a recombinant compound) using end point RT-PCR or fluorescent protein reporter expression (e.g., RFP or YFP) in formed PCM tissue. However, examples are not so limited and other molecular biology methods can be used, such as DNA-sequencing, southern blot analysis, northern blot analysis, and/or western blot analysis.

In some embodiments, the nucleotide sequence associated with the PCM phenotype and/or the nucleotide sequence associated with the PCM-derived compound (e.g., a heterologous nucleotide sequence) is linked to a reporter gene marker for rapid detection of transformed PCM tissue. For example, the reporter gene can be linked to the Ri plasmid. In some embodiments, a DNA sequence encoding a string of six to nine histidine residues is present in an expression cassette for production of PCM-derived compound (e.g., the recombinant compound), as further described herein. The result is expression and/or production of a PCM-derived compound with a 6× His or poly-His-tag fused to its N- or C-terminus. The expressed His-tagged compounds can be detected because the string of histidine residues binds to several types of immobilized metal ions, including nickel, cobalt and copper, under specific buffer conditions. In addition, anti-His-tag antibodies are commercially available for use in assay methods involving His-tagged PCM-derived compounds.

Due to the site uncertainty of integration of the PCM gene and/or other nucleotide sequence(s) into the plant cell genome, PCM strains can show different production patterns for the PCM-derived compound. Production levels can be measured using biochemical analysis to quantify compound concentration in the medium (e.g., Lowry, Bradford, BCA, and UV spectroscopic protein assays). PCM strains having the desired pattern and level of production can be identified by the presence of the PCM-derived compound in the media. Subculture and selection can be performed repeatedly to obtain compound-producing PCM lines that secrete the protein of interest or the production source for the metabolite of interest at high levels on a biomass basis (e.g., per gram dry weight) and/or secrete a core precursor compound at higher levels than a wild-type plant.

To initiate a PCM culture in liquid medium, a piece of a transformed tissue (e.g., 1 gm piece) can be transferred to a culture vessel. Any conventional plant or culture medium can be used in the practice of the present invention; multiple plant culture media are commercially available as dry (powdered) media and dry basal salts mixtures, for example.

In some embodiments, the method 100 can include capturing the PCM-derived compound, such as a core precursor compound, recombinant compound and/or metabolite of interest. For example, the core precursor compound, recombinant compound and/or metabolite can be captured by isolating and purifying the recombinant compound and/or metabolite of interest from the culture medium including the PCM tissue and/or from PCM tissue. Recovery of a secreted core precursor compound, recombinant compound and/or produced metabolite of interest from the spent media can include primary recovery steps (e.g., conditioning and pretreatment) and purification steps (e.g., capture and polishing). The spent media is typically concentrated, clarified, and conditioned prior to a chromatography (capture) step. Conditioning and pretreatment of secreted core precursor compound, recombinant compound and/or metabolite of interest can include steps to maximize product binding by capture chromatography and the lifetime of capture chromatography media (e.g., affinity resins), reduce binding of plant components to the PCM-derived compound, and stabilize the protein and/or metabolite of interest for purification, such as conditioning by crossflow filtration, pH adjustment, and dead-end filtration, in any order. Typically, conditioning can include adjusting media pH, ionic strength, and buffer composition. Conditioning can further include removing plant impurities that can interfere with the method of purification, reducing overall plant protein burden; and reducing PCM-derived compound exposure to phenolics and proteases, such as by two-phase partitioning, adsorption, precipitation, and membrane filtration. Conditioning can further include a reducing the media volume (e.g., by cross-flow filtration).

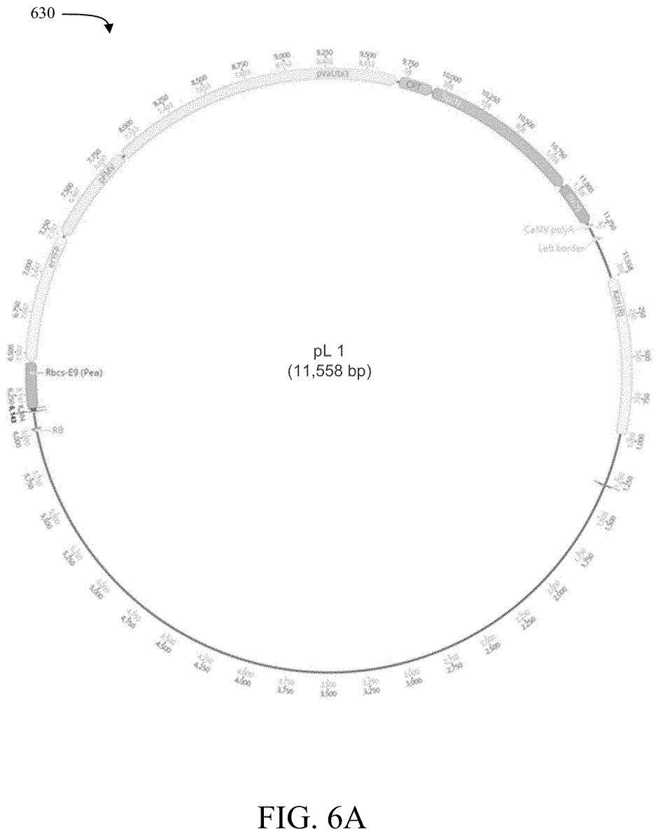

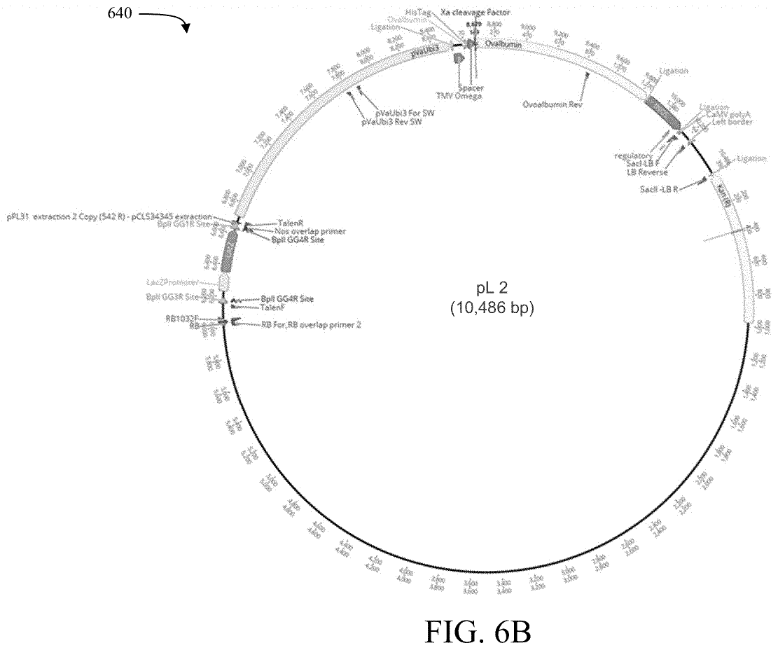

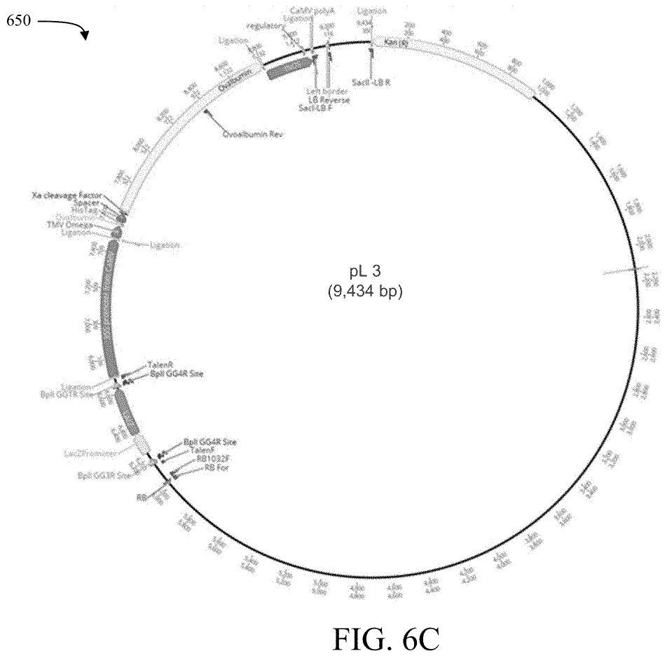

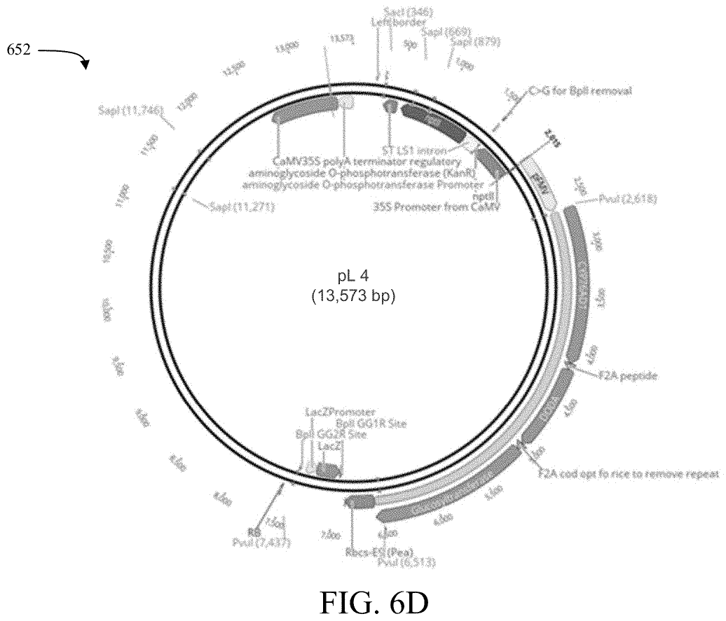

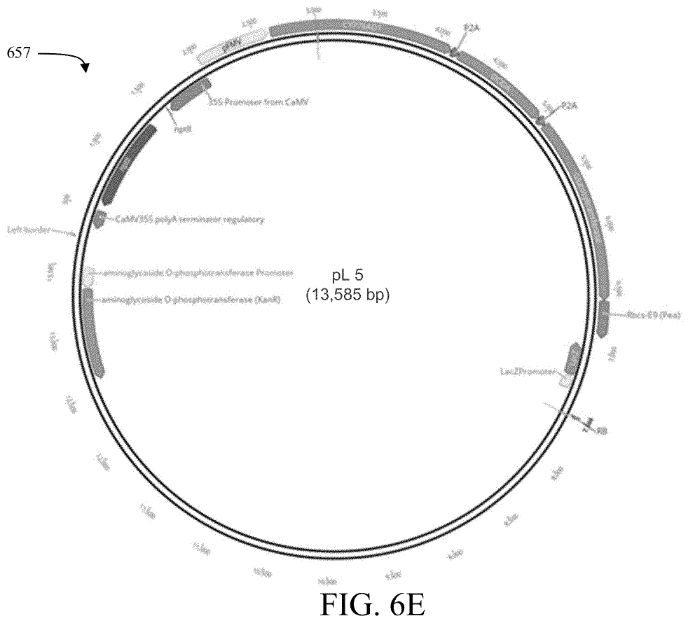

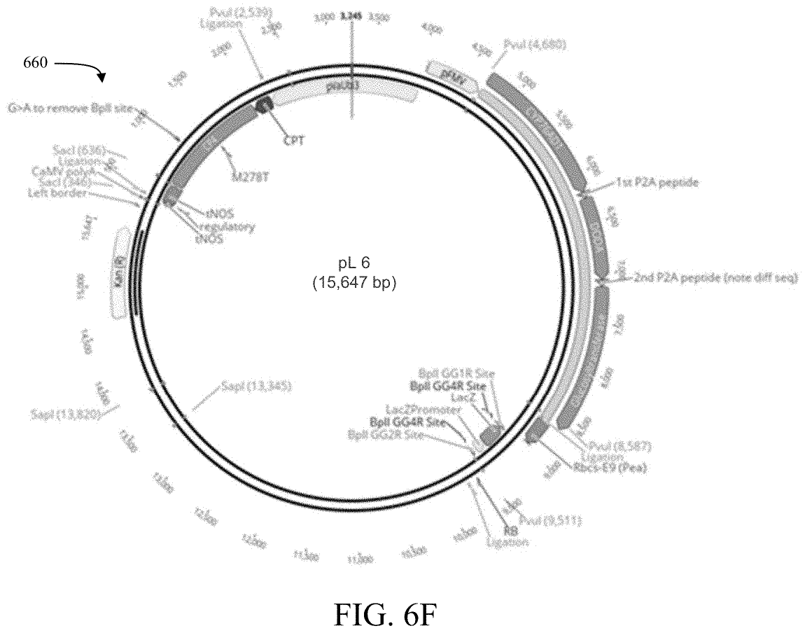

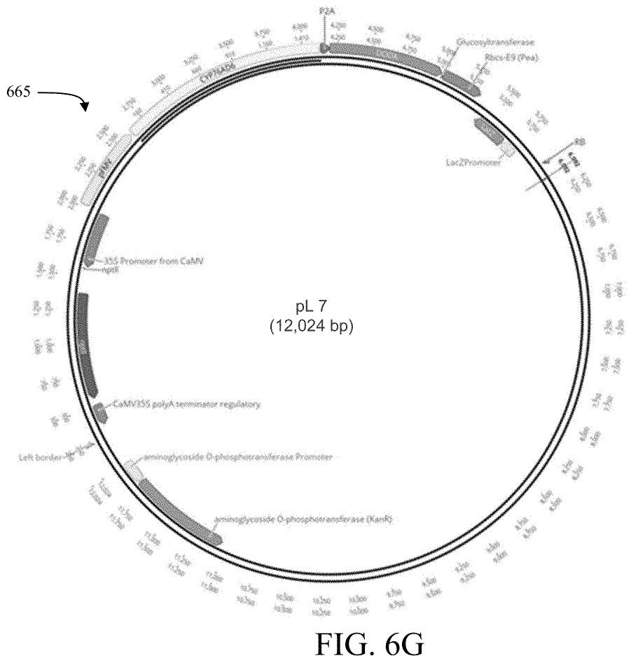

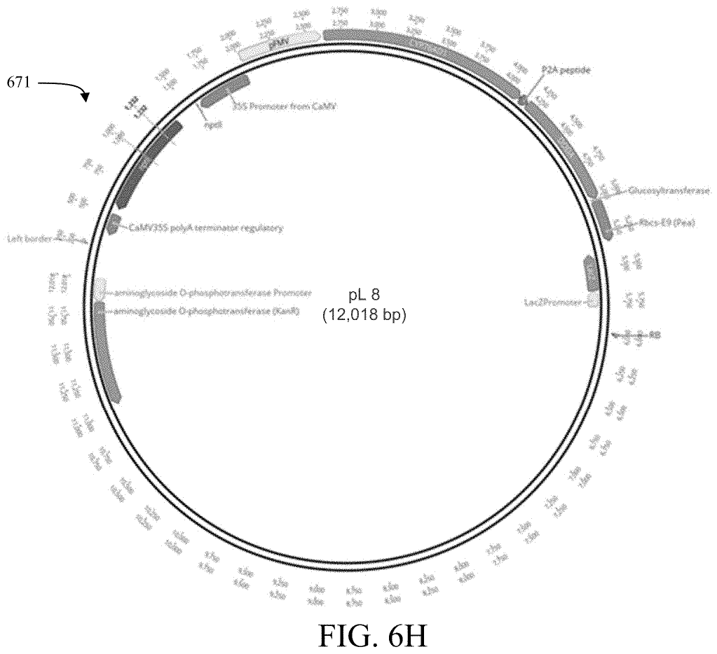

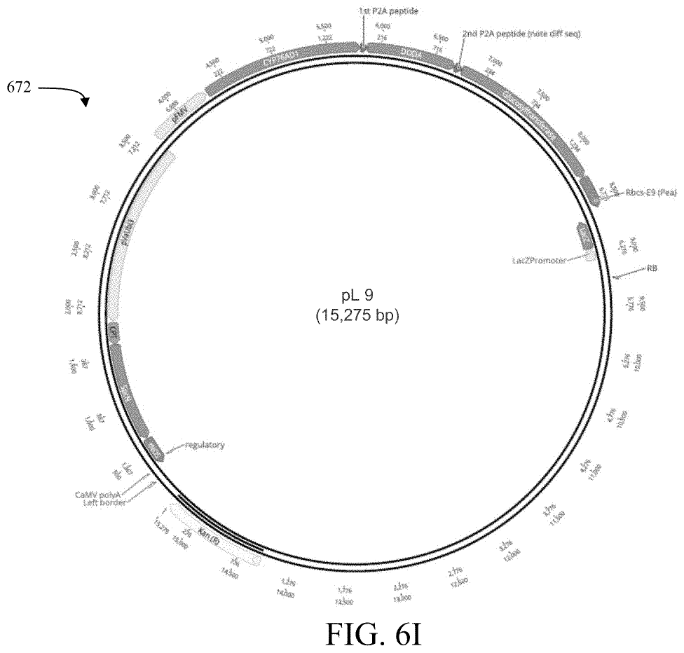

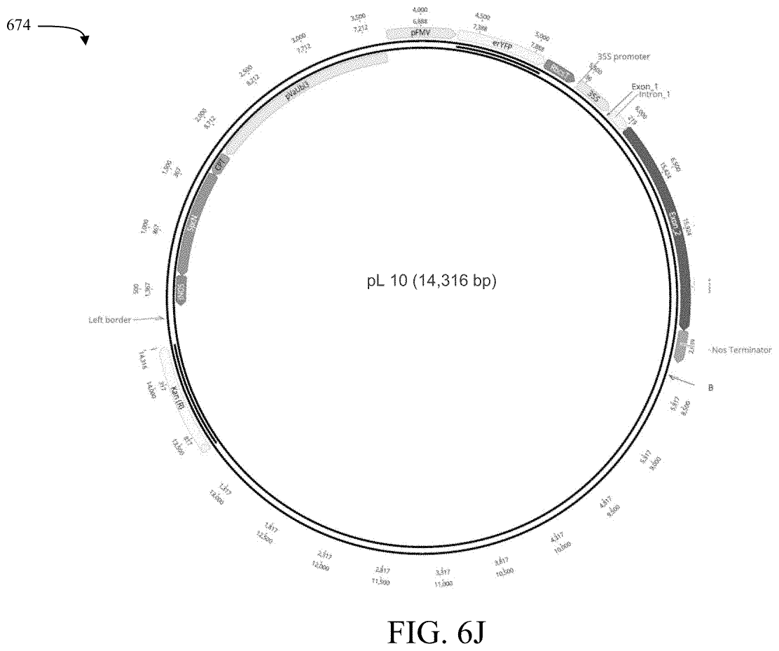

The PCM-derived compound can be isolated and purified from other components of the spent media. For example, a secreted protein and/or produced metabolite of interest can be isolated and purified from the spent media using recovery steps. In some embodiments, the PCM-derived compound is His-tagged, facilitating purification by binding, as discussed above. Factor Xa can be used to remove polyhistidine tags during protein affinity purification, such as illustrated by Xa cleavage Factor in the plasmid vectors 640, 650 of FIGS. 6B-6C. The Factor Xa can be removed by affinity-based capture after cleavage. In some embodiments, the PCM-derived compound is at least 60% pure, e.g., greater than 65%, 70%, 75%, 80%, 85%, 90%, 95%, or 99% pure.

To enhance recovery of the PCM-derived compound secreted by the PCM and/or metabolite of interest produced by the PCM-derived compound, an effective amount of a compound (e.g., protein, enzyme) stabilizing agent can be added to the growth medium. In general, compound stabilizing agents can include any substance conventionally employed during purification of a particular polypeptide to maintain compound concentration and activity by preventing compound degradation and denaturation, or any substance that blocks nonspecific interactions between the secreted compound and walls of the culture vessel. A compound stabilizing agent for use in PCM culture media should not support or encourage bacterium growth in the culture medium or be phytotoxic at the concentrations employed. Preferably, the compound stabilizing agent is used at levels that does not substantially reduce cell viability and integrity, protein expression or production, and growth and cell division. In addition, the compound stabilizing agent may not interfere with purification of the secreted recombinant compound. An “effective amount” of a compound stabilizing agent is an amount, when added to a given volume of a PCM culture medium, that significantly improves recovery of a secreted protein or produced metabolite from the medium, e.g., increasing compound recovery by a statistically significant amount. Preferably, recovery is increased by at least 20%, as compared with control medium that is otherwise identical except that it lacks the compound stabilizing agent. Compound stabilizing agents include without limitation preservatives and antimicrobials (e.g., benzalkonium chloride, glycerol, sodium azide, thymol), carbohydrates (e.g., sucrose, lactose, sorbitol, trehalose), antioxidants and reducing agents (e.g., Dithiothreitol, EDTA, 2-Mercaptoethanol), amino acids, derivatives of amino acids and albumin), and polymers (e.g., polyethylene glycol, polyvinylpyrrolidone).

The isolated and purified compound produced by the method 100 can be a protein substantially in its native fold and water soluble. In some embodiments, the isolated and purified protein is more than 50, 60, 70, 80, or 90% in its native fold. In some embodiments, the isolated and purified compound is more than 50, 60, 70, 80, or 90% water soluble. The fraction of properly folded protein can be increased by the addition of chemicals to the growth medium that reduce/oxidize disulfide bonds, and/or alter the general redox potential, and/or chemicals that alter solvent properties thus affecting protein conformation and aggregation. Exemplary additives include 2-mercaptoethanol or other disulfide reducing or oxidizing agents (e.g., DTT, TCEP, reduced and oxidized glutathione, cysteine, cystine, cysteamine, thioglycolate, etc.), which can be present in the media at a concentration of about 0.1 mM to 10 mM.

In some embodiments, the PCM-derived compound, such as a recombinant compound and/or metabolite of interest, is not secreted, or not fully secreted, by the PCM culture. The recombinant compound can accumulate in root tissue or cells of the PCM culture. When the PCM culture has grown to the desired stage, the culture or a portion thereof, can be harvested, and the PCM-derived compound and/or metabolite of interest isolated from the harvested material using conventional methods. For example, harvested tissue can be ground and the compound extracted with appropriate solvents. The crude product can then be purified in accordance with the nature of the product.

Purifying typically starts with a capture step to concentrate the core precursor compound, recombinant protein and/or metabolite of interest and remove any plant impurities that can be detrimental to protein yield, quality, and/or purification efficiency. For proteins intended for use in high purity applications (therapeutic, diagnostic uses), additional purification steps (polishing) can be implemented to remove residual host impurities and to ensure high product quality. Purification of PCM-derived proteins is dependent on protein properties and host cell impurities. The skilled artisan is capable of adapting strategies for purification employed with other heterologous expression systems (e.g., microbial systems). Thus, after conditioning and pretreatment, purification procedures can use techniques developed for biopharmaceutical products such as PCM-produced monoclonal antibodies (mAb), vaccines, hormones, etc. Purification of core precursor compound, recombinant compounds and/or metabolite of interest can include adsorption chromatography. A variety of resins are available. The skilled artisan can select an appropriate resin based on the production level of the core precursor compound, recombinant compound and/or metabolite of interest, spent media complexity and its effect on purification efficiency, product stability during processing, and removal methods for critical impurities. Resin selection is determined by core precursor compound, recombinant compound and/or metabolite of interest properties such as charge, hydrophobicity, and biospecificity. Selecting a resin based on the property most unique to the core precursor compound, recombinant compound and/or metabolite of interest compared to the other products of the PCM can improve purification efficiency by increasing binding capacity and/or product purity. Once suitable chromatography resin functionality is determined (cation/anion-exchange, affinity, hydrophobic), various resins with different particle sizes, surface areas, and resin backbones can be screened for purification efficiency at different binding conditions, such as pH and ionic strength. The core precursor compound, recombinant compound and/or metabolite of interest can be concentrated and partially purified by salt or polymeric precipitation as an intermediate purification step. Intermediate purification and polishing (further purification of eluted proteins from the capture step) can include a variety of orthogonal steps such as hydrophobic interaction chromatography (HIC), immobilized metal affinity chromatography (IMAC), ion-exchange chromatography, and ceramic hydroxyapatite chromatography, as dictated by the PCM-derived compound properties.

In some embodiments, the PCM generated can be used as a baseline to generate different recombinant compounds or other PCM-derived compounds and/or metabolites of interest. For example, and as further described herein, the PCM can include a plurality of core precursor compounds which are expressed at greater levels than the wild-type plant, and which can be used as building blocks to generate different recombinant or endogenous compounds by selectively triggering different bio-pathways. Example precursor compounds are described above.

In some embodiments, the PCM can be re-transformed using a nucleotide sequence encoding a PCM-derived compound, such as a recombinant compound, and which can cause another compound and/or metabolite to be produced by the PCM in response to the re-transformation.

FIG. 2 illustrates an example method for generating a PCM-derived compound using a PCM, consistent with the present disclosure. The method 200 can include an implementation of the method 100 of FIG. 1, in some embodiments. At 201, the method 200 includes contacting a plant part with a bacterium strain comprising an Ri plasmid or a Ti plasmid, a (first) nucleotide sequence encoding a gene that induces PCM formation (e.g., PCM gene), and a (second) nucleotide sequence encoding a PCM-derived compound. Although FIGS. 2-4 illustrate contacting a plant part with the bacterium strain to induce PCM formation and/or transforming a bacterium strain, embodiments are not so limited. In some embodiments, a wild-type bacterium strain can be used to transform the plant part to form a PCM, which can be enhanced by culturing the transformed plant part under growth conditions. In some embodiments, the plant part can be transformed using other transformation techniques which may not include use of a bacterium strain, as described above.

As previously described, the bacterium strain is prepared for infecting the plant part by introducing a nucleotide sequence encoding the PCM-derived compound (e.g., an endogenous or recombinant compound to the plant) into the bacterium strain (e.g., by electroporation) and culturing the transformed bacterium strain under conditions to select positively transformed cells. In some embodiments, the method 200 includes selecting the particular bacterium strain, as previously described.

In some embodiments, the bacterium strain used to transform and/or infect the plant part can include an Ri plasmid that includes the nucleotide sequence encoding the gene that induces PCM formation and can include the nucleotide sequence encoding the PCM-derived compound. For example, the Ri plasmid carries the gene that induces PCMs, sometimes herein referred to as “the PCM gene” for ease of reference, and a separate T-DNA carries the nucleotide sequence.

In some examples, the bacterium strain can be transformed to include a disarmed Ti plasmid, the nucleotide sequence encoding the gene that induces PCM formation, and the nucleotide sequence encoding the PCM-derived compound. In some examples, the bacterium strain can be transformed to include a disarmed Ri plasmid, the nucleotide sequence encoding the gene that induces PCM formation, and the nucleotide sequence encoding the PCM-derived compound. For example, a first T-DNA can carry the PCM gene and a second T-DNA can carry the nucleotide sequence encoding the PCM-derived compound.

In some embodiments, a vector or binary vector carrying the gene associated with the PCM-derived compound can include nucleic acid sequences encoding other gene editing reagents, such as rare-cutting endonucleases. The rare-cutting endonuclease(s) can be a transcription activator-like effector nuclease (TALE nuclease), a meganuclease, a zinc finger nuclease (ZFN), or a clustered regularly interspaced short palindromic repeats (CRISPR)/CRISPR-associated (Cas) nuclease reagent. In some examples, a rare-cutting endonuclease can be implemented as described in Baker, Nature Methods 9:23-26, 2012; Belahj et al., Plant Methods, 9:39, 2013; Gu et al., Nature, 435:1122-1125, 2005; Yang et al., Proc Natl Acad Sci USA, 103:10503-10508, 2006; Kay et al. Science, 318:648-651, 2007; Sugio et al., Proc Natl Acad Sci USA, 104:10720-10725, 2007; Römer et al. Science, 318:645-648, 2007; Schornack et al., J Plant Physiol, 163:256-272, 2006; and WO 2011/072246, each of which are incorporated herein in their entireties for their teachings.

In some examples, the vector or binary vector can include a transcription activator like effector nuclease (TALEN) sequence that encodes first and second TALE nucleases and binding domains to bind to target sites and cause a mutation at the target sites. The first TALE nuclease can generate a double stranded break at or near the first target site associated with a first binding domain and the second TALE nuclease can generate a double stranded break at or near the second target site associated with a second binding domain. In some embodiments, the first and second binding domains can be associated with a target gene. In some embodiments, the TALEN sequence can be co-delivered to the plant tissue with the secondary transgene to cause expression of the secondary transgene along with the PCM transgene.

As noted above, examples are not limited to TALENs and can include CRISPR/Cas systems (see, e.g., Belahj et al., Plant Methods, 9:39, 2013), among others or may not include the gene editing reagents. In some examples, a Cas9 endonuclease and a guide RNA can be used (either a complex between a CRISPR RNA [crRNA] and trans-activating crRNA [tracrRNA], or a synthetic fusion between the 3′ end of the crRNA and 5′end of the tracrRNA [sgRNA]). The guide RNA directs Cas9 binding and DNA cleavage to homologous sequences that are adjacent to a proto-spacer adjacent motif (PAM). Once at the target DNA sequence, Cas9 generates a DNA double-strand break at a position three nucleotides from the 3′ end of the crRNA targeting sequence. In some embodiments, this approach or other approaches, such as ZFN and/or meganucleases, can be used in addition to TALE nucleases to obtain modified plant parts.

At 203, the method 200 further includes culturing the plant part to enhance transformation of the plant part, induce PCM formation, and induce expression and/or production of the PCM-derived compound. In some embodiments, contacting and culturing the plant part includes transforming the plant part with the bacterium strain, inducing formation of PCM tissue from the transformed plant part, culturing the PCM tissue in a culture medium under the conditions.

In various embodiments, contacting and culturing (e.g., transforming) the plant part includes simultaneously introducing a first transgene and a second transgene to the plant part, and cultivating the plant part as transformed to generate PCM tissue. The first transgene can be associated with PCM formation, and the second transgene can be associated with the PCM-derived compound. In some embodiments, the first transgene is naturally occurring in the bacterium strain and the second transgene is non-naturally occurring and/or transgenic. In some embodiments, both the first transgene and the second transgene are non-naturally occurring and/or transgenic. In some embodiments, contacting and culturing the plant part with the bacterium strain can result in a transformation frequency of PCM formation of between 15 and 60 percent, and optionally, greater than 60 percent.

Embodiments, such as the method 100 of FIG. 1 and method 200 of FIG. 2, are not limited to simultaneously introducing a first transgene and a second transgene to the plant part. In some embodiments, the plant part is first transformed using the first transgene that induces the PCM phenotype to produce PCM tissue and the PCM tissue is isolated from wild-type tissue to generate the PCM culture and is retransformed using the second transgene associated with the PCM-derived compound, such as an endogenous or recombinant compound. For example, the first transformation can include a protocol involving a first bacterium strain as described above (e.g., culturing to form PCM tissue), and the second retransformation can include exposing the formed PCM tissue of the PCM culture to the second bacterium strain, such as 18r12. Other types of bacterium strains can be used as the second bacterium strain, including GV3101, AGL1, and EHA105.

As a specific example, contacting the plant part with the bacterium strain and culturing the plant part comprises contacting the plant part with a first bacterium strain comprising the (first) nucleotide sequence encoding the gene that induces PCM formation, and culturing the plant part to enhance PCM formation, such as under the above-described growth conditions. Followed by contacting the formed PCM tissue from the PCM with a second bacterium strain comprising the (second) nucleotide sequence encoding the PCM-derived compound, and culturing the PCM tissue to enhance production of the PCM-derived compound by the PCM.

In some embodiments, the first and/or second transformation can include other transformation techniques which may or may not include use of bacterium strain(s). For example, the first transformation can include contacting the plant part with a first nucleotide sequence encoding the gene that induces PCM formation, culturing the formed PCM tissue under growth conditions to enhance PCM formation, contacting PCM tissue of the PCM with a second nucleotide sequence encoding a PCM-derived compound (e.g., a recombinant compound or an endogenous compound), culturing the formed PCM tissue under growth conditions to enhance production or expression of the PCM-derived compound.

As described above, culturing the plant part can include inducing formation of PCM tissue from the plant part and culturing the PCM tissue in a culture medium under conditions for expression of the nucleotide sequence. For example, the method 200 can include screening new growth from the cultured plant part for PCM formation. For PCM induction, the plant part can be transferred into solid media with antibiotics two or three days after infection or co-cultivation, as previously described. The PCM can be induced from one week to over a month depending on the plant species. The decontaminated PCM tissue can be sub-cultured on hormone-free medium regularly (e.g., every one to two weeks).

In some embodiments, the new growth is screened to identify the transformed PCM tissue and the identified transformed PCM tissue is separated and sub-cultured in the culture medium under conditions for expression of the nucleotide sequence. The culture medium can include a liquid culture medium or a solid growth medium which is hormone-free. PCM tissue, as previously described, is isolated from photosynthetic wild-type tissue and, therefore, may not contain any remaining photosynthetic wild-type tissue. The absence of the added plant growth hormones can be used to select PCM tissue over wild type as the wild-type tissue can die in the absence of the growth hormone when grown in the dark. A second selection technique can be used, such as selection agent or reporter gene, as previously described.

FIG. 3 illustrates an example method for transforming a bacterium strain to comprise a sequence encoding a PCM-derived compound, consistent with the present disclosure. The method 300 can be combined with the method 100 and/or method 200 of FIGS. 1-2, in some embodiments.

At 305, the method 300 includes transforming a bacterium strain with the nucleotide sequence encoding the PCM-derived compound. In some embodiments, the bacterium strain is a wild-type bacterium strain including a Ri plasmid that carries the nucleotide sequence encoding a gene that induces PCM formation (e.g., PCM gene). In some embodiments, as described above, the bacterium strain is a wild-type bacterium strain that does not carry the gene that induces PCM formation, such as bacterium strain including a Ti plasmid. In such embodiments, transforming the bacterium strain can include transforming with both the nucleotide sequence encoding the PCM-derived compound and the gene that induces PCM formation.

The bacterium strain can be transformed using an expression construct. As used herein, an expression construct refers to or includes a nucleic acid sequence (e.g., DNA sequence) including vector(s) or binary vector(s) carrying gene(s). A vector or binary vector includes or refers to a DNA sequence that includes a transgene, sometimes referred to as “inserts”, and a backbone. The vector or binary vector can include an expression cassette that includes the transgene and a regulatory sequence to be expressed by a transformed plant cell. Successful transformation can result in the expression cassette directing plant cells to make the recombinant compound. As described above, embodiments are not limited use of a bacterium strain. In various embodiments, an expression construct, as described herein, can be used to transform plant cells of a plant part without use of a bacterium strain.

In some embodiments, the expression cassette includes the sequence encoding the PCM-derived compound, T-DNA border sequences, and a promoter. Expression cassettes typically include a promoter operably linked to a nucleotide sequence of interest (e.g., that encodes the recombinant compound or other transgene), which is optionally operably linked to termination signals and/or other regulatory elements. For example, the expression cassette can include TALEN T-DNA. The expression cassette can also include sequences required for proper translation of the nucleotide sequence, post-translational processing, localization and accumulation in a cellular compartment or tissue, or secretion into the PCM culture media. PCM-derived compounds, such as recombinant compounds, comprising signal peptides of plant origin (e.g., the N-terminal signal peptide from the tobacco PR1a protein or calreticulin) or signal peptides from eukaryotic secreted polypeptides, e.g., mammalian signal peptides, can be efficiently secreted through the plasma membrane and cell wall into the extracellular medium. In some embodiments, as further illustrated herein, the nucleotide sequence encoding the recombinant compound includes an N-terminal tag (e.g., signal to allow for purification of the protein after harvesting the roots). For example, in the case of membrane-spanning or -anchored proteins, the nucleotide construct can be prepared that modifies the N-terminus by replacing the membrane-spanning or membrane-anchoring domain with an N-terminal secretion signal sequence.

The expression cassette comprising the nucleotide sequence of interest can be chimeric, meaning that at least one of its components is heterologous with respect to at least one of its other components. The expression cassette can also be one which is naturally occurring or assembled entirely extracellularly (e.g., by recombinant cloning techniques). An expression cassette can be obtained by placing (or inserting) a promoter sequence upstream of an endogenous sequence, which thereby becomes functionally linked and controlled by the inserted promoter sequence.

In some embodiments, the bacterium strain is transformed using an expression construct that includes the vector or binary vector carrying a gene. The vector or binary vector can include a right T-DNA border sequence, a left T-DNA border sequence, the nucleotide sequence encoding the PCM gene and/or the PCM-derived compound (e.g., a recombinant or endogenous compound), and a promoter, such as including an expression cassette and vector backbone as previously described. An example expression construct can include a first vector that includes the nucleotide sequence encoding the PCM-derived compound and a second vector that includes the nucleotide sequence encoding the PCM gene. Each of the first and second vectors can include right and left T-DNA border sequences and a promoter. However embodiments are not so limited, and in some embodiments the bacterium strain already carries the PCM gene.

In some embodiments, the bacterium strain can be transformed in two separate transformation processes or using a vector carrying two or more transgenes (e.g., including multiple expression cassettes). For example, a first bacterium strain can including a wild-type bacterium strain that carries the PCM gene or that is transformed to carry the PCM gene, and a second bacterium strain that is transformed to carry the nucleotide sequence encoding the PCM-derived compound, which may be heterologous to the bacterium strain and/or to the plant of the plant part. For example, the method 300 can include transforming a first bacterium strain to carry the PCM gene and a second bacterium strain to carrying the nucleotide sequence encoding the PCM-derived compound.

In some embodiments, the promoter can include an inducible promoter, a strong promoter, or a root-tissue specific promoter. For example, the nucleotide sequence encoding the PCM-derived compound can be operably connected to the inducible promoter, strong promoter, or root-tissue specific promoter. In some embodiments, the promoter can include a constitutive promoter. An inducible promoter can be switched on and off, whereas a constitutive promoter can always be active. For example, the nucleotide sequence encoding the recombinant compound can be operably connected to an ubiquitin promoter (Ubi) or a 35S Cauliflower Mosaic Virus (CMV) promoter.

A promoter typically includes at least a core (basal) promoter, but can also include a control element. Such elements include upstream activation regions (UARs) and, optionally, other DNA sequences that affect transcription of a nucleic acid, which can include synthetic upstream elements. Factors for selecting a promoter to drive expression of the copy include efficiency, selectability, inducibility, desired expression level, and cell- or tissue-type specificity. The promoter can be one which preferentially expresses in root tissue or under certain conditions, e.g., is a root-tissue specific promoter. The promoter can be modulated by factors such as temperature, light or stress. For example, inducible promoters can be used to drive expression in response to external stimuli (e.g., exposure to an inducer). Suitable promoters include, but are not limited to, a light-inducible promoter from ssRUBISCO, MAS promoter, rice actin promoter, maize ubiquitin promoter, PR-I promoter, CZ19B1 promoter, milps promoter, CesA promoter, Gama-zein promoter, Glob-1, maize 15 kDa zein, 22 kDa zein, 27 kDa zein, δ-zein, waxy, shrunken 1, shrunken 2, globulin 1, pEMU promoter, maize H3 histone promoter, beta-estradiol, and dexamethasone-inducible promoters. Non-limiting examples of constitutive promoters include 35S promoter, such as 35S CMV promoter, 2x 35S promoter, nopaline synthase (NOS) promoter, ubiquitin-3(ubi3), among others.

A promoter for driving expression in the PCM culture can have strong transcriptional activity. A strong promoter drives expression of the PCM-derived compound encoding nucleic acid at a high level, or at about 1/10 transcripts to about 1/100 transcripts to about 1/1,000 transcripts. Enhancers can be utilized in combination with the promoter regions to increase transcription levels. When the PCM-derived compound is endogenous to the plant species, the expression cassette can be effective for achieving at least a 2-fold, 3-fold, 4-fold, 5-fold, 6-fold, 7-fold, 8-fold, 9-fold, or 10-fold or more increase in the level of production compared to the production level of the endogenous compound in the tissue in which it is normally found, as previously described.

The nucleotide sequence encoding the PCM-derived compound can include a DNA sequence derived from various organisms, including but not limited to, humans and other mammals and/or vertebrates, invertebrates, plants, sponges, bacteria, fungi, algae, archaebacteria, etc. Additionally, synthetic proteins, enzymes, and peptides are expressly contemplated, as are derivatives and analogs of any protein or compound. The compounds can be large or small, monomeric or multimeric, and have any type of utility. The DNA sequence can encode a recombinant compound having at least 70% (e.g., at least 75%, 80%, 85%, 90%, 95%, 97%, 98%, 99%, or 100%) sequence identity to the amino acid sequence of a corresponding protein of interest or production source for a metabolite of interest. In some cases, the DNA sequence has significant similarity and shared functional domains with the sequence encoding the protein of interest and/or production source for the metabolite of interest. The DNA sequence can be obtained from a related organism having a homologous, orthologous, or paralogous gene to a gene encoding the recombinant compound. In general, the methods for identifying conserved or similar DNA sequences and constructing recombinant genes encoding compounds related to the recombinant compound, optionally with various modifications for improved expression (e.g., codon optimized sequences), include conventional techniques in molecular biology. For example, PCR amplification or design and synthesis of overlapping, complementary synthetic oligonucleotides can be annealed and ligated together to yield a gene with convenient restriction sites for cloning, or subcloning from another already cloned source, or cloning from a library.

A number of nucleic acids can encode a PCM-derived compound having a particular amino acid sequence. Codons in the coding sequence for a given PCM-derived compound can be modified such that optimal expression in plants is obtained using appropriate codon bias tables. For example, at least some of the codons present a gene sequence that can be modified from a triplet code that is infrequently used in plants to a triplet code that is more common in plants.

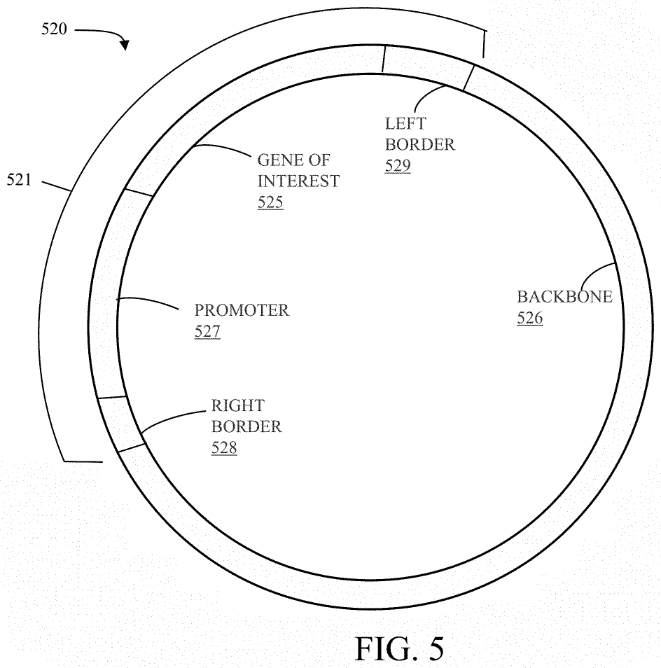

An example expression construct including a vector is illustrated by FIG. 5 and discussed further herein.

In some embodiments, the nucleotide sequence encodes a protein derived from an egg, such as albumin and/or ovalbumin. The egg-derived protein can be mammalian. The DNA sequence can encode an egg-derived protein having at least 70% (e.g., at least 75%, 80%, 85%, 90%, 95%, 97%, 98%, 99%, or 100%) sequence identity to the amino acid sequence of the corresponding wild-type egg protein. The percent identity between two amino acid sequences can be determined by aligning and comparing the sequences using analytical tools within the purview of the skilled artisan. However, embodiments are not so limited and can include heme-containing proteins, such as hemoglobin and globin-like proteins, among other proteins and production sources for metabolites, which can include or be used for or as antibodies, protein-based vaccines or therapeutics, nutritional proteins, dietary supplements, among other types of proteins, metabolites, and/or uses. For example, embodiments can be directed to deriving betalains, dopa or dopamine, flavonoids, stilbens, serpentines, flavone glycosides, cannabinoids, ginseng, indole, vitamins, total sterols, YFP, GFP or other reporter proteins, interleukins, growth factors, antigens, among others.

In some embodiments, the DNA sequence can include the sequence of a gene occurring in the wild-type plant or other organism, or a sequence having a percent identity that allows it to retain the function of the gene encoded product, such as a sequence with at least 90% identity. This sequence can be obtained from the organism or organism part or can be synthetically produced. The sequence can have at least 91%, 92%, 93%, 94%, 95%, 96%, 97%, 98%, 99% identity to the gene occurring in the wild-type organism. The sequence can be inserted at a different locus than that of the wild-type gene and be operably linked to a different promoter than the wild-type gene.

In some embodiments, the DNA sequence encoding a protease inhibitor for secretion is present in the transformed plant part. Co-secretion of the PCM-derived compound with a protease inhibitor into the plant growth medium can enhance stability and yield of the PCM-derived compound, such as a recombinant compound. The combined effect can involve the use of multiple separate nucleic acid constructs or transformation events. For example, multiple constructs as described above can be introduced into a plant part by the same or different methods, including the introduction of such a trait by the inclusion of two expression cassettes in a single vector, the simultaneous transformation of two expression constructs, or retransformation with a second expression construct.

At 307, the method 300 includes culturing the transformed bacterium strain. For example, the bacterium strain can be cultured in a rich media, such as Luria-Bertani (LB), a yeast extract peptone (YEP) media, and other rich media known in the art, or a minimal media, such as AB media (see media recipes below) and other minimal media known in art with appropriate antibiotics, as further described below. In some embodiments, a first bacterium strain and a second bacterium strain can be cultured.

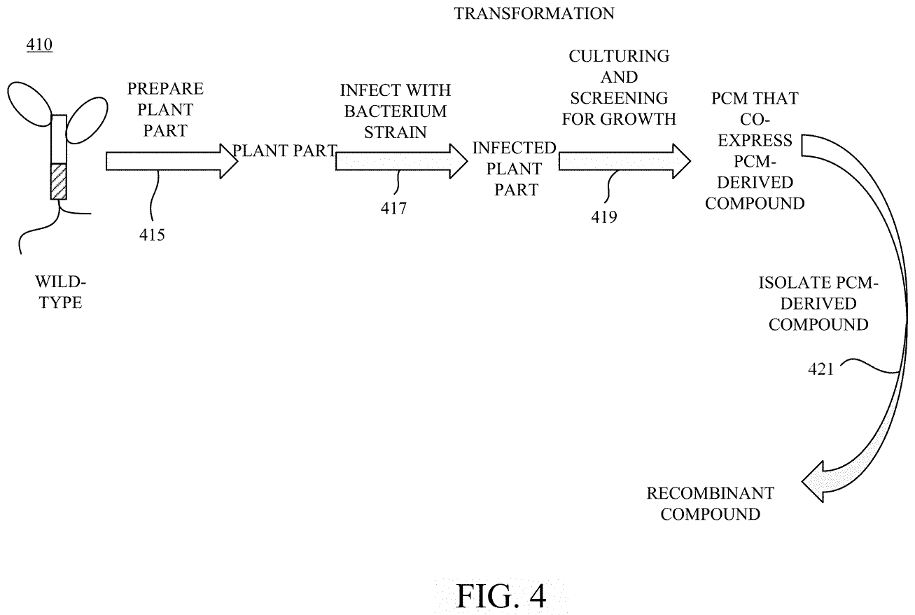

FIG. 4 illustrates an example method for transforming a plant part to induce PCM formation and production of a PCM-derived compound, consistent with the present disclosure. The method 410 can include an implementation of method 100 of FIG. 1 or method 200 of FIG. 2. At 415, the method 410 includes preparing a wild-type plant part, such as a cutting (e.g., hypocotyl segment), a seedling, a petiole, a node, a meristem, an internode, or a leaf excised from a host plant. In some embodiments, the host plant is Cannabaceae plant, however embodiments are not so limited and can be different to other plants such as other dicot plants.

At 417, the method 410 includes inoculating the wild-type plant part with a bacterium strain solution. The bacterium strain can be transformed to carry the nucleotide sequence encoding the PCM-derived compound, such as using an expression construct. Once such a construct is available, bacterium strains can be prepared carrying the vector and used to infect wild-type plant part(s). Selection of transgenic tissue (e.g., hairy root) using a plant selective agent (e.g., spectinomycin) can enrich the formation of high expressing transgenic PCM tissue (versus non-transgenic root tissue carrying only the Ri plasmid T-DNA or wild-type roots) and increase the expression of genome editing reagents in root tissues.

At 419, the method 410 includes culturing and screening the infected plant part. For example, the method 410 can includes transferring cuttings and/or whole seedlings infected with the bacterium strain to a medium for selection of transgenic tissue, e.g., PCM tissue. Other types of plant tissue can be used, such as a petiole, an internode, a node, a meristem, or a leaf. The degree of editing in the plant part can be directly related to the abundance of PCM-derived compound in tissues and can be tracked using various methods of PCM-derived compound detection. For instance, plant parts can be assayed for accumulation of the recombinant compound or other types of PCM-derived compounds in new PCM tissue. New PCM growth can be sampled for detection of PCM-derived compound using RT-PCR or western blot, respectively. PCM tissue growth positive for the PCM-derived compound can be screened for detection of edits using Illumina® amplicon sequencing of the target gene. PCM tissue growth positive for edits can be propagated either vegetatively or through seed to stabilize edits in individual plants, depending on the species.

At 421, the method 410 can include isolating the PCM-derived compound (e.g., a recombinant or endogenous compound) or a metabolite of interest, such as previously described and/or using a system as described below.

Various embodiments described above and including method 410 transforming and, optionally re-transforming a plant part to produce a PCM and a PCM-derived compound. The transformation and/or re-transformation can be accomplished by a wide variety of techniques, as described above by the examples provided for contacting the plant part with the nucleotide sequence.

Various embodiments of the present disclosure are directed to a non-naturally occurring plant part, such as a PCM and/or tissue generated by the methods of FIG. 1, FIG. 2 and/or FIG. 4.

Various embodiments of the present disclosure are directed to a PCM culture generated by the methods of FIG. 1, FIG. 2, and/or FIG. 4. In some embodiments, the PCM culture can be used for producing a PCM-derived compound, the PCM culture being induced from a plant part and a nucleotide sequence encoding the gene that induces PCM formation, wherein a cell of the PCM culture comprises the nucleotide sequence encoding the PCM-derived compound.

In other embodiments and/or in addition, the PCM culture comprises a plurality of core precursor compounds each expressed at a level of production that is greater than production in tissue of a wild-type plant. The plurality of core precursor compounds can be selected from alkaloids, triterpenes, sterols, fiber, carbohydrates, cannabinoids, flavonoids, fats, and combinations thereof. In some embodiments, the plurality of core precursor compounds can be selected from cannabisativine, anhydrocannabisativine, friedelin, epifriedelanol, beta-amyrin, beta-sitosterol, campesterol, stigmasterol, cellulose, NDF, ADF, crude fiber, carbohydrate, crude fat, THC, CBGA, CBDA, carvone, dihydrocarvone, p-hydroxy-trans-cinnamamide, lignans, choline, orientin, vitexin, isovitexin, quercetin, luteolin, kaempferol, apigenin, and combinations thereof. The PCM culture can be generated from a Cannabaceae plant, a Brassicaceae plant, or a Solanaceae plant, however embodiments are not so limited.

In some embodiments, a method can include identifying the bacterium strain from a plurality of bacterium strains. For example, a method can include transforming a plurality of plant parts with a plurality of bacterium strains to induce PCM formation, and optionally to induce production of a PCM-derived compound, and assessing transformation frequencies of the plurality of bacterium strains therefrom. In some embodiments, the plurality of plant parts are transformed with modified bacterium strains, such as bacterium strains carrying a nucleotide sequence encoding a gene that induces PCM formation and/or a nucleotide sequence encoding PCM-derived compound as described above, and cultured under conditions to enhance PCM formation. In some embodiments, the plurality of plant parts are transformed with wild-type bacterium strains, such as those that induce PCM formation. The plurality of bacterium strains can include a plurality of Rhizobium strains, a plurality of Agrobacterium strains, or combinations thereof. In some embodiments, the plurality of bacterium strains can include a plurality of R. Rhizogenes strains. In some embodiments, the plurality of bacterium strains can include a plurality of Agrobacterium tumefaciens strains. The method can further include selecting respective ones of the plurality of bacterium strains based on the transformation frequencies. For example, the respective ones bacterium strains with the highest transformation frequency or frequencies among the plurality of bacterium strains can be selected. In some examples, the selected bacterium strain is ATCC 43057, ATCC 43056, ATCC 13333, ATCC 15834, and/or K599. In some embodiments, the selected bacterium strain is ATCC 43057, ATCC 43056, ATCC 13333, or ATCC 15834. In some embodiments, the selected bacterium strain is ATCC 43057, ATCC 43056, or ATCC 13333.

In some embodiments, the PCM culture can be used to produce the compound of interest, e.g., core precursor compound, the recombinant compound or the metabolite of interest produced by the core precursor compound and/or recombinant compound. Various embodiments are directed to a system for producing the compound of interest from the PCM tissue. For example, the system can include a plurality of bioreactors in serial connection, wherein each bioreactor is inoculated with the PCM culture according to and/or obtained using any of above-described methods, and configured for growth and maintenance of the PCM culture in a culture medium.

In embodiments of the present disclosure, the PCM cultures are maintained in a bioreactor system. The PCM culture can be grown in a plastic sleeve reactor, a bubble reactor, a mist reactor, an airlift reactor, a liquid-dispersed reactor, or a bioreactor configured to generate micro- or nano-bubbles. A bioreactor can be any vessel adapted for receiving sterile growth media and enclosing the plant tissue therein. In some cases, a bioreactor is a flask (e.g., an Erlenmeyer flask).

A system comprising a plurality of bioreactors in serial connection for large scale production of the core precursor compound, recombinant compound and/or metabolite of interest is described herein. Each of the connected bioreactors can be structurally and operationally similar. Each bioreactor is configured with a growth chamber for housing the PCM culture, an inlet and an outlet. In some cases, one inlet of each bioreactor is connected to an air compressor configured to provide sterilized air to the PCM cultures. The air can be oxygen-enriched air. Substantially pure molecular oxygen can be provided. The bioreactors can include a separate inlet in fluid connection with a media supply system configured to provide growth media to the PCM culture. The connections can be made at the beginning of a growth/harvesting cycle (e.g., when the bioreactor is inoculated with the PCM culture) under anoxic conditions. The sterilized air and/or media can be provided continuously, or in predetermined pulses, during each culturing cycle. The system can be configured to remove excess air and/or waste gases by one of the outlets.

The bioreactor system can include holding tanks for media and additives. For example, micro elements, macro elements and vitamins, and additives such as antibiotics or fungicides can be held in different tanks. The system can include a mixer fed by a pump that delivers each component of the media at the desired relative proportions. The media can be delivered from the mixer by a delivery pipe having an aseptic connector.

The bioreactor system is configured to permit collection of the media for compound recovery. For example, the bioreactor can include a media outlet that can be closed by a valve. A portion of spent media can be removed from each bioreactor by opening the valve before or as fresh media is supplied to the bioreactor. The removal can be achieved under gravity, whereby the spent media flows into a conduit connected to each of the bioreactors to pool spent media. In addition, the system can permit media to be harvested from each bioreactor separately. The conduit can include a sample port that allows for collection of smaller samples of the spent media for detecting secretion of the PCM-derived compound. The conduit can be configured for conditioning and pretreatment of the spent media. In some embodiments, the conduit is in fluid connection with components for capture of the core precursor, protein or produce metabolite of interest (e.g., ion-exchange columns). The system can be configured for continuous recovery of the core precursor, protein and/or produced metabolite once the PCM culture achieves a steady state of core precursor or protein secretion or metabolite production. In some embodiments, the spent media can flow into a removable recovery tank for batch-wise purification of the core precursor, protein or produced metabolite. The recovery tank can be removed from the bioreactor system periodically and the contents decanted for isolation and purification of the secreted protein or produced metabolite.

The operation of the bioreactor system can be controlled by circuitry, such as a processor and/or computer that includes a processor and memory. The circuitry can be configured to control parameters such as temperature, amount and timing of air entering the bioreactors and/or exit of waste gases, amount and timing of the addition of culture medium, and/or amount of light. The circuitry can be connected to the conduit or a sample port. The circuitry can control an automated sampler and/or media harvester for removing portions of the spent media for testing and/or recovery. The circuitry can also optionally be connected to an analyzer to provide feedback for operation of the circuitry.

FIG. 5 illustrates example expression construct for delivery of a gene encoding a recombinant compound, consistent with the present disclosure. The example expression construct 520 is or includes a vector containing an expression cassette 521 and a vector backbone 526. The expression cassette 521 includes a transgene that causes expression of the recombinant compound. The transgene of the expression cassette 521 includes a gene of interest 525, a promoter 527, a left border 529, and a right border 528. The expression construct 520 and/or expression cassette 521 can include various additional components, such as TALE sequences, a selection agent, a terminator, and an additional expression cassette, among other components, such as signaling peptides, compound markers, and/or compound purification tags.

As used herein, a “plant” refers to any organism of the kingdom Plantae. In some embodiments, the plant or plant part is or is from a dicotyledonous plant. Non-limiting example plants can be from the families of Cannabaceae, Brassicaceae, Fabaceae, Poaceae, Solanaceae, Apiaceae, Malvaceae, and Asteraceae, among other plant families. In some embodiments, the plant includes a plant species selected from the families of Cannabaceae, Brassicaceae, Solanaceae, Fabaceae, and Apiaceae. In some embodiments, the plant includes a plant selected from the families of Cannabaceae, Brassicaceae, and Solanaceae. In some embodiments, the plant is selected from the families of Cannabaceae and Solanaceae. Non-limiting example plants include but are not limited to Cannabis sativa, Cannabis indica, Cannabis ruderalis, Humulus, Celtis, Alphananthe, Chaetachme, Gironniera, Lozanella, Parasponia, Pteroceltis, Trema, Glycine max, Phaseolus, Pisum sativum, Civer aretinum, Medicago sativa, Arachis hypogaea, Ceratonia siliqua, Glycyrrhiza glabra, Avena sativa, Zea mays, Triticum aestivum, Oryza sativa, Oryza glaberrima, Hordeum vulgare, Eleusine coracana, Panicum miliaceum, Daucus carota, Solanum lycopersicon, Solanum aviculare, Solanum nigrum, Catharanthus roseus, Panax quinquefolius, Nicotiana tabacum, Atropa belladoma, Thlaspi caerulescens, Brassica napus, Brassica juncea, Ipomoea batatas, Helianthus annuus, and Gossypium plants or plant parts, among other plants or plant parts.

In some embodiments, the plant includes a Cannabaceae plant or plant part. As used herein, Cannabaceae refers to a plant of the family Cannabaceae. For example, the Cannabaceae plant or plant part can include a plant or plant part that belongs to the genus of Cannabis, sometimes referred to as a Cannabis plant or plant part, and which includes Cannabis sativa, Cannabis indica, and Cannabis ruderalis. However, embodiments are not so limited, and the Cannabaceae plant or plant part can include Humulus (e.g., hops), Celtis, Alphananthe, Chaetachme, Gironniera, Lozanella, Parasponia, Pteroceltis, and/or Trema plants or plant parts, among other plants or plant parts.

In some embodiments, the plant includes a Brassicaceae plant or plant part. As used herein, Brassicaceae refers to a plant of the family Brassicaceae. For example, the Brassicacee plant or plant part can belong to the genus of Draba, Erysium, Lepidium, Cardamine, or Alyssum, among others. In some embodiments, the Brassicacee plant or plant part includes Brassica oleracea (e.g., broccoli, cabbage, cauliflower, kale. collards), Brassica rapa (e.g., turnip, Chinese cabbage, etc.), Brassica napus, Raphanus sativus (e.g., common radish), Armoracia rusticana (e.g., horseradish), or Arabidopsis thaliana (e.g., thale cress), among other plants.

In some embodiments, the plant includes a Solanaceae plant or plant part. As used herein, Solanaceae refers to a plant of the family Solanaceae. For example, the Solanaceae plant or plant can belong to the genus of Solanum, such as Solanum tuberosum, Solanum dulcamara, Solanum lycopersicum, Solanum melongena, Solanum aethiopicum, Solanum quitoense, Solanum torvum, Solanum muricatum, Solanum betaceum, Solanum lycocarpum, and Solanum scabrum, among others. However, embodiments are not so limited, and the Solanaceae plant or plant part can include Lycianthes, Cestrum, Nolana, Physalis, Lycium, Nicotiana, Brunfelsia, Sessea, Vestia, Reyesia, Salpiglossis, Coeloneurum, Goetzea, Anthocercis, Cypanthera, Benthamiella, Brunfelsia, Calibrachoa, Leptoglossis, Nierembergia, Petunia, Schizanthus, Schwenckia, Iochroma, Chamaersaracha, or Jaltomata, among others.

In some embodiments, the plant includes a Fabaceae plant or plant part. As used herein, Fabaceae refers to a plant of the family Fabaceae. For example, the Fabaceae plant or plant can belong to the genus of Glycine, such as Glycine max (e.g., soybean), Glycine soja, Glycine albicans, Glycine curvata, or Glycine pemota, among others. However, embodiments are not so limited, and the Fabaceae plant or plant part can include Phaselous, Pisum (e.g., Pisum sativum), Cicer (e.g., Cicer arietinum), Medicago (e.g., Medicago sativa), Arachis (e.g., Arachis hypogaea), Ceratonia (e.g., Ceratonia siliqua), Glycyrrhiza (e.g., Glycyrrihiza glabra), Cytisus (e.g., Cytisus scoparus), Robinia (e.g., Robinia pseudoacacia), Ulex (e.g., Ulex eropaeus), Pueraria (e.g., Pueraria montant), or Lupinus, among others.

In some embodiments, the plant includes an Apiaceae plant or plant part. As used herein, Apiaceae refers to a plant of the family Apiaceae. In some embodiments, Apiaceae plant or plant part can belong to the of Daucus, Pastinaca, Petraselinum, Coriandrum, Anethum, Foeniculum, Cuminum, Carum, Anthriscus, Apium, Arracacia, Ferula, Pimpinelia, or Myrrhis, among others. In some embodiments, the Apiaceae plant or plant part includes Daucus caroti, Pastinaca sativa, Petroselinum crispum, Coriandrum sativum, Anethurn graveolens, Foeniculum vulgare, Cuminum cyminum, Carum carvi, Anthriscus cereolium, Apium graveolens, Arracacia xanthorrhiza, Ferula asafetida, Ferula gummosa, Pimpinella ansium, Myrrhis odorata, or Levisticum officinale, among others.

The term “plant” generally refers to whole plants, but when “plant” is used as an adjective, refers to any substance which is present in, obtained from, derived from, or related to a plant, such as plant organs (e.g., leaves, stems, roots, flowers), single cells (e.g., pollen), seeds, plant cells including tissue cultured cells, products produced from the plant. The term “plant part” refers to plant tissues or organs which are obtained from a whole plant. Plant parts include vegetative structures (for example, leaves, stems), roots, floral organs/structures, seed (including embryo, endosperm, and seed coat), plant tissue (for example, vascular tissue, ground tissue, and the like), cells and progeny of the same. The term “plant cell” refers to a cell obtained from a plant, and includes protoplasts or other cells derived from plants, gamete-producing cells, and cells which regenerate into whole plants. Plant cells can be cells in culture. “Plant tissue” means differentiated tissue in a plant or obtained from a plant (“explant”) or undifferentiated tissue derived from immature or mature embryos, seeds, roots, shoots, fruits, pollen, and various forms of aggregations of plant cells in culture, such as calli. Plant tissues in or from seeds, such as seeds, including a seed coat or testa, storage cotyledon, and embryo. A “plant clone” is a plant or plant part produced via well-known plant cloning processes. A plurality of clones can be produced from a single individual plant through asexual reproduction. A “plant line” or “bacterium line” (or strain) refers to a particular strain of the plant or bacteria.

The phrase “protein of interest”, “metabolite of interest”, or “compound of interest” corresponds to any protein, production source, or metabolite that can be produced by the method according to the present disclosure. The protein, production source, and/or metabolite of interest can be endogenous to the plant, or exogenous. In a case where the protein, production source, or metabolite of interest is endogenous to the plant, e.g., produced naturally by the plant, the protein, production source, and/or metabolite of interest is overproduced with respect to an untransformed plant.

The skilled artisan would recognize that various terminology as used in the Specification (including claims) connote a plain meaning in the art unless otherwise indicated. The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of” and “consisting essentially of,” the embodiments or elements presented herein, whether explicitly set forth or not. The various values, percentages, and ranges provided herein include the stated range and any value or sub-range within the stated range. Furthermore, when “about” is utilized to describe a value or percentage this includes, refers to, and/or encompasses variations (up to +/−10%) from the stated value or percentage.

The singular forms “a”, “and” and “the” include plural references unless the context clearly dictates otherwise. For example, singular forms of “a PCM gene” or a “gene that induces PCM formation”, as used herein, includes a single PCM gene and a plurality of PCM genes in different embodiments (e.g., one or more PCM genes). As other non-limiting examples, “a nucleotide sequence”, “a vector”, “an expression construct”, “an expression cassette”, “a plant part”, “a culture medium”, “a core precursor compound”, “a PCM-derived compound”, “a recombinant compound”, “a transgene”, “a PCM culture”, “a bacterium strain”, among others singular forms of elements or components includes a singular form and a plurality form of the element or component, such as one or more nucleotide sequences, one or more vectors, one or more expression constructs, one or more expression cassettes, one or more a plant parts, one or more culture mediums, one or more core precursor compounds, one or more PCM-derived compounds, one or more recombinant compounds, one or more transgene, one or more PCM cultures, one or more bacterium strains, among others.

Various embodiments are implemented in accordance with the underlying provisional applications and PCT Application: (i) U.S. Provisional Application No. 63/162,702, filed on Mar. 18, 2021, and entitled “Expressing Recombinant Compounds Using Cannabaceae Hairy Roots”; (ii) U.S. Provisional Application No. 63/304,850, filed on Jan. 31, 2022, and entitled “Expressing Recombinant Compounds Using Plant Cell Matrices”; (iii) U.S. Provisional Application No. 63/184,487, filed on May 5, 2021, and entitled “Expressing Albumin Using Hairy Roots”; (iv) U.S. Provisional Application No. 63/240,660, filed on Sep. 3, 2021, and entitled “Producing Betalains Using Hairy Roots”; and PCT Application Serial Number PCT/US2022/021014, filed on Mar. 18, 2022, and entitled “Plant Cell Matrices and Methods Thereof”, and to each of which benefit is claimed and each are fully incorporated herein by reference. For instance, embodiments herein and/or in the provisional and/or PCT applications can be combined in varying degrees (including wholly). Embodiments discussed in the provisional applications are not intended, in any way, to be limiting to the overall technical disclosure, or to any part of the claimed invention unless specifically noted.

Based upon the above discussion and illustrations, those skilled in the art will recognize that various modifications and changes can be made to the embodiments without strictly following the exemplary embodiments and applications illustrated and described herein. For example, methods as exemplified in the Figures can involve steps carried out in various orders, with aspects of the embodiments herein retained, or can involve fewer or more steps. Such modifications do not depart from the scope of various aspects of the disclosure, including aspects set forth in the claims.

### Experimental Embodiments

As further illustrated below in connection with the experimental embodiments, plant parts were transformed to induce PCM formation and produce a PCM-derived compound, such as a recombinant compound. Different experiments were conducted to illustrate successfully transforming a bacterium strain and/or transforming a plant part with the nucleotide sequences(s) that encode the PCM gene and/or a PCM-derived compound, such as a recombinant compound. The transformed plant parts exhibited PCM tissue which produced a compound of interest. Further experiments were conducted to transform the plant part to induce PCM formation and to re-transform the PCM cells to produce a recombinant compound, such as albumin and/or betalain. Example constructs and sequences used to experimental embodiments include the nucleotide sequences set forth in SEQ ID NOs: 1-95. SEQ ID NOs: 1-95 are each synthetic DNA.

FIGS. 6A-6J illustrate example binary vectors for delivery of a sequence encoding a recombinant compound, consistent with the present disclosure. Each binary vector for bacterium strain transformation (e.g., R. rhizogenes transformation) contains a right T-DNA border sequence and a left T-DNA border sequence, to allow the bacterium strain to deliver the DNA into the plant cells. The binary vectors are plasmids and can be referred to as plasmid vectors. At least some of the binary vectors further include a DNA sequence coding for a recombinant compound, which is codon optimized according to the codon bias used by the target Cannabaceae species (such as, Cannabis sativa, Cannabis indica, and Cannabis ruderalis), and cloned in binary vectors, are under the regulation of a strong promoter (Ubi3) or another promoter, such as a 35S cauliflower mosaic viral promoter, and a terminator. Constitutive promoters and root specific promoters are selected for tissue-specific approaches.

FIG. 6A illustrates an example plasmid vector 630 that includes a DNA sequence coding for YFP protein. The plasmid vector 630 can be used as a control. The plasmid vector 630 contains the gene encoding enhanced yellow fluorescent protein (eYFP) driven by the Figwort mosaic virus (pFMV) promoter along with the spectinomycin (SPCN) gene which confers resistance to spectinomycin in the transformed plant cells driven by the Ubi3 promoter. These two gene cassettes are flanked by the left (LB) and right border (RB) T-DNA sequences allowing for transfer of the entire sequence or transgene into the plant cells by the bacterium strain of R. rhizogenes. The plasmid backbone also contains a kanamycin resistance (KanR) gene which allows for selection and maintenance of the plasmid within the R. rhizogenes strain. The Cannabaceae plant parts (e.g., Cannabis) were transformed using the R. rhizogenes strain A4 (ATCC43057). The plasmid vector 630 sequence comprises SEQ ID NO: 1. Further identified is the sequence of YFP (SEQ ID NO: 2), KanR gene (SEQ ID NO: 3), pFMV (SEQ ID NO: 4), Ubi3 promoter (SEQ ID NO: 5), SPCN gene (SEQ ID NO: 6), left border (SEQ ID NO: 7), right border (SEQ ID NO: 8), a NOS terminator (SEQ ID NO: 9) and Rbcs-E9 (SEQ ID NO: 10). Rbcs-E9 is a transcriptional terminator element derived from Pisum sativa.

FIG. 6B illustrates an example plasmid vector 640 that includes a DNA sequence coding for albumin or ovalbumin. The plasmid vector 640 contains the gene encoding albumin derived from chicken eggs or ovalbumin driven by the Ubi3 promoter. The gene cassettes are flanked by the LB and RB T-DNA sequences allowing for transfer of the entire sequence or transgene into the plant cells by the bacterium strain of R. rhizogenes. The plasmid backbone also contains the KanR gene for selection and maintenance of the plasmid within the R. rhizogenes strain and LacZ gene and LacZ promoter used as a selectable marker. The ovalbumin has a histidine tag on the N-terminal end to allow for purification of the protein after harvesting of the roots using an affinity column. The Cannabaceae plant parts (e.g., Cannabis) were transformed using the R. rhizogenes strain A4 (ATCC43057). The plasmid vector 640 sequence comprises SEQ ID NO: 11. Further identified is the sequence of ovalbumin (SEQ ID NO:12), KanR gene (SEQ ID NO: 3), Ubi3 promoter (SEQ ID NO: 5), LacZ gene (SEQ ID NO: 13), LacZ promoter (SEQ ID NO: 14), Tobacco Mosaic Virus promoter (pTMV) (SEQ ID NO: 15), histidine tag (SEQ ID NO: 16), spacer (SEQ ID NO: 17), Xa cleavage Factor (SEQ ID NO: 18), left border (SEQ ID NO: 7), right border (SEQ ID NO: 8), and a NOS terminator (SEQ ID NO: 9).

FIG. 6C illustrates an example plasmid vector 650 that includes a DNA sequence coding for ovalbumin. The plasmid vector 650 contains the gene encoding ovalbumin driven by 35S CMV promoter to drive overexpression. The gene cassettes are flanked by the LB and RB T-DNA sequences allowing for transfer of the entire sequence or transgene into the plant cells by the bacterium strain of R. rhizogenes. The plasmid backbone contains the KanR gene for selection and maintenance of the plasmid within the R. rhizogenes strain and a LacZ gene and LacZ promoter used as a selectable marker. The ovalbumin has a histidine tag on the N-terminal end to allow for purification of the protein after harvesting of the roots using an affinity column. The Cannabaceae plant parts (e.g., Cannabis) were transformed using the R. rhizogenes strain A4 (ATCC43057). The plasmid vector 650 sequence comprises SEQ ID NO: 19. Further identified is the sequence of ovalbumin (SEQ ID NO: 12), pTMV (SEQ ID NO: 15), 35S CMV promoter (SEQ ID NO: 20), LacZ gene (SEQ ID NO: 13), LacZ promoter (SEQ ID NO: 14), histidine tag (SEQ ID NO: 16), spacer (SEQ ID NO: 17), Xa cleavage Factor (SEQ ID NO: 18), left border (SEQ ID NO: 7), right border (SEQ ID NO: 8), and NOS terminator (SEQ ID NO: 9).

FIGS. 6D-6I illustrate example expression constructs for delivery of a sequence encoding an enzyme, consistent with the present disclosure. Each expression construct for bacterium strain transformation (e.g., R. rhizogenes transformation) contains a right T-DNA border sequence and a left T-DNA border sequence, to allow the bacterium strain to deliver the DNA into the plant cells. The expression constructs further include a betalain cassette, such as a DNA sequence coding an enzyme for producing betalain from tyrosine, which is codon optimized according to the codon bias used by the target, and cloned in binary vectors, are under the regulation of a promoter, such as a FMV promoter, and a terminator. The enzymes include CYP76AD1, CYP76AD6, DODA, and/or glucosyltransferase, and various combinations thereof. The enzymes are separated by 2A self-cleaving sequences, such a sequences encoding F2A or P2A. For example, a first 2A self-cleaving sequence links CYP76AD1 to DODA and a second 2A self-cleaving sequence links DODA to glucosyltransferase. Constitutive promoters and root specific promoters are selected for tissue-specific approaches. The expression constructs further include additional cassettes, such as a plant selectable marker cassette, a LacZ cassette, and a bacterial selection maker cassette. The additional cassettes are oriented in reverse on the plasmid as compared to the betalain cassette.

FIG. 6D illustrates a plasmid vector 652 at includes a DNA sequence coding for a plurality of enzymes to produce betalain (e.g., betanidin). The plasmid vector 652 contains the gene encoding the enzymes CYP76AD1, DODA, and glucosyltransferase driven by a FMV promoter, with CYP76AD1 linked to DODA and DODA linked to glucosyltransferase by 2A self-cleaving peptides F2A. The plasmid vector 652 further includes a plant selectable marker cassette and a LacZ cassette, which are in reverse orientation on the plasmid vector 652, as further described below. The plant selectable marker cassette encodes a selection marker that when expressed, confers resistance to a selection agent (e.g., bacteria or other toxic substances) for selection of transformed plant cells, a promoter, and a terminator. The LacZ cassette encodes a LacZ gene and LacZ promoter used as a selectable marker. The gene cassettes are flanked by the LB and RB T-DNA sequences allowing for transfer of the entire sequence or transgene into the plant cells by the bacterium strain of R. rhizogenes. The plasmid backbone also contains a bacterial selection marker cassette that encodes the KanR gene for selection and maintenance of the plasmid within the R. rhizogenes strain, and which is in reverse orientation on the plasmid vector 652. The Cannabaceae plant parts (e.g., Cannabis) were transformed using the R. rhizogenes strain A4 (ATCC43057). The plasmid vector 652 sequence comprises SEQ ID NO: 21. Further identified is the sequence of the betalain cassette (SEQ ID NO: 22), the plant selectable marker cassette (SEQ ID NO: 30), the T-DNA borders of the RB (SEQ ID NO: 35) and LB (SEQ ID NO: 36), the LacZ cassette (SEQ ID NO: 37) and the bacterial selection marker cassette (SEQ ID NO: 40). The betalain cassette (SEQ ID NO: 22) encodes the FMV promoter (SEQ ID NO: 23), CYP76AD1 (SEQ ID NO: 24), F2A 1 (SEQ ID NO: 25), DODA (SEQ ID NO: 26), F2A 2 (SEQ ID NO: 27), glucosyltransferase (SEQ ID NO: 28), and a ribulose bisphosphate carboxylase (rbcS) terminator (SEQ ID NO: 29). The plant selectable marker cassette (SEQ ID NO: 30) encodes a 35S promoter (SEQ ID NO: 31), an ST LS1 nptll intron (SEQ ID NO: 32), a nptll exon (SEQ ID NO: 33), and a 35S terminator (SEQ ID NO: 34). The LacZ cassette (SEQ ID NO: 37) encodes the LacZ promoter (SEQ ID NO: 38) and LacZ gene (SEQ ID NO: 39). The bacterial selection marker cassette (SEQ ID NO: 40) encodes the KanR promoter (SEQ ID NO: 41) and KanR gene (SEQ ID NO: 42). FIG. 6E illustrates an example plasmid vector 657 that includes a DNA sequence coding for a plurality of enzymes to produce betalain (e.g., betanidin). The plasmid vector 657 contains the gene encoding the enzymes CYP76AD1, DODA, and glucosyltransferase driven by a FMV promoter, with CYP76AD1 linked to DODA and DODA linked to glucosyltransferase by 2A self-cleaving peptides P2A. The plasmid vector 657 further includes a plant selectable marker cassette and a LacZ cassette, which are in reverse orientation on the plasmid vector 657 and as described by plasmid vector 652, the features of which are not repeated. The gene cassettes are flanked by the LB and RB T-DNA sequences, as described above. The plasmid backbone also contains a bacterial selection marker cassette that encodes the KanR gene, and which is in reverse orientation on the plasmid vector 657. The Cannabaceae plant parts (e.g., Cannabis) were transformed using the R. rhizogenes strain A4 (ATCC43057). The plasmid vector 657 sequence comprises SEQ ID NO: 43. Further identified is the sequence of the betalain cassette (SEQ ID NO: 44), the plant selectable marker cassette (SEQ ID NO: 30), the T-DNA borders of the RB (SEQ ID NO: 35) and LB (SEQ ID NO: 36), the LacZ cassette (SEQ ID NO: 37) and the bacterial selection marker cassette (SEQ ID NO: 40). The betalain cassette (SEQ ID NO: 44) encodes the FMV promoter (SEQ ID NO: 23), CYP76AD1 (SEQ ID NO: 24), P2A 1 (SEQ ID NO: 45), DODA (SEQ ID NO: 26), P2A 2 (SEQ ID NO: 46), glucosyltransferase (SEQ ID NO: 28), and a rbcS terminator (SEQ ID NO: 29). The plant selectable marker cassette (SEQ ID NO: 30) encodes a 35S promoter (SEQ ID NO: 31), an ST LS1 nptll intron (SEQ ID NO: 32), a nptll exon (SEQ ID NO: 33), and a 35S terminator (SEQ ID NO: 34). The LacZ cassette (SEQ ID NO: 37) encodes the LacZ promoter (SEQ ID NO: 38) and LacZ gene (SEQ ID NO: 39). The bacterial selection marker cassette (SEQ ID NO: 40) encodes the KanR promoter (SEQ ID NO: 41) and KanR gene (SEQ ID NO: 42).

FIG. 6F illustrates an example plasmid vector 660 that includes a DNA sequence coding for a plurality of enzymes to produce betalain (e.g., betanidin). The plasmid vector 660 contains the gene encoding the enzymes CYP76AD1, DODA, and glucosyltransferase driven by a FMV promoter, with CYP76AD1 linked to DODA and DODA linked to glucosyltransferase by 2A self-cleaving peptides P2A. The plasmid vector 660 further includes a plant selectable marker cassette and a LacZ cassette, which are in reverse orientation on the plasmid vector 660. In the particular plasmid vector 660, the plant selectable marker cassette includes the CP4 gene. The gene cassettes are flanked by the LB and RB T-DNA sequences, as described above. The plasmid backbone also contains a bacterial selection marker cassette that encodes the KanR gene, and which is in reverse orientation on the plasmid vector 660. The Cannabaceae plant parts (e.g., Cannabis) were transformed using the R. rhizogenes strain A4 (ATCC43057). The plasmid vector 660 sequence comprises SEQ ID NO: 47. Further identified is the sequence of the betalain cassette (SEQ ID NO: 48), the plant selectable marker cassette (SEQ ID NO: 49), the T-DNA borders of the RB (SEQ ID NO: 35) and LB (SEQ ID NO: 36), the LacZ cassette (SEQ ID NO: 37) and the bacterial selection marker cassette (SEQ ID NO: 40). The betalain cassette (SEQ ID NO: 48) encodes the FMV promoter (SEQ ID NO: 23), CYP76AD1 (SEQ ID NO: 24), P2A 1 (SEQ ID NO: 45), DODA (SEQ ID NO: 26), P2A 2 (SEQ ID NO: 46), glucosyltransferase (SEQ ID NO: 28), and a rbcS terminator (SEQ ID NO: 29). The plant selectable marker cassette (SEQ ID NO: 49) encodes a VuUbi promoter (SEQ ID NO: 50), a chloroplast transit peptide (SEQ ID NO: 51), a Cp4 gene (SEQ ID NO: 52), and a NOS terminator (SEQ ID NO: 53). The LacZ cassette (SEQ ID NO: 37) encodes the LacZ promoter (SEQ ID NO: 38) and LacZ gene (SEQ ID NO: 39). The bacterial selection marker cassette (SEQ ID NO: 40) encodes the KanR promoter (SEQ ID NO: 41) and KanR gene (SEQ ID NO: 42).

FIG. 6G illustrates an example plasmid vector 665 that includes a DNA sequence coding for a plurality of enzymes to produce betalains (e.g., betaxanthin). The plasmid vector 665 contains the gene encoding the enzymes CYP76AD6 and DODA driven by a FMV promoter, with CYP76AD6 linked to DODA by a 2A self-cleaving peptide P2A. The plasmid vector 665 further includes a plant selectable marker cassette and a LacZ cassette, which are in reverse orientation on the plasmid vector 665 and as described by plasmid vector 652, the features of which are not repeated. The gene cassettes are flanked by the LB and RB T-DNA sequences, as described above. The plasmid backbone also contains a bacterial selection marker cassette that encodes the KanR gene, and which is in reverse orientation on the plasmid vector 665. The Cannabaceae plant parts (e.g., Cannabis) were transformed using the R. rhizogenes strain A4 (ATCC43057). The plasmid vector 665 comprises SEQ ID NO: 54. Further identified is the sequence of the betalain cassette (SEQ ID NO: 55), the plant selectable marker cassette (SEQ ID NO: 30), the T-DNA borders of the RB (SEQ ID NO: 35) and LB (SEQ ID NO: 36), the LacZ cassette (SEQ ID NO: 37) and the bacterial selection marker cassette (SEQ ID NO: 40). The betalain cassette (SEQ ID NO: 54) encodes the FMV promoter (SEQ ID NO: 23), CYP76AD6 (SEQ ID NO: 56), P2A 1 (SEQ ID NO: 45), DODA with stop (SEQ ID NO: 57), and a rbcS terminator (SEQ ID NO: 29). The plant selectable marker cassette (SEQ ID NO: 30) encodes a 35S promoter (SEQ ID NO: 31), an ST LS1 nptll intron (SEQ ID NO: 32), a nptll exon (SEQ ID NO: 33), and a 35S terminator (SEQ ID NO: 34). The LacZ cassette (SEQ ID NO: 37) encodes the LacZ promoter (SEQ ID NO: 38) and LacZ gene (SEQ ID NO: 39). The bacterial selection marker cassette (SEQ ID NO: 40) encodes the KanR promoter (SEQ ID NO: 41) and KanR gene (SEQ ID NO: 42).

FIG. 6H illustrates an example plasmid vector 671 that includes a DNA sequence coding for a plurality of enzymes to produce betalain (e.g., betanidin and betaxanthin). The plasmid vector 671 contains the gene encoding the enzymes CYP76AD1 and DODA driven by a FMV promoter, with CYP76AD1 linked to DODA by a 2A self-cleaving peptide P2A. The plasmid vector 671 further includes a plant selectable marker cassette and a LacZ cassette, which are in reverse orientation on the plasmid vector 671 and as described by plasmid vector 652, the features of which are not repeated. The gene cassettes are flanked by the LB and RB T-DNA sequences, as described above. The plasmid backbone also contains a bacterial selection marker cassette that encodes the KanR gene, and which is in reverse orientation on the plasmid vector 671. The Cannabaceae plant parts (e.g., Cannabis) were transformed using the R. rhizogenes strain A4 (ATCC43057). The plasmid vector 671 sequence comprises SEQ ID NO: 58. Further identified is the sequence of the betalain cassette (SEQ ID NO: 59), the plant selectable marker cassette (SEQ ID NO: 30), the T-DNA borders of the RB (SEQ ID NO: 35) and LB (SEQ ID NO: 36), the LacZ cassette (SEQ ID NO: 37) and the bacterial selection marker cassette (SEQ ID NO: 40). The betalain cassette (SEQ ID NO: 59) encodes the FMV promoter (SEQ ID NO: 23), CYP76AD1 (SEQ ID NO: 24), P2A 1 (SEQ ID NO: 45), DODA with stop (SEQ ID NO: 57), and a rbcS terminator (SEQ ID NO: 29). The plant selectable marker cassette (SEQ ID NO: 30) encodes a 35S promoter (SEQ ID NO: 31), an ST LS1 nptll intron (SEQ ID NO: 32), a nptll exon (SEQ ID NO: 33), and a 35S terminator (SEQ ID NO: 34). The LacZ cassette (SEQ ID NO: 37) encodes the LacZ promoter (SEQ ID NO: 38) and LacZ gene (SEQ ID NO: 39). The bacterial selection marker cassette (SEQ ID NO: 40) encodes the KanR promoter (SEQ ID NO: 41) and KanR gene (SEQ ID NO: 42).

FIG. 61 illustrates an example plasmid vector 672 that includes a DNA sequence coding for a plurality of enzymes to produce betalain (e.g., betanidin). The plasmid vector 672 contains the gene encoding the enzymes CYP76AD1, DODA, and glucosyltransferase driven by a FMV promoter, with CYP76AD1 linked to DODA and DODA linked to glucosyltransferase by 2A self-cleaving peptides P2A. The plasmid vector 672 further includes a plant selectable marker cassette and a LacZ cassette, which are in reverse orientation on the plasmid vector 672 and as described by plasmid vector 652, the features of which are not repeated. The gene cassettes are flanked by the LB and RB T-DNA sequences, as described above. The plasmid backbone also contains a bacterial selection marker cassette that encodes the KanR gene, and which is in reverse orientation on the plasmid vector 672. The Cannabaceae plant parts (e.g., Cannabis) were transformed using the R. rhizogenes strain A4 (ATCC43057). The plasmid vector 672 sequence comprises SEQ ID NO: 60. Further identified is the sequence of the betalain cassette (SEQ ID NO: 44), the plant selectable marker cassette (SEQ ID NO: 61), the T-DNA borders of the RB (SEQ ID NO: 35) and LB (SEQ ID NO: 36), the LacZ cassette (SEQ ID NO: 37) and the bacterial selection marker cassette (SEQ ID NO: 40). The betalain cassette (SEQ ID NO: 44) encodes the FMV promoter (SEQ ID NO: 23), CYP76AD1 (SEQ ID NO: 24), P2A 1 (SEQ ID NO: 45), DODA (SEQ ID NO: 26), P2A 2 (SEQ ID NO: 46), glucosyltransferase (SEQ ID NO: 28), and a rbcS terminator (SEQ ID NO: 29). The plant selectable marker cassette (SEQ ID NO: 61) encodes a VuUbi promoter (SEQ ID NO: 62), chloroplast transit peptide (SEQ ID NO: 63), an SpcN (e.g., Spec) (SEQ ID NO: 64), and a NOS terminator (SEQ ID NO: 65). The LacZ cassette (SEQ ID NO: 37) encodes the LacZ promoter (SEQ ID NO: 38) and LacZ gene (SEQ ID NO: 39). The bacterial selection marker cassette (SEQ ID NO: 40) encodes the KanR promoter (SEQ ID NO: 41) and KanR gene (SEQ ID NO: 42).

FIG. 6J illustrates an example plasmid vector 674 that includes a DNA sequence coding for transforming adult plant parts, such as petioles, internodes, and/or leafs from hop and/or Cannabis plants. The plasmid vector 674 contains the gene encoding YFP driven by the FMV promoter along with the SPCN gene which confers resistance to spectinomycin in the transformed plant cells driven by the Ubi3 promoter. These two gene cassettes are flanked by the LB and RB T-DNA sequences allowing for transfer of the entire sequence or transgene into the plant cells by the bacterium strain of R. rhizogenes. The plasmid backbone also contains a KanR gene which allows for selection and maintenance of the plasmid within the R. rhizogenes strain. The Cannabaceae plant parts (e.g., hops and Cannabis) were transformed using the R. rhizogenes strain A4 (ATCC43057). The plasmid vector 674 sequence comprises SEQ ID NO: 66. Further identified is the sequence of the YFP cassette (SEQ ID NO: 67), the plant selectable marker cassette (SEQ ID NO: 71), the T-DNA borders of the RB (SEQ ID NO: 76) and LB (SEQ ID NO: 77), and the bacterial selection marker cassette (SEQ ID NO: 78). The YFP cassette (SEQ ID NO: 67) encodes the FMV promoter (SEQ ID NO: 68), YFP (SEQ ID NO: 69), and a rbcS terminator (SEQ ID NO: 70). The plant selectable marker cassette (SEQ ID NO: 71) encodes a VuUbi promoter (SEQ ID NO: 72), chloroplast transit peptide (SEQ ID NO: 73), an SpcN (e.g., Spec) (SEQ ID NO: 74), and a NOS terminator (SEQ ID NO: 75). The bacterial selection marker cassette (SEQ ID NO: 78) encodes the KanR promoter (SEQ ID NO: 79) and KanR gene (SEQ ID NO: 80).

FIG. 6K illustrates an example plasmid vector 675 that includes a DNA sequence coding for transcription factors to produce an anthocyanin. The plasmid vector 675 contains the gene encoding the transcription factors MYB (e.g., LAP1) driven by a Ubi3 promoter. The plasmid vector 675 further includes a plant selectable marker cassette and a bacterial selection makers cassette, which is in reverse orientation on the plasmid vector 675 and as described by plasmid vector 652. The gene cassettes are flanked by the LB and RB T-DNA sequences, as described above. The Cannabaceae plant parts (e.g., Cannabis) were transformed using the R. rhizogenes strain A4 (ATCC43057). The plasmid vector 675 sequence comprises SEQ ID NO: 81. Further identified is the sequence of the LAP1 cassette (SEQ ID NO: 82), the plant selectable marker cassette (SEQ ID NO: 86), the T-DNA borders of the RB (SEQ ID NO: 91) and LB (SEQ ID NO: 92), and the bacterial selection marker cassette (SEQ ID NO: 93). The LAP1 cassette (SEQ ID NO: 82) encodes the VaUbi3 promoter (SEQ ID NO: 83), LAP1 (SEQ ID NO: 84), and the NOS terminator (SEQ ID NO: 85). The plant selectable marker cassette (SEQ ID NO: 86) encodes a MtEF1A promoter (SEQ ID NO: 87), ST LS1 nptll intron (SEQ ID NO: 88), nptll exon (SEQ ID NO: 89), and a 35s terminator (SEQ ID NO: 90). The bacterial selection marker cassette (SEQ ID NO: 93) encodes the KanR promoter (SEQ ID NO: 94) and KanR gene (SEQ ID NO: 95).

Consistent with the above description, bacterium strains and plant source material were prepared as follows. Five to seven days prior to the experiment, the desired R. rhizogenes strain was streaked out onto an AB minimal media agar plate (see media recipes) with appropriate antibiotics. The plates were incubated at 28 degrees C. until the day of the experiment. Six (6) days prior to the experiment, 50-100 Cannabis seeds were surface sterilized with 10 mL concentrated sulfuric acid for 10-30 seconds, and then is removed from the sulfuric acid and rinsed with sterile water, such as washed twice with sterile water. The seeds were soaked in 30% hydrogen peroxide (H₂O₂) for 20 minutes and washed twice with sterile water. The seeds were allowed to imbibe in sterile water overnight (e.g., for 16-24 hours) with some gentle agitation, either in a conical tube placed in a motorized invertor or a sealed petri dish on a rotary shaker. Using forceps and a stereomicroscope, the seed coats and endosperm were removed before plating the embryos onto 8P-MS-G media plates (see media recipes) with a maximum of five embryos per plate. The plates were sealed with parafilm and placed in the dark for three 3 days. The plates were transferred to a 16/8-hour light/dark incubator (75 lumens, 23 degrees C.) for two additional days.

Infection of Cannabis hypocotyl tissue for PCM production was performed as follows. Five hours prior to infection, a loopful of bacteria from the plates was suspended in 1 mL of sterile water containing 100 μM acetosyringone. The bacterial suspension was maintained in a dark lab drawer at room temperature. The Cannabis seedlings were removed from the incubator and the following steps were performed.

To infect whole Cannabis seedlings, the point of a scalpel was used to make a small wound in the hypocotyl of each seedling, approximately 5-10 mm above the top of the radicle. Immediately after wounding, the wound was inoculated with 20 μL of bacterial suspension. After a minimum of 10 minutes, the seedlings were transferred to PCM co-cultivation media (a Murashige and Skoog (MS) media plate, see media recipes). The number of seedlings per plate was limited to five inoculated seedlings. The plates were sealed with parafilm and placed in the dark overnight (22 degrees C. to 23 degrees C.). The plates were removed from the dark chamber and transferred to a 16/8-hour light/dark incubator (75 lumens, 23 degrees C.) for one additional day for a total of two days of co-cultivation.

For whole Cannabis seedlings, each seedling was transferred to an individual PCM media plate (e.g., an MS media plate, see media recipes) containing 500 μg/mL of cefotaxime (PCM+Cef500), and care was exercised to ensure that the previously wounded part of the hypocotyl was touching the medium. The plates, such Phytatrays™, were closed and placed in a light chamber for two weeks.

To infect hypocotyl tissue, the point of a scalpel was used to cut off the radicle and cotyledon of each seedling. The hypocotyl were then cut into 5-10 mm segments. The cut segments were gathered into piles and immediately inoculated with 50-100 μl of bacterial suspension. Care was taken to ensure that all segments, particularly the cut ends, were covered with a layer of bacterium strain. After a minimum of 10 minutes, the segments were transferred to PCM co-cultivation media (MS media, see media recipes) keeping the segments in a pile. The number of piles of segment per plate was limited to four piles of segments. The plates were sealed with parafilm and placed in the dark overnight (22 degrees C. to 23 degrees C.). The plates were removed from the dark chamber and transferred to a 16/8-hour light/dark incubator (75 lumens, 23 degrees C.) for one additional day for a total of two days of co-cultivation.

For hypocotyl tissue, each pile was transferred to an individual PCM media plate (an MS media plate, see media recipes) containing 500 μg/mL of cefotaxime (PCM+Cef500), and care was exercised to ensure that the segments were spread out evenly over the surface of the plates. The plates, such Phytatrays™, were closed and placed in a light chamber for two weeks.

To isolate and maintain the PCM clones, after two weeks, the tissue was transferred to fresh PCM media plate (see media recipes) containing 500 μg/mL of cefotaxime (PCM+Cef500) before returning the plates to the light chamber for two more weeks. After another two weeks, the tissue was screened for root formation. Any developing roots were removed using a scalpel and transferred to another PCM media plate (PCM+Cef500) for sub-culturing. These plates were sealed with parafilm and placed in the dark (22 degrees C. or 23 degrees C.). When selecting for the presence of a secondary T-DNA (a fluorescent protein or TALEN expressing cassette) insertion originating from a binary vector, an appropriate selection compound is added to the above media at the root sub-culturing stage. In the particular experiment including YFP, spectinomycin was added. This allows for the growth of only PCM clones containing and expressing both the binary vector and PCM T-DNAs.

Any whole seedlings or hypocotyl segments which have not developed roots were returned to the light chamber after transferring to fresh media for another two weeks. If the tissue has not developed roots after three rounds of media transfers, for a total of 8 weeks, it was discarded. For a particularly successful transformation, root tissue was excised from the same original explant multiple times. The original explant will usually start to die after 8 weeks of culture.

Plates of sub-cultured roots were transferred to fresh PCM media every two to three weeks with the concentration of cefotaxime in the medium being gradually reduced from 500 μg/mL (for two rounds of transfers) to 300 μg/mL (for one to two rounds of transfers) to 100 μg/mL (for one to two rounds of transfers). Other selection agents should be maintained at the same concentration throughout. Healthy PCM clones grow to be quite large (will cover the surface of the plate) so a portion of each clone was transferred to fresh media (between 1-2 square cm) while the remaining tissue is discarded. In some experimental embodiments, only one clone is maintained per plate.

After the final round of sub-culturing on the PCM media containing 100 μg/mL cefotaxime (PCM+Cef100), the clones were transferred to medium containing no antibiotics (same as PCM co-cultivation media media) because all R. rhizogenes should be eliminated from the tissue. The clones can be maintained indefinitely on this media type and will only need to be transferred to fresh media every two to three weeks.

To grow PCM tissue in liquid media, a piece of root tissue approximately one cm² is placed in a 250 mL flask containing 50 mL of liquid medium. This should be done with root tissue that has already had any R. rhizogenes eliminated resulting in a “clean” clone. Sterility should be maintained at all times. The flask was placed on a shaker in the dark with a speed of 100 rotations per minute (rpm) and allowed to grow for 7 days before removing the spent media with a sterile pipette and adding 50 mL of fresh media. After two to three weeks, the tissue should double in size. If no bacterial or fungal contamination is present, a larger flask was used, for example a 500 mL flask containing 250 mL of MS liquid media or a 6 L flask containing 3 L of media, to bulk up the root tissue. In other experiments, DKW liquid media was used. The tissue was cultured and maintained in the same manner as described above. In embodiments, the plant part was cultured in temperatures of between about 22 degrees C. and about 25 degrees C. and/or in different light and dark conditions for growth conditions. The PCM grew in a variety of light and dark conditions.

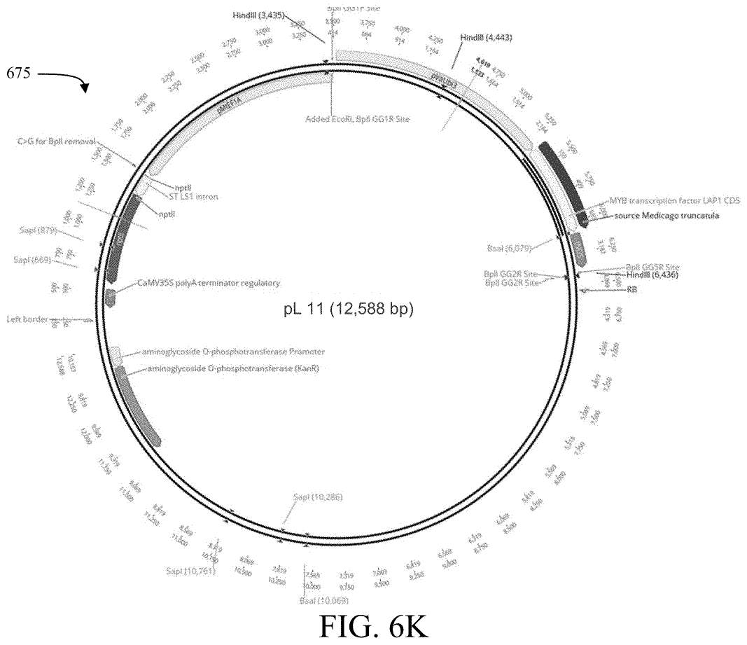

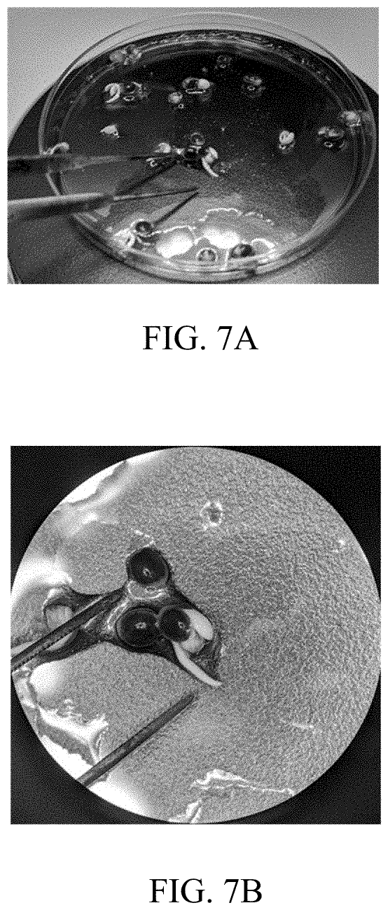

FIGS. 7A-7B illustrate example seedling preparations, consistent with the present disclosure. As illustrated in FIGS. 7A-7B, PCM co-cultivated Cannabis seedlings demonstrated growth following the procedure described above.

R. rhizogenes was prepared as follows for infecting and transforming plant parts. 25 mL of cells of the desired R. rhizogenes strain were grown overnight in YEP media with appropriate antibiotics. The overnight culture was inoculated with a single colony from a fresh AB solid media plate (see media recipes) and with the appropriate antibiotics. With the protocol, the cells were kept on ice. Cells were collected from the 25 mL culture by centrifugation of the cells into a sterile conical tube with shaking at 4 degrees C. at 6000 rpm for ten minutes. The cells were washed three times with 5 mL of ice-cold sterile water, with the tube in an ice bucket with a mixture of ice to ensure a low temperature. Care was taken to ensure that the outside surface of the tube was clean to prevent contamination of the cells. The cells were then washed one time with 5 mL of ice-cold 10% glycerol. 800 μl of the 10% glycerol was used to suspend cells, resulting in approximately 1000 μl of cell suspension. The competent cells were aliquoted into two microfuge tubes with 60 μl in each tube. Electroporation was then performed or the tubes were stored in −80 degrees C. freezer for later electroporation. In some embodiments, electroporation can be implemented using at least some of the features described in Chassy, et al., “Transformation of Bacteria by Electroporation”, Trends Biotechnol, Vol. 6, Issue 12, 303-309, 1988, which is herein incorporated in its entirety for its teaching. In some experiments, after electroporation, 1 mL of YEP medium was used to resuspend the cells. The cells were then transferred into a sterile test tube and incubated at 28-30 degrees C. with shaking for two hours. The cells were transferred to a microfuge tube, and a series of 10-fold dilutions with 0.9% sterile NaCl or YEP liquid media were made. 100 μl of the undiluted culture was plated and each dilution (e.g. 10⁻¹, 10⁻²) onto separate AB sucrose media plates with appropriate antibiotics. The original tube was kept at 4 degrees C. Pinprick colonies should appear within 48 hours. The number of colonies were counted three days after transformation and this number can be used to determine the competency of the cells. Care was taken to obtain single colonies (not confluent lawn) from the selection plates before proceeding with the experiments.

In particular experiments, multiple bacterium strains were assessed to identify transformation frequencies. R. rhizogenes strains of K599, A4 (ATCC43057), R1000 (ATCC43056), and TR104 (ATCC13333) were used to infect Cannabis whole seedlings and/or hypocotyl segments. Transformation frequency was determined by the number of plants or segments which exhibited PCM formation out of the total assayed over multiple experiments. A4 gave a transformation frequency of 68% to 89%, TR104 a frequency of 28% to 67%, K599 a frequency of around 2%, and R1000 a frequency of less than 2%. PCM clones isolated from tissues infected with A4 also had the best growth in tissue culture and have been able to be maintained indefinitely. TR104 derived clones eventually lose vitality after two or three times of being sub-cultured. In various experiments, A4 was used for transformation experiments.

To create the 8P-MS-G media (Phytatrays™ or plates), the following protocol and volumes were used to make a 1L solution of media:
800 mL ddH2O; 10 g Sucrose; 4.43 g MS Basal Salts+Vitamins (Phytotech, M519); the solution was brought to volume with 1000 mL ddH2O; the pH was adjusted to 5.7 with titration of KOH; and 3.58 g Gelzan™ (Phytotech, G3251). The media was autoclaved on the liquid cycle for 25 minutes and cooled to 55 degrees C. and poured 100 mL per Phytatray™ or 25 mL or 50 mL per 100×25 mm plates.

To create the LB media (culture tubes), the following protocol and volumes were used to make a 1L solution of media:
800 mL of ddH₂O; 25 g of LB (Sigma: L3522); and the solution was brought to volume with 1000 mL of ddH₂O.
   The media was autoclaved on liquid cycle for 25 minutes.

To create the LB agar media (plates), the following protocol and volumes were used to make a 1L solution of media:
800 mL of ddH₂O; 25 g of LB (Sigma: L3522); 15 g of Agar (Sigma: A5306); and the solution was brought to volume with 1000 ml of ddH₂O.
   The media was autoclaved on liquid cycle for 25 minutes and cooled to 55 degrees C. and poured into 25 mL into 100×15 mm plates.

To create AB Salts (20X), the following protocol and volumes were used:
700 mL of ddH₂O; 20 g of NH₄Cl; 6 g of MgSO₄*7H₂O; 3 g of KCl;
0.2 g of CaCl₂; 50 mg of FeSO₄*7H₂O; the pH was adjusted to 7.0 with KOH; and the solution was brought to volume with 1000 mL of ddH₂O.

To create AB Buffer (20×), the following protocol and volumes were used to form 1L of the media:
700 mL of ddH₂O; 60 g of K₂HPO₄; 20 g of NaH₂PO₄; and the solution was brought to volume with 1000 mL of ddH₂O.

To create AB minimal agar media (liquid), the following protocol and volumes were used to form 1L of media:

700 mL of ddH₂O; 5 g of Sucrose; the solution was brought to volume with 1000 mL of ddH₂O; 50 mL of 20× AB Salts; and 50 mL of 20× AB Buffer. The media was autoclaved on liquid cycle for 25 minutes.

To create AB minimal media (plates), the following protocol and volumes were used to form 1L of media:
700 mL of ddH₂O; 5 g of Sucrose; the solution was brought to volume with 1000 mL of ddH₂O; 50 mL of 20× AB Salts; 50 mL of 20× AB Buffer; and 15 g of Agar (Sigma: A5306).
   The media was autoclaved on liquid cycle for 25 minutes and cooled to 55 degrees C. and poured into 100×15 mm plates.

To create YEP media (liquid), the following protocol and volumes were used to form 1L of media:

800 mL of ddH₂O; 10 g of Bacto-peptone; 5 g of Yeast extract; 5 g of NaCl; and the solution was brought to volume with 1000 mL of ddH₂O.

The media was filter sterilized.

To create YEP media (plates), the following protocol and volumes were used to form 1L of media:

800 mL of ddH₂O; 10 g of Bacto-peptone; 5 g of Yeast extract; 5 g of NaCl; the solution was brought to volume with 1000 mL of ddH₂O; and 15 g of Agar (Sigma: A5306).

The media was autoclaved on liquid cycle for 25 minutes and cooled to 55 degrees C. and poured into 100×15 mm plates.

To create PCM media (plates), which can be referred to as an MS media with antibiotics or MS liquid, the following protocol and volumes were used:
800 mL of ddH₂O; 30 g of Sucrose (Phytotech: S9378); 4.43 g of MS basal salts+Vitamins (Phytotech: M519); the solution was brought to volume with 1000 mL of ddH₂O; the pH was adjusted to 5.8 by titration of KOH; and 6 g of Agarose (Phytotech: A6013).
   The media was autoclaved on liquid cycle for 25 minutes and cooled to 55 degrees C. before adding 500 mg/L, 300 mg/L, or 100 mg/L of cefotaxime and pouring 50 mL per 100×25 mm plates. In some experimental embodiments, DKW basal salts were used in place of MS basal salts.

To create PCM co-cultivation media (plates), which can be referred to as an MS media without antibiotics, the following protocol and volumes were used to create 1L of media:
800 mL of ddH₂O; 30 g of Sucrose (Phytotech: S9378); 4.43 g of MS basal salts+Vitamins (Phytotech: M519); the solution was brought to volume with 1000 mL of ddH₂O; the pH was adjusted to 5.8 by titration of KOH; and 6 g of Agarose (Phytotech: A6013).
   The media was autoclaved on liquid cycle for 25 minutes and cooled to 55 degrees C. and poured 50 mL per 100×25 mm plate. In some experimental embodiments, DKW basal salts were used in place of MS basal salts.

As noted above and further described below, various different types of culture mediums were used to enhance growth of PCM tissue using transformed plant parts. Various experimental embodiments were directed to use and assessment of different culture mediums. The following provides different example culture media used and protocols creating the same.

To create a liquid culture media containing DKW and B5, referred to as PCM DKW-B5 liquid, the following protocol and volumes were used to create 1L of media:
800 mL of ddH₂O; 30 g of Sucrose (S9378); 5.22 g of DKW basal salt mixture (D190); 1 mL of Gamborg Vitamin solution [1000×] (G219); the solution was brought to volume with 180.132 mL of ddH₂O; the pH was adjusted to 5.8 with KOH.
   The media was autoclaved on AGAR cycle with MediaClave.

To create a culture media containing DKW and MES, referred to as PCM 5 gL MES, the following protocol and volumes were used to create 1L of media:
800 mL of ddH₂O; 30 g of Sucrose (S9378); 5.22 g of DKW basal salt mixture (D190); 1 mL of Gamborg Vitamin solution [1000×] (G219); 5 g MES (M825); the solution was brought to volume with 180.132 mL of ddH₂O; the pH was adjusted to 5.8 with KOH.
   The media was autoclaved on AGAR cycle with MediaClave.

To create a liquid culture media containing DKW and MES, referred to as PCM DKW-MES liquid, the following protocol and volumes were used to create 1L of media:
800 mL of ddH₂O; 30 g of Sucrose (S9378); 5.22 g of DKW basal salt mixture (D190); 1 mL of Gamborg Vitamin solution [1000×] (G219); 1 g MES (M825); the solution was brought to volume with 180.132 mL of ddH₂O; the pH was adjusted to 5.8 with KOH.
   The media was autoclaved on AGAR cycle with MediaClave. In some experimental embodiments, it was identified that culture media with 1 g MES (e.g., the DKW-MES liquid) performed better than 5 g MES, such as PCM 5 gL MES.

To create another culture media containing DKW and MES (solid media), referred to as PCM MES, the following protocol and volumes were used to create 1L of media:
800 mL of ddH₂O; 30 g of Sucrose (S9378); 5.22 g of DKW basal salt mixture (D190); 1 mL of Gamborg Vitamin solution [1000×] (G219); 1 g MES (M825); the solution was brought to volume with 180.132 mL of ddH₂O; the pH was adjusted to 5.8 with KOH; 6 g Agarose (A6013) was added.
   The media was autoclaved on AGAR cycle with MediaClave.

To create a culture media containing DKW, MES, and Cefotaxime, referred to as PCM+Cef300+MES, the following protocol and volumes were used to create 1L of media:
800 mL of ddH₂O; 30 g of Sucrose (S9378); 5.22 g of DKW basal salt mixture (D190); 1 mL of Gamborg Vitamin solution [1000×] (G219); 1 g MES (M825); the solution was brought to volume with 180.132 mL of ddH₂O; the pH was adjusted to 5.8 with KOH; 6 g Agarose (A6013) was added.
   The media was autoclaved on AGAR cycle with MediaClave, and post autoclave 300 mg of Cefotaxime [250 mg/L] was added.

To create a liquid culture media containing DKW and B5, referred to as PCM DKW-B5-15 g/L liquid, the following protocol and volumes were used to create 1L of media:
800 mL of ddH₂O; 15 g of Sucrose (S9378); 5.22 g of DKW basal salt mixture (D190); 1 mL of Gamborg Vitamin solution [1000×] (G219); the solution was brought to volume with 189.566 mL of ddH₂O; the pH was adjusted to 5.8 with KOH.
   The media was autoclaved on AGAR cycle with MediaClave.

To create a liquid culture media containing DKW and B5, referred to as PCM DKW-B5-45g/L liquid, the following protocol and volumes were used to create 1L of media:
800 mL of ddH₂O; 45 g of Sucrose (S9378); 5.22 g of DKW basal salt mixture (D190); 1 mL of Gamborg Vitamin solution [1000×] (G219); the solution was brought to volume with 170.698 mL of ddH₂O; the pH was adjusted to 5.8 with KOH.
   The media was autoclaved on AGAR cycle with MediaClave.

To create a liquid culture media containing DKW and B5, referred to as PCM DKW-B5-5g/L liquid, the following protocol and volumes were used to create 1L of media:

800 mL of ddH₂O; 5 g of Sucrose (S9378); 5.22 g of DKW basal salt mixture (D190); 1 mL of Gamborg Vitamin solution [1000×] (G219); the solution was brought to volume with 195.855 mL of ddH₂O; the pH was adjusted to 5.8 with KOH.
The media was autoclaved on AGAR cycle with MediaClave.

To create a liquid culture media containing DKW and B5, referred to as PCM DKW-B5-60 g/L liquid, the following protocol and volumes were used to create 1L of media:
800 mL of ddH₂O; 60 g of Sucrose (S9378); 5.22 g of DKW basal salt mixture (D190); 1 mL of Gamborg Vitamin solution [1000×] (G219); the solution was brought to volume with 161.264 mL of ddH₂O; the pH was adjusted to 5.8 with KOH.
   The media was autoclaved on AGAR cycle with MediaClave.

To create a liquid culture media containing DKW and B5, referred to as PCM DKW-B5-15 g/L liquid, the following protocol and volumes were used to create 1L of media:

800 mL of ddH₂O; 15 g of Sucrose (S9378); 5.22 g of DKW basal salt mixture (D190); 1 mL of Gamborg Vitamin solution [1000×] (G219); the solution was brought to volume with 189.566 mL of ddH₂O; the pH was adjusted to 5.8 with KOH.

The media was autoclaved on AGAR cycle with MediaClave. In some experiments, the liquid culture media with different sucrose concentrations, as listed above, were assessed and were not selected for optimized growth conditions. However, embodiments are not so limited.

To create a liquid culture media containing DKW and B5, referred to as 0.25× DKW-B5 liquid, the following protocol and volumes were used to create 1L of media:
800 mL of ddH₂O; 30 g of Sucrose (S9378); 1.31 g of DKW basal salt mixture (D190); 1 mL of Gamborg Vitamin solution [1000×] (G219); the solution was brought to volume with 180.132 mL of ddH₂O; the pH was adjusted to 5.8 with KOH.
   The media was autoclaved on AGAR cycle with MediaClave.

To create a liquid culture media containing DKW and B5, referred to as 0.5× DKW-B5 liquid, the following protocol and volumes were used to create 1L of media:
800 mL of ddH₂O; 30 g of Sucrose (S9378); 2.61 g of DKW basal salt mixture (D190); 1 mL of Gamborg Vitamin solution [1000×] (G219); the solution was brought to volume with 180.132 mL of ddH₂O; the pH was adjusted to 5.8 with KOH.
   The media was autoclaved on AGAR cycle with MediaClave.

To create a liquid culture media containing DKW and B5, referred to as 0.75× DKW-B5 liquid, the following protocol and volumes were used to create 1L of media:
800 mL of ddH₂O; 30 g of Sucrose (S9378); 3.92 g of DKW basal salt mixture (D190); 1 mL of Gamborg Vitamin solution [1000×] (G219); the solution was brought to volume with 180.132 mL of ddH₂O; the pH was adjusted to 5.8 with KOH.
   The media was autoclaved on AGAR cycle with MediaClave.

To create a liquid culture media containing DKW and B5, referred to as 1.5× DKW-B5 liquid, the following protocol and volumes were used to create 1L of media:
800 mL of ddH₂O; 30 g of Sucrose (S9378); 7.83 g of DKW basal salt mixture (D190); 1 mL of Gamborg Vitamin solution [1000×] (G219); the solution was brought to volume with 180.132 mL of ddH₂O; the pH was adjusted to 5.8 with KOH.
   The media was autoclaved on AGAR cycle with MediaClave. In some experiments, the liquid culture media with different DKW concentrations, as listed above, were assessed and were not selected for optimized growth conditions. However, embodiments are not so limited.

To create a culture media containing DKW, B5 and Cefotaxime, referred to as DKW-B5+Cf100, the following protocol and volumes were used to create 1L of media:
800 mL of ddH₂O; 30 g of Sucrose (S9378); 5.22 g of DKW basal salt mixture (D190); 1 mL of Gamborg Vitamin solution [1000×] (G219); the solution was brought to volume with 179.732 mL of ddH₂O; the pH was adjusted to 5.8 with KOH; 6 g Agarose (A6013) was added.
   The media was autoclaved on AGAR cycle with MediaClave, and post autoclave 100 mg of Cefotaxime [250 mg/L] was added.

To create another culture media containing DKW, B5 and Cefotaxime, referred to as DKW-B5+Cf300, the following protocol and volumes were used to create 1L of media:
800 mL of ddH₂O; 30 g of Sucrose (S9378); 5.22 g of DKW basal salt mixture (D190); 1 mL of Gamborg Vitamin solution [1000×] (G219); the solution was brought to volume with 178.932 mL of ddH₂O; the pH was adjusted to 5.8 with KOH; 6 g Agarose (A6013) was added.
   The media was autoclaved on AGAR cycle with MediaClave, and post autoclave 300 mg of Cefotaxime [250 mg/L] was added.

To create another culture media containing DKW, B5 and Cefotaxime, referred to as DKW-B5+Cf500, the following protocol and volumes were used to create 1L of media:
800 mL of ddH₂O; 30 g of Sucrose (S9378); 5.22 g of DKW basal salt mixture (D190); 1 mL of Gamborg Vitamin solution [1000×] (G219); the solution was brought to volume with 178.132 mL of ddH₂O; the pH was adjusted to 5.8 with KOH; 6 g Agarose (A6013) was added.
   The media was autoclaved on AGAR cycle with MediaClave, and post autoclave 500 mg of Cefotaxime [250 mg/L] was added.

To create a culture media containing DKW, B5, Cefotaxime, and Spectinomycin, referred to as DKW-B5+Cf500+Spec10, the following protocol and volumes were used to create 1L of media:
800 mL of ddH₂O; 30 g of Sucrose (S9378); 5.22 g of DKW basal salt mixture (D190); 1 mL of Gamborg Vitamin solution [1000×] (G219); the solution was brought to volume with 178.932 mL of ddH₂O; the pH was adjusted to 5.8 with KOH; 6 g Agarose (A6013) was added.
   The media was autoclaved on AGAR cycle with MediaClave, and post autoclave 500 mg of Cefotaxime [250 mg/L] and 10 mg Spectinomycin [50 mg/mL] (S4014) was added.

To create another culture media containing DKW, B5, Cefotaxime, and Spectinomycin, referred to as DKW-B5+Cf500+Spec20, the following protocol and volumes were used to create 1L of media:
800 mL of ddH₂O; 30 g of Sucrose (S9378); 5.22 g of DKW basal salt mixture (D190); 1 mL of Gamborg Vitamin solution [1000×] (G219); the solution was brought to volume with 178.932 mL of ddH₂O; the pH was adjusted to 5.8 with KOH; 6 g Agarose (A6013) was added.
   The media was autoclaved on AGAR cycle with MediaClave, and post autoclave 500 mg of Cefotaxime [250 mg/L] and 20 mg Spectinomycin [50 mg/mL] (S4014) was added.

To create another culture media containing DKW, B5, Cefotaxime, and Spectinomycin, referred to as DKW-B5+Cf500+Spec30, the following protocol and volumes were used to create 1L of media:

800 mL of ddH₂O; 30 g of Sucrose (S9378); 5.22 g of DKW basal salt mixture (D190); 1 mL of Gamborg Vitamin solution [1000×] (G219); the solution was brought to volume with 178.932 mL of ddH₂O; the pH was adjusted to 5.8 with KOH; 6 g Agarose (A6013) was added.

The media was autoclaved on AGAR cycle with MediaClave, and post autoclave 500 mg of Cefotaxime [250 mg/L] and 30 mg Spectinomycin [50 mg/mL] (S4014) was added.

To create another culture media containing DKW, B5, Cefotaxime, and Spectinomycin, referred to as DKW-B5+Cf500+Spec40, the following protocol and volumes were used to create 1L of media:
800 mL of ddH₂O; 30 g of Sucrose (S9378); 5.22 g of DKW basal salt mixture (D190); 1 mL of Gamborg Vitamin solution [1000×] (G219); the solution was brought to volume with 178.932 mL of ddH₂O; the pH was adjusted to 5.8 with KOH; 6 g Agarose (A6013) was added.
   The media was autoclaved on AGAR cycle with MediaClave, and post autoclave 500 mg of Cefotaxime [250 mg/L] and 40 mg Spectinomycin [50 mg/mL] (S4014) was added. In some experiments, the above (and below) example media were used to assess different Spectinomycin concentrations.

To create another culture media containing DKW, B5, Cefotaxime, and G419 Sulfate, referred to as DKW-B5+Cf500+G418, the following protocol and volumes were used to create 1L of media:
800 mL of ddH₂; 30 g of Sucrose (S9378); 5.22 g of DKW basal salt mixture (D190); 1 mL of Gamborg Vitamin solution [1000×] (G219); the solution was brought to volume with 178.132 mL of ddH₂; the pH was adjusted to 5.8 with KOH; 6 g Agarose (A6013) was added.
   The media was autoclaved on AGAR cycle with MediaClave, and post autoclave 500 mg of Cefotaxime [250 mg/L] and 5 mg G419 Sulfate [50 mg/mL] was added.

To create another culture media containing DKW, B5, Cefotaxime, and Spectinomycin, referred to as DKW-B5+Cf300+Spec100, the following protocol and volumes were used to create 1L of media:
800 mL of ddH₂O; 30 g of Sucrose (S9378); 5.22 g of DKW basal salt mixture (D190); 1 mL of Gamborg Vitamin solution [1000×] (G219); the solution was brought to volume with 178.932 mL of ddH₂O; the pH was adjusted to 5.8 with KOH; 6 g Agarose (A6013) was added.
   The media was autoclaved on AGAR cycle with MediaClave, and post autoclave 300 mg of Cefotaxime [250 mg/L] and 100 mg Spectinomycin [50 mg/mL] (S4014) was added.

To create another culture media containing DKW, B5, Cefotaxime, and Spectinomycin, referred to as DKW-B5+Cf500+Spec100, the following protocol and volumes were used to create 1L of media:
800 mL of ddH₂O; 30 g of Sucrose (S9378); 5.22 g of DKW basal salt mixture (D190); 1 mL of Gamborg Vitamin solution [1000×] (G219); the solution was brought to volume with 178.132 mL of ddH₂O; the pH was adjusted to 5.8 with KOH; 6 g Agarose (A6013) was added.
   The media was autoclaved on AGAR cycle with MediaClave, and post autoclave 500 mg of Cefotaxime [250 mg/L] and 100 mg Spectinomycin [50 mg/mL] (S4014) was added.

To create a culture media containing DKW and Cefotaxime, referred to as DKW+Cef300, the following protocol and volumes were used to create 1L of media:
800 mL of ddH₂O; 30 g of Sucrose (S9378); 5.22 g of DKW basal salt mixture (D190); the solution was brought to volume with 179.932 mL of ddH₂O; the pH was adjusted to 5.8 with KOH; 6 g Agarose (A6013) was added.
   The media was autoclaved on AGAR cycle with MediaClave, and post autoclave 300 mg of Cefotaxime [250 mg/L] was added.

To create a culture media containing WPM and B5, referred to as WPM-B5−30, the following protocol and volumes were used to create 1L of media:
800 mL of ddH₂O; 30 g of Sucrose (S9378); 2.3 g of WPM (L154); 1 mL of Gamborg Vitamin solution [1000×] (G219); the solution was brought to volume with 180.132 mL of ddH₂O; the pH was adjusted to 5.8 with KOH.
   The media was autoclaved on AGAR cycle with MediaClave.

To create a culture media containing Sucrose, referred to as WPM-B5−30, the following protocol and volumes were used to create 1L of media:
800 mL of ddH₂O; 30 g of Sucrose (S9378); the solution was brought to volume with 181.132 mL of ddH₂O; the pH was adjusted to 5.8 with KOH.
   The media was autoclaved on AGAR cycle with MediaClave.

To create an infection culture media containing DKW, referred to as 0.5× DKW infection, the following protocol and volumes were used to create 1L of media:
800 mL of ddH₂O; 2.61 of DKW basal salt mixture (D190); the solution was brought to volume with 200 mL of ddH₂O; the pH was adjusted to 5.8 with KOH. The media was autoclaved on AGAR cycle with MediaClave.

To create a liquid culture media containing DKW and B5, referred to as DKW-B5−0 liquid, the following protocol and volumes were used to create 1L of media:

800 mL of ddH₂O; 5.22 g of DKW basal salt mixture (D190); 1 mL of Gamborg Vitamin solution [1000×] (G219); the solution was brought to volume with 199 mL of ddH₂O; the pH was adjusted to 5.8 with KOH.

The media was autoclaved on AGAR cycle with MediaClave.

To create another liquid culture media containing DKW and B5, referred to as DKW-B5−5 liquid, the following protocol and volumes were used to create 1L of media:
800 mL of ddH₂O; 5 g of Sucrose (S9378); 5.22 g of DKW basal salt mixture (D190); 1 mL of Gamborg Vitamin solution [1000×] (G219); the solution was brought to volume with 195.855 mL of ddH₂O; the pH was adjusted to 5.8 with KOH.
   The media was autoclaved on AGAR cycle with MediaClave.

To create another liquid culture media containing DKW and B5, referred to as DKW-B5-10 liquid, the following protocol and volumes were used to create 1L of media:
800 mL of ddH₂O; 10 g of Sucrose (S9378); 5.22 g of DKW basal salt mixture (D190); 1 mL of Gamborg Vitamin solution [1000×] (G219); the solution was brought to volume with 192.711 mL of ddH₂O; the pH was adjusted to 5.8 with KOH.
   The media was autoclaved on AGAR cycle with MediaClave.

To create another liquid culture media containing DKW and B5, referred to as DKW-B5−20 liquid, the following protocol and volumes were used to create 1L of media:
800 mL of ddH₂O; 20 g of Sucrose (S9378); 5.22 g of DKW basal salt mixture (D190); 1 mL of Gamborg Vitamin solution [1000×] (G219); the solution was brought to volume with 186.421 mL of ddH₂O; the pH was adjusted to 5.8 with KOH.
   The media was autoclaved on AGAR cycle with MediaClave.

To create another liquid culture media containing DKW and B5, referred to as DKW-B5−40 liquid, the following protocol and volumes were used to create 1L of media:
800 mL of ddH₂O; 40 g of Sucrose (S9378); 5.22 g of DKW basal salt mixture (D190); 1 mL of Gamborg Vitamin solution [1000×] (G219); the solution was brought to volume with 173.843 mL of ddH₂O; the pH was adjusted to 5.8 with KOH.
   The media was autoclaved on AGAR cycle with MediaClave.

To create another liquid culture media containing DKW and B5, referred to as DKW-B5−50 liquid, the following protocol and volumes were used to create 1L of media:
800 mL of ddH₂O; 50 g of Sucrose (S9378); 5.22 g of DKW basal salt mixture (D190); 1 mL of Gamborg Vitamin solution [1000×] (G219); the solution was brought to volume with 167.554 mL of ddH₂O; the pH was adjusted to 5.8 with KOH.
   The media was autoclaved on AGAR cycle with MediaClave.

To create another liquid culture media containing DKW and B5, referred to as DKW-BS-filter sterilize (FS) liquid, the following protocol and volumes were used to create 1L of media:

800 mL of ddH₂O; 30 g of Sucrose (S9378); 5.22 g of DKW basal salt mixture (D190); 1 mL of Gamborg Vitamin solution [1000×] (G219); the solution was brought to volume with 180.131 mL of ddH₂O; the pH was adjusted to 5.8 with KOH.
The media was filter sterilized.

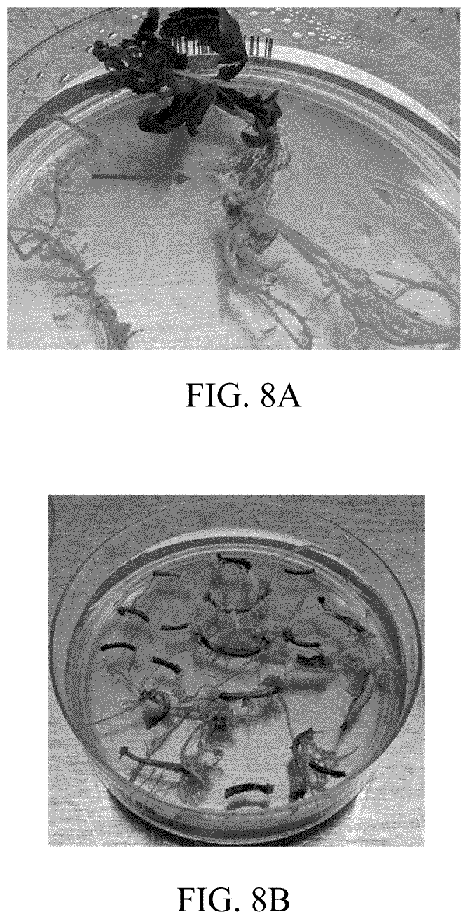

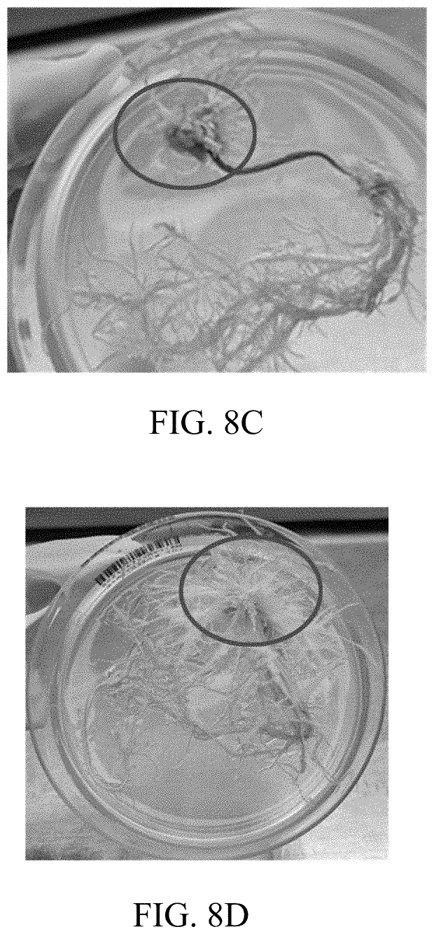

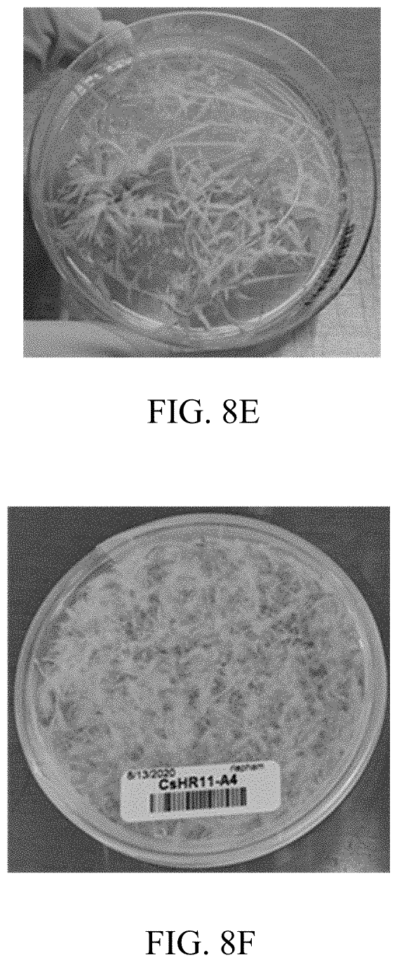

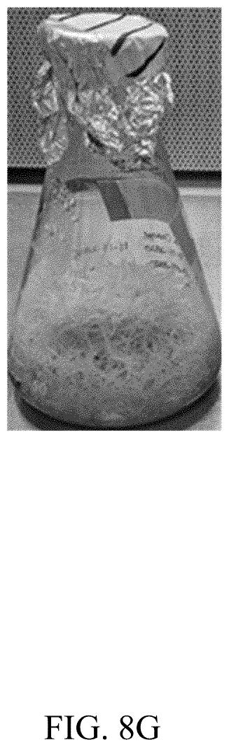

FIGS. 8A-8G illustrate cultured transgenic PCM tissue, consistent with the present disclosure. For example, FIG. 8A is an image demonstrating growth of transgenic PCM tissue from whole Cannabis seedlings in tissue culture, where the PCM tissue (arrow) form at the wound site. FIG. 8B is an image demonstrating growth of transgenic PCM tissue from Cannabis hypocotyl segments in tissue culture. FIGS. 8C-8D are images illustrating sub-cultured PCM tissue clones (in the circles). FIGS. 8E-8G are further images of sub-cultured PCM tissue clones. For example, FIG. 8F illustrates an example final Cannabis clone in solid media and FIG. 8G illustrates an example final Cannabis clone in liquid media.

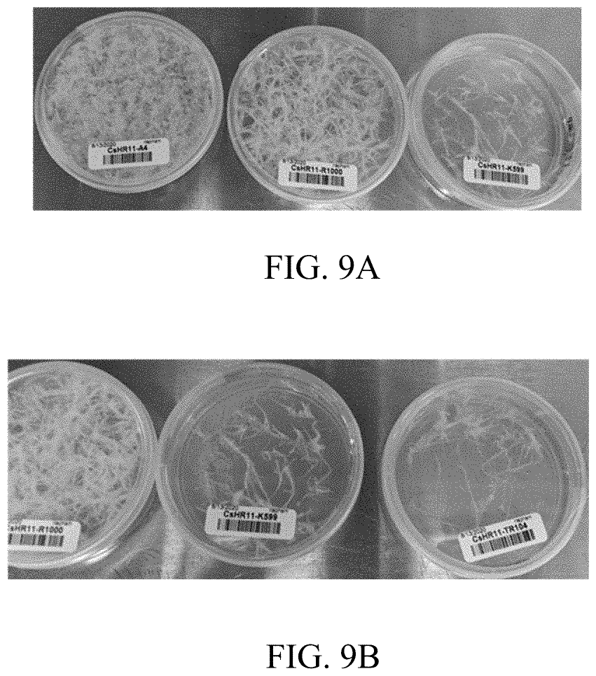

FIGS. 9A-9B illustrate cultured transgenic PCM tissue, consistent with the present disclosure. More particularly, FIGS. 9A-9B are images illustrating sub-cultured PCM Cannabis clones.

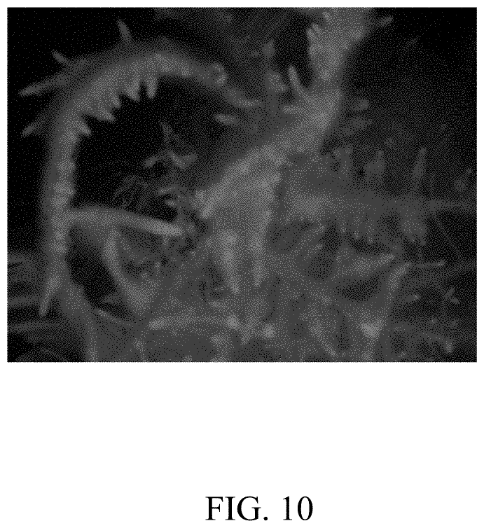

FIG. 10 illustrates example data of transgenic PCM tissue that produced a recombinant compound, consistent with the present disclosure. For example, FIG. 10 is an image showing successful transformation of a plant part with a bacterium strain carrying the plasmid vector of FIG. 6A, where the bacterium strain is the R. rhizogenes strain A4 (ATCC43057) having a first transgene that induces PCM formation and a second transgene including YFP.

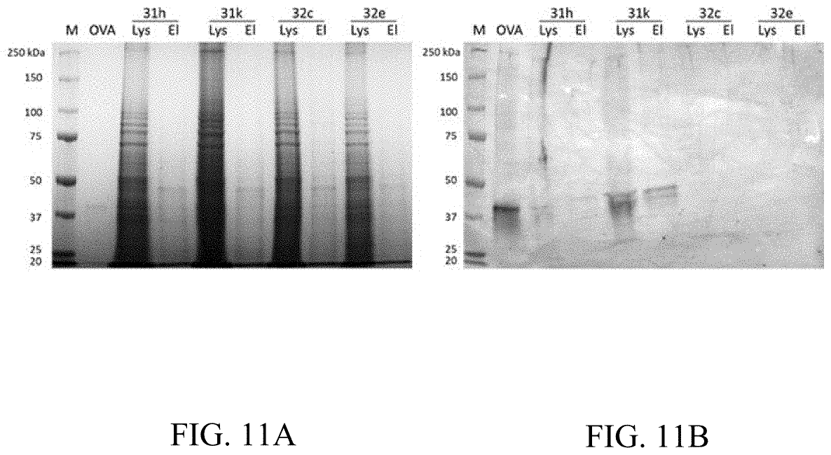

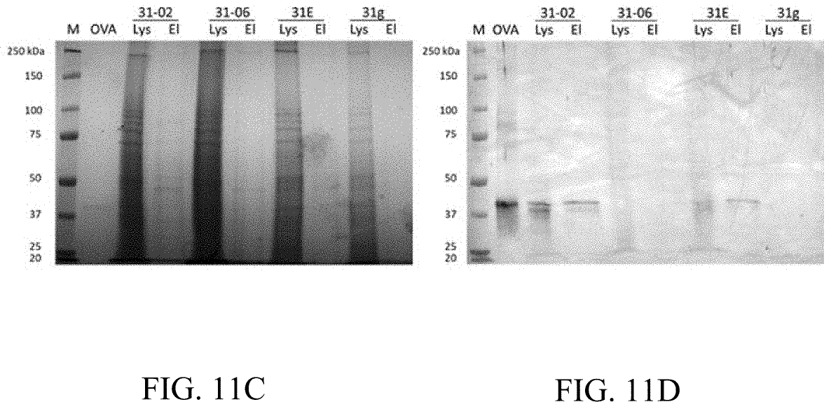

FIGS. 11A-11D illustrate example data showing production of a recombinant compound from PCM cultures, consistent with the present disclosure. More specifically, FIGS. 11A-11D are images showing successful production of ovalbumin from a Cannabis plants part transformed with a bacterium strain carrying the plasmid vector of FIG. 6B, where the bacterium strain is the R. rhizogenes strain A4 (ATCC43057) having a first transgene that induces PCM formation and a second transgene including ovalbumin. For example, FIGS. 11A and 11C illustrate results from a Coomassie blue protein gel analysis performed on PCM clones from the transformed Cannabis plant part and FIGS. 11B and 11D illustrate results from a Western Blot analysis performed on PCM clones from the transformed Cannabis plant part. Standard and well-known Coomassie blue protein gel and Western Blot techniques were used to perform the analysis. As may be appreciated, various well-known Coomassie blue protein gel and Western Blot techniques can be used. The Western Blot analysis (e.g., FIGS. 11B and 11D) shows successful production of ovalbumin (OVA) in lysate (LYS) and elution (EI) for multiple, independent PCM clones.

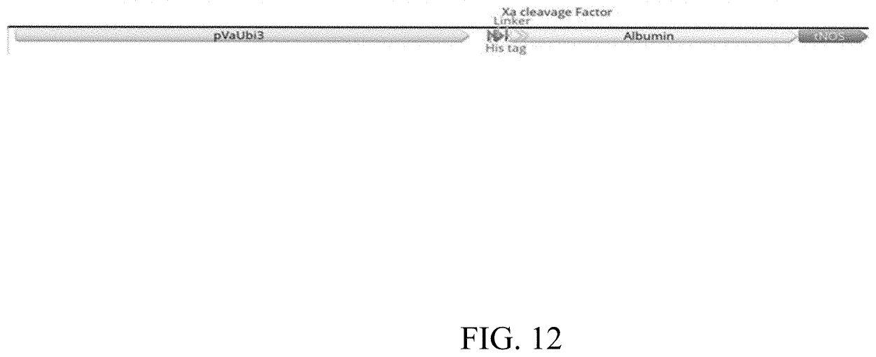

FIG. 12 illustrates an example expression cassette for the production of an albumin from a PCM culture, consistent with the present disclosure. A coding sequence for Gallus albumin or OVA comprises SEQ ID NO: 16 and the corresponding amino acid sequence comprises SEQ ID NO: 17, * indicates a stop codon. A coding sequence for Gallus albumin with N-terminal His tag and Xa cleavage factor for purification is illustrated by SEQ ID NO: 18 and the corresponding amino acid sequence is illustrated by SEQ ID NO: 19. A coding sequence the expression cassette illustrated in FIG. 12 which includes a Ubi3 promoter-albumin-NOS terminator is illustrated by SEQ ID NO: 20.

The Gallus DNA sequence coding for the Albumin protein (SEQ ID NO: 16), a codon optimized according to the codon bias used by the target Legume spp. (Glycine, Medicago, Vigna, Lens and Cicer), and cloned in binary vectors, are under the regulation of a strong promoter (Ubi-3) and a terminator (NOS) (FIG. 10, with polynucleotide sequences and cognate amino acid sequences shown in SEQ ID NOs: 16-20). The binary vector for R, rhizogenes transformation contains a Right and a Left T-DNA Border sequence, to allow Agrobacterium to deliver the DNA into the plant cells. As a control, a similar binary vector is constructed for the expression of the reporter gene YFP. For every experiment, the genes of interest are expressed in three different variants: (1) the sequence coding for the native protein; (2) the sequence with an endoplasmic reticulum retention (ER) signal (HDEL), to allow protein accumulation in the ER; and (3) the sequence with a N-terminal secretion signal, to allow the secretion of the desired protein in the spent medium. The expression cassettes permit easy modification by swapping transcription elements in order to optimize expression. Different Untranslated Regions (UTRs) are utilized to determine the effect on expression. Constitutive promoters and root specific promoters are selected for tissue-specific approaches.

Seeds from Legumes ssp. Glycine, Medicago, Vigna, Lens and Cicer are surface sterilized with chlorine gas for 16 hours. Seeds are either imbibed in sterile water overnight for infection using a half-seed transformation method or are placed on half strength MS solid medium pH 5.8 supplemented with 1% sucrose to germinate. Germination and seedling growth occur at 25 degrees C. with a 16 h light/8 h dark photoperiod. Germinated seedlings are transformed using a whole seedling wounding method described below.

Three strains of R. rhizogenes (obtained from ATCC) are grown on AB minimal media plates (supplemented with 50 mg/l kanamycin for selection of the binary plasmid). A bacterial inoculum is taken from freshly grown plates and inoculated into water containing acetosyringone for virulence gene induction. The bacterial suspensions are kept at room temperature in the dark for a number of hours before inoculating the plant tissue. Plant infection is performed by pricking the hypocotyls of 4-7-day old seedlings with the tip of a scalpel or needle before applying the bacterium suspension to the injured zone. Depending on the plant species, PCM tissue emerges from the wounded site 2 to 8 weeks after infection.

Infected hypocotyls developing PCM tissues are cut out from the seedling and placed on MS medium plus cefotaxime (500 ug/mL). After 10-14 days, clones with actively growing root tips are sub-cultured onto MS supplemented with 500 ug/mL cefotaxime and allowed to grow for another 10-21 days. This process is repeated every 10-21 days with the amount of cefotaxime in the media being reduced every other transfer from 500 to 300 to 100 ug/mL to obtain sterile root cultures absent of bacteria. As the clones grow, they are divided up across multiple media plates before eventually being transferred to media containing no antibiotics. To initiate cultures in liquid medium, 5-10 pieces (about 1 cm long) or an approximately 1 cm³ clump of each PCM clone is then transferred into a 1 L flask containing the following: 100 mL B5 medium supplemented with 3% sucrose, pH 5.8 at 20-25 degrees C. under dim light, on orbital shaker (90 rpm/min). Root clones are sub-cultured every 3 weeks using 1 g of root biomass for 100 mL of culture medium. These roots can be used to inoculate protein culture. For each clone generated, a root fragment can be checked for protein expression by SDS-PAGE and western blot analysis as described below.

Total protein accumulation from PCM cultures is initially assessed by the Bradford Assay and albumin expression is quantified by SDS-Page. A portion of the PCM culture is removed, homogenized in liquid nitrogen, and extracted in an appropriate buffer solution containing protease inhibitors. To perform the total protein quantification necessary to run the electrophoresis gel, negatively-charged Coomassie dye, which binds to the positively charged proteins, is added. This method results in a color change from red (absorbance at 465 nm) to brilliant blue (595 nm). The resulting absorbance is compared to a standard curve to quantify total protein. The proteins are diluted accordingly into the proper buffer solution and separated on a 12% (w/v) SDS polyacrylamide gel. When complete the gel is stained with Coomassie Brilliant Blue to visualize the total amount of albumin.

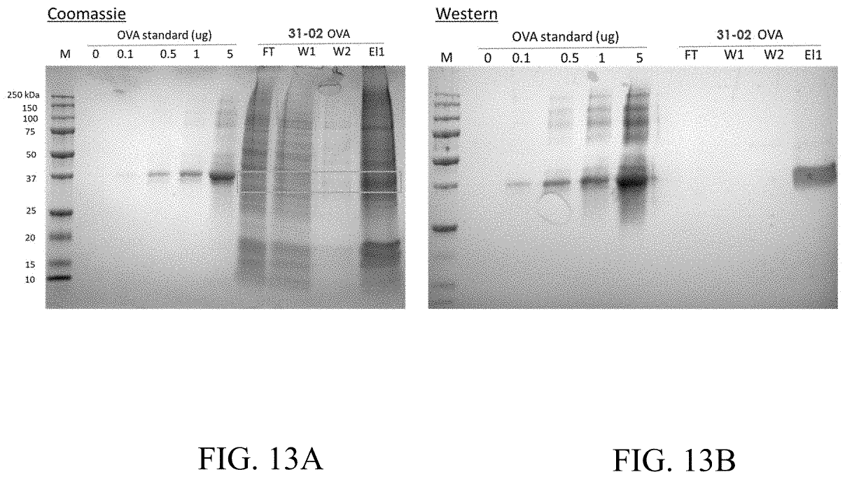

FIGS. 13-16C are images showing successful production of ovalbumin from a Cannabis plants part transformed with a bacterium strain carrying the plasmid vector of FIG. 6B, where the bacterium strain is the R. rhizogenes strain A4 (ATCC43057) having a first transgene that induces PCM formation and a second transgene including ovalbumin.

FIGS. 13A-13B illustrate example data showing production of ovalbumin from PCM cultures, consistent with the present disclosure. Polyhis-tagged ovalbumin was extracted from dry PCM tissue (e.g., 233.7 mg dry tissue from 31-02 (Ubi3 promoter)) and further purified using a nickel affinity column. Coomassie gel (FIG. 13A) and Western Blot (FIG. 13B) were run on the following samples: the protein ladder (lane 1), ovalbumin standards 0-5 μg (lanes 2-6), Ni column flow-through (lane 7), Ni column washes (lanes 8-9), Ni column eluent (lane 10). As shown in the Western Blot illustrated by FIG. 13B, antibody specificity is not seen in the flow-through or washes. This indicates that that polyhis-tagged ovalbumin was only present in the eluent. It is also observed that the anti-ovalbumin antibody selectively binds to ovalbumin and not the background proteins. As shown in the images, flowthrough (FT) is 10× diluted, first wash is represented by W1, second wash is represented by W2, and the first 100 ul of eluent is represented by E11.

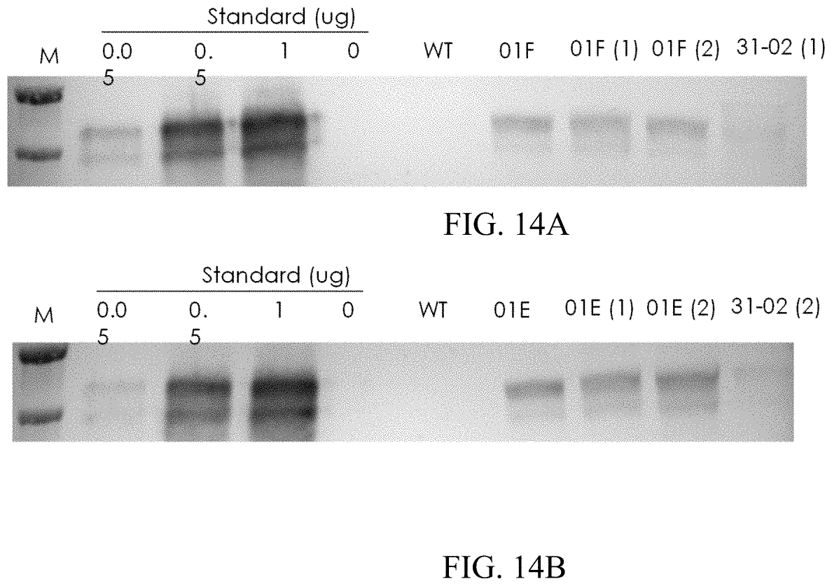

FIGS. 14A-14B illustrate further example data showing production of ovalbumin from PCM cultures, consistent with the present disclosure. In such embodiments, the polyhis-tagged ovalbumin was extracted from dry PCM tissue (e.g., 233.7 mg dry tissue from 31-02 (Ubi3 promoter)) but not purified or enriched with a nickel affinity column and lysate was run directly. The PCM tissue was screened for ovalbumin in PCM to find the best for equivalency analysis.

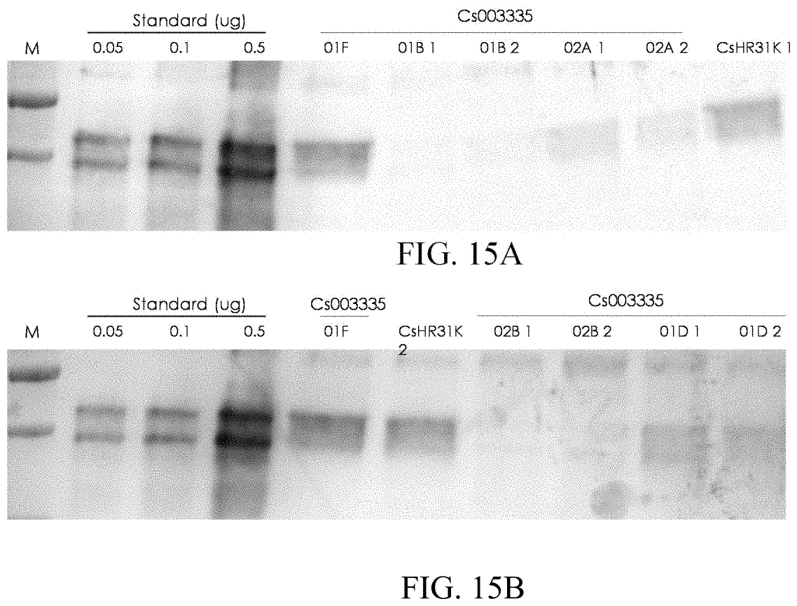

FIGS. 15A-15B illustrate further example data showing production of ovalbumin from PCM cultures, consistent with the present disclosure. The PCM tissue was screened for ovalbumin in PCM tissue to find the respective best ovalbumin producing PCM cultures. In such embodiments, the polyhis-tagged ovalbumin was extracted from dry PCM tissue into buffer 5 and lysate was run directly for Western Blot. FIGS. 15A-15B show the Western Blot results of two best PCM tissues and shows similar levels of ovalbumin between clones. As shown, there was high levels of 01F, 31K, and 01D, and lower levels of 01B, 02A, and 02B, respectively.

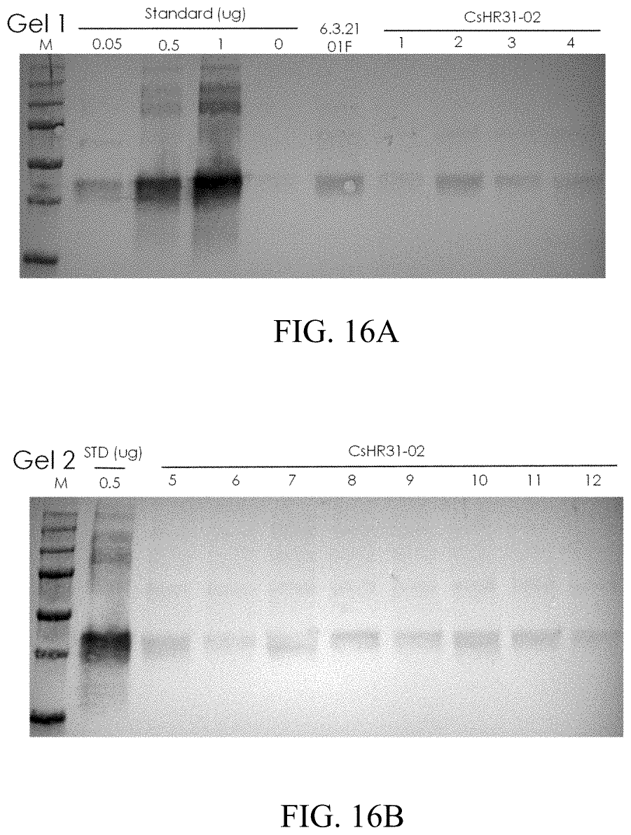

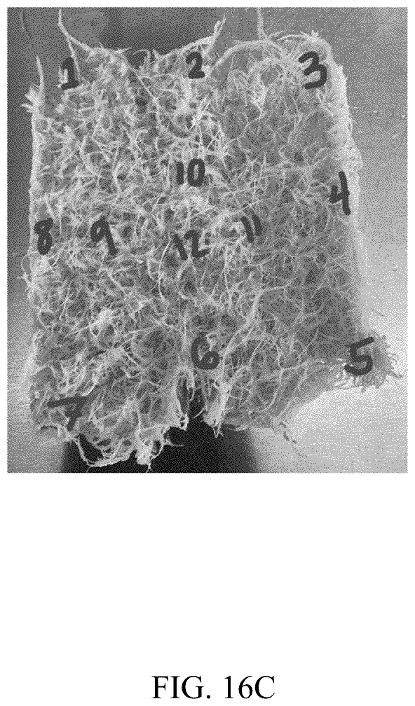

FIGS. 16A-16C illustrate further example data showing production of ovalbumin from PCM cultures, consistent with the present disclosure. Some experimental embodiments were directed to comparing ovalbumin quantities by position. FIGS. 16A-16B illustrates the Western Blot results of example PCM tissue at the different positions of the PCM culture, with the different positions illustrated by FIG. 16C.

Various experiments were conducted to assess ovalbumin clone yields and to approximate yield from screening clones for ovalbumin. Table 1 below illustrates example yield results.

TABLE 1

| | Average Yield | | |
|---|---|---|---|
| | (ug of albumin/g | Standard | # of |
| Clone | of dry tissue) | Deviation | samples |
| 1 | 344.1 | 209.6 | 6 |
| 2 | 70.3 | 39.9 | 3 |
| 3 | 99.6 | 70.3 | 12 |
| 4 | 86.7 | 51.7 | 6 |
| 5 | 64.9 | 13.1 | 2 |

Several independent PCMs were generated that produced ovalbumin across multiple experiments, indicating that the recombinant ovalbumin transgene is stable in the PCMS and the production of ovalbumin is reproducible. The PCMS produced ovalbumin concentrations ranging from about 65 ug/g of dry tissue to abound 344 ug/g of dry tissue.

Various embodiments were directed to deriving betalains from a PCM. Betalains, as used herein, include tyrosine-derived pigments, which can be red, red-violet, violet, yellow, orange, and yellow-orange. Betalains include betacyanins, which are red to violet betalain pigments, and betaxanthins, which are yellow to orange betalain pigments. Example betacyanins include betanin, isobetanin, probetanin, and neobetanin. Example betaxanthins include vulgaxanthin, miraxanthin, portulaxanthin, and indicaxanthin. Betalains are produced by converting tyrosine in the plant or other organism to L-3,4-dihydroxyphenylalanine (L-DOPA) and then converting L-DOPA to the different betalains through different enzymatic pathways.

In various experiments, a plant tissue is transformed using a bacterium strain that causes PCM formation, and is co-transformed or re-transformed to produce the betalain via a nucleotide sequence encoding an enzyme associated with production of a betalain. In various experiments, plant parts are co-transformed to induce PCM formation and production of the betalain. In other experiments, two transformations are performed: the first transformation causes the formation of the PCM, and the PCM tissue is further transformed to produce the betalain. The enzyme can be associated with the pathway for converting tyrosine to the betalain. Tyrosine can be naturally synthesized by the plant. In some embodiments, the nucleotide sequence can additionally encode a reactant, such as tyrosine. For example, tyrosine can be upregulated or overexpressed due to the transformation.

The betalain can be referred to as a secondary metabolite which is produced and/or increased in production due to transformation and production of the enzyme. The enzyme can comprise a plurality of enzymes including L-DOPA, dihydroxyphenylalanine (DOPA) 4,5-dioxygenase (DODA), Cytochrome P450 (CYP76AD1), CYP76AD6, glucosyltransferase, among other enzymes and combinations thereof. In some embodiments, a combination of multiple enzymes are expressed, such as DODA, CYP76AD1, and glucosyltransferase. Example glucosyltransferase include, without limitation, betanidin-5-O-glucosyltransferase and cyclo-DOPA-5-O-glucosyltransferase. In some embodiments, the three enzymes of DODA, CYP76AD1, and glucosyltransferase can create a heterologous pathway with a natively produced reactant of tyrosine to produce betanin. In other embodiments, two enzymes can be expressed. For example, the enzymes of DODA and CYP76AD1 can create a heterologous pathway with a natively produced reactant of tyrosine to produce betanidin. As another example, enzymes of DODA and CYP76AD6 can create a heterologous pathway with a natively produced reactant of tyrosine to produce betaxanthins. The different enzymes can be separated or linked by 2A self-cleaving peptides, such as P2A, F2A, T2A, and E2A. The 2A self-cleaving peptides induce ribosomal skipping during translation, thereby assisting in generating the separate enzymes during translation by causing the ribosome to fail at making a peptide bond.

In various experimental embodiments, the expression constructs illustrated by FIGS. 6D-6I were used to transform plant parts to induce PCM formation and production of betalains, such as the production of betanidin, betaxanthin, and/or betaxanthin. In some experiments, Cannabaceae plant parts were transformed, such as Cannabis plant parts, however embodiments are not so limited.

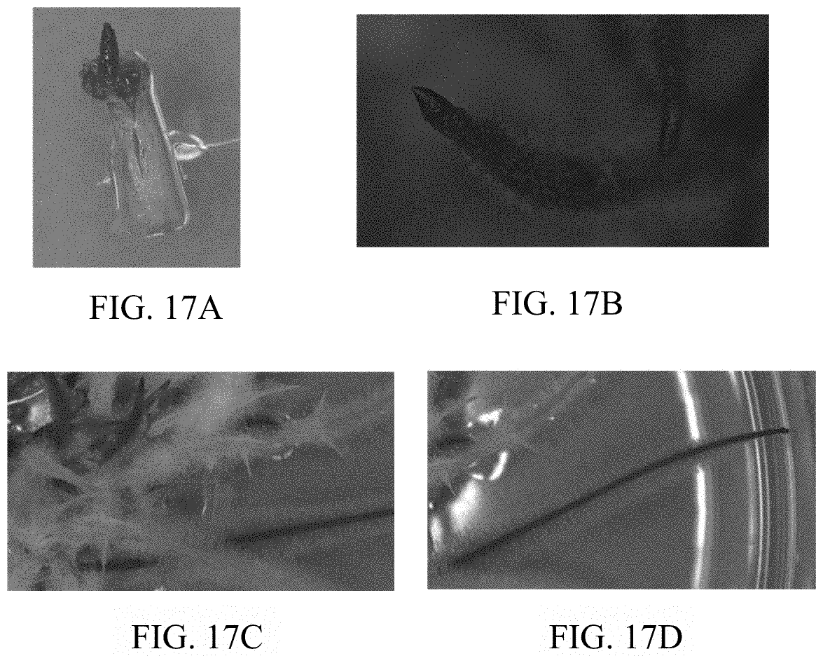

FIGS. 17A-17D illustrate example images of PCM cultures producing betacyanin, consistent with the present disclosure. In some experiments, Cannabis hypocotyls were co-transformed to generate PCMs and produce betalains using an R. rhizogenes strain A4 transformed with the plasmid vector 657 illustrated by FIG. 6E. The resulting PCMs produced a betacyanin, specifically, betanidin. FIG. 17A is an image showing the Cannabis hypocotyl segment forming a PCM that is producing the betacyanin. FIGS. 17B-17D are images showing a Cannabis PCM explant producing the betacyanin. The betacyanin is seen in the root tip, primary root, and root hairs. In FIGS. 17C-17D, multiple transgenic events are shown with different levels of betacyanin production.

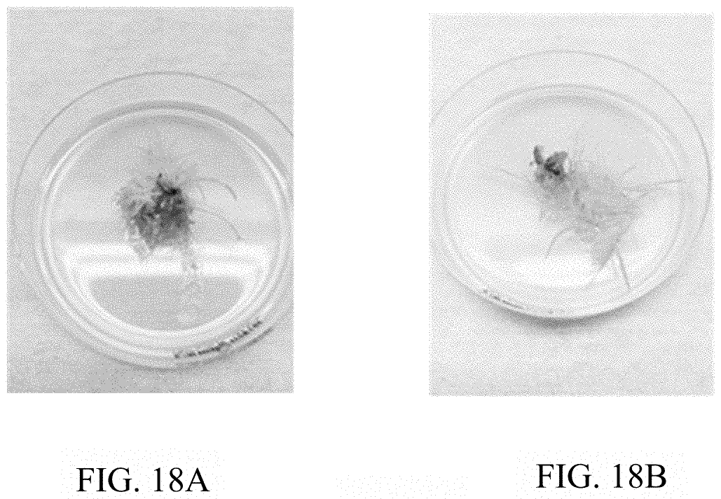

FIGS. 18A-18B illustrate example images of PCM cultures producing betacyanin at different levels, consistent with the present disclosure. In some experiments, Cannabis whole seedlings were co-transformed to generate PCMs and produce betalains using an R. rhizogenes strain A4 transformed with the plasmid vector 657 illustrated by FIG. 6E. FIGS. 18A-18B are images showing Cannabis PCM explants producing different levels of betacyanin after transforming whole seedlings.

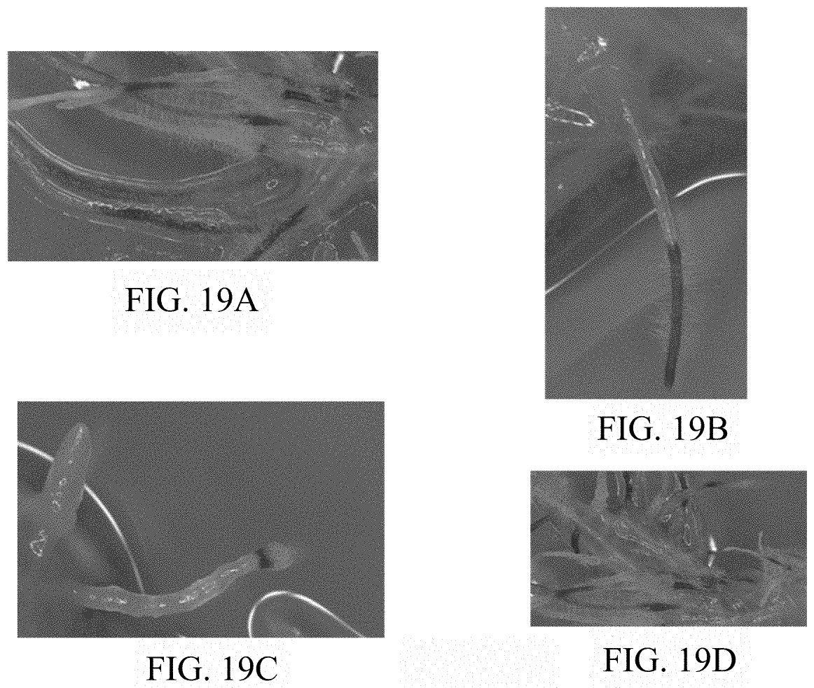

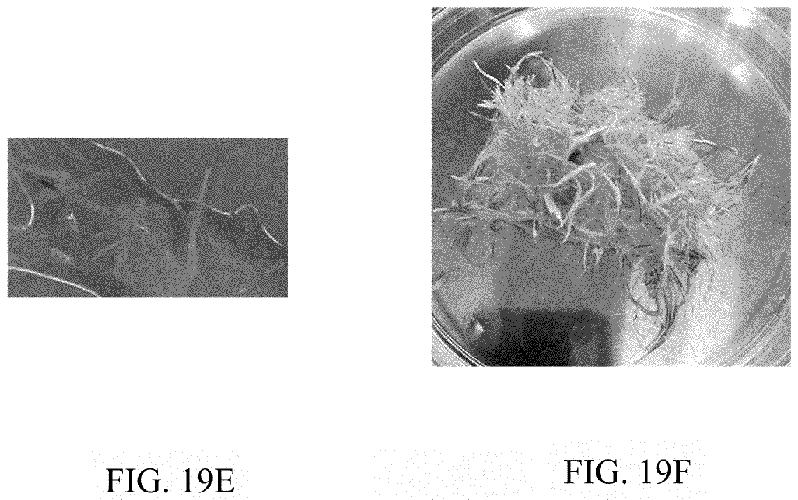

FIGS. 19A-19F illustrate example images of PCM cultures producing betacyanin at different levels, consistent with the present disclosure. In some experiments, the Cannabis PCMs were re-transformed with the plasmid vector 650 illustrated by FIG. 6B. FIGS. 19A-19F are images of the PCM tissues that were re-transformed and that produced different levels of betacyanin. The betacyanin, e.g., betanidin, is produced in roots, root tips, root hairs, and wounding sites.

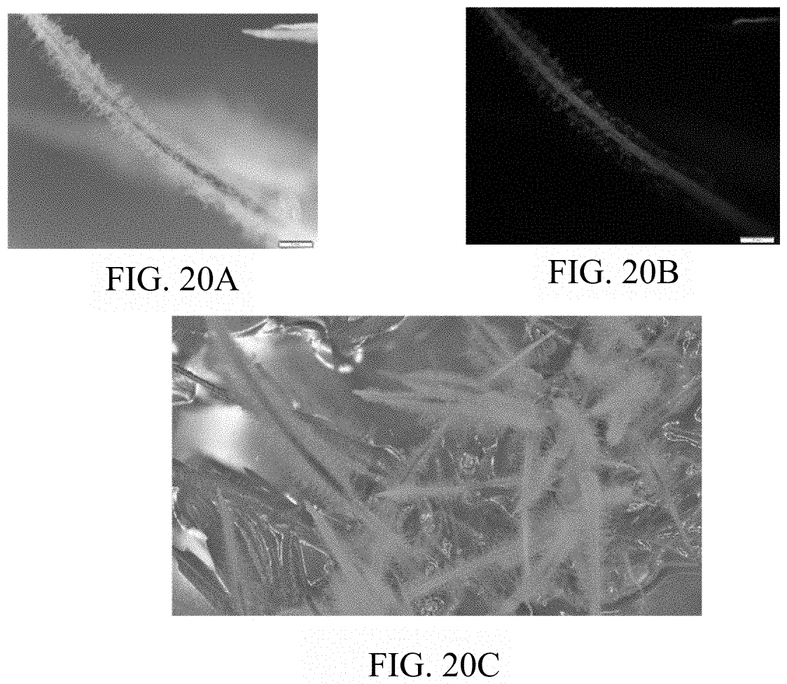

FIGS. 20A-20C illustrate example images of PCM cultures producing betanidin and betaxanthin, consistent with the present disclosure. In some experiments, Cannabis plant parts were transformed to generate PCMs using a R. rhizogenes strain A4 and then re-transformed to produce betalains using a disarmed R. rhizogenes strain A4 transformed with the plasmid vector 671 illustrated by FIG. 6H. The resulting PCM cultures produced multiple betalains including betanidin and betaxanthin. FIG. 20A is an image of the resulting PCM transformed with the plasmid vector 671 imaged in white light, and FIG. 20B is an image of the PCM of FIG. 20A imaged in fluorescent light under excitation at 488 mm. The first transformation included a protocol involving a first bacterium strain as described above (e.g., culturing to form PCM cultures), and the second or retransformation included exposing the formed PCM tissue to the second bacterium strain, such as 18r12. Other types of bacterium strains can be used as the second bacterium strain, including GV3101, AGL1, and EHA105. FIG. 20C is an image verifying the presence of betanidin and betaxanthin in the transformed PCM tissue.

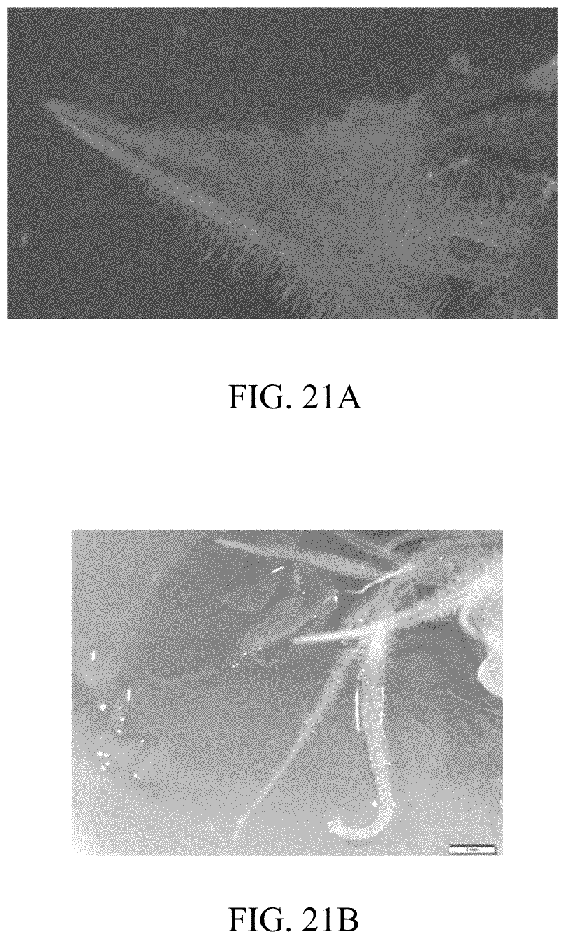

FIGS. 21A-21B illustrate example images of PCM cultures producing betaxanthin, consistent with the present disclosure. In some experiments, Cannabis plant parts were transformed to express PCM phenotype using a first R. rhizogenes strain A4 and then the PCM tissues were re-transformed to produce a betalain using a disarmed R. rhizogenes strain A4 strain, e.g., 18r12, transformed with the plasmid vector 665 illustrated by FIG. 6G. FIGS. 21A-21B are images of plant PCM tissue re-transformed and producing betaxanthin.

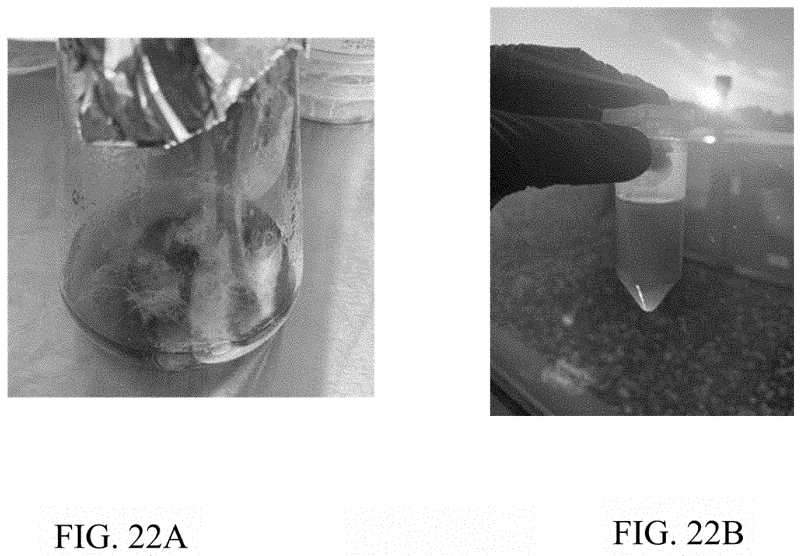

FIGS. 22A-22B illustrate example images of betalains in liquid from PCM cultures, consistent with the present disclosure. In some experiments, the Cannabis plant parts were transformed to form PCMs using a first bacterium strain and then re-transformed to produce betalains using plasmid vector 652 of FIG. 6D and a second bacterium strain as described above, and the resulting PCM culture secreted or otherwise presented the betalain into the liquid media. FIGS. 22A-22B are images showing the betalain present in the liquid media after the Cannabis PCMs were re-transformed with the plasmid vector 652.

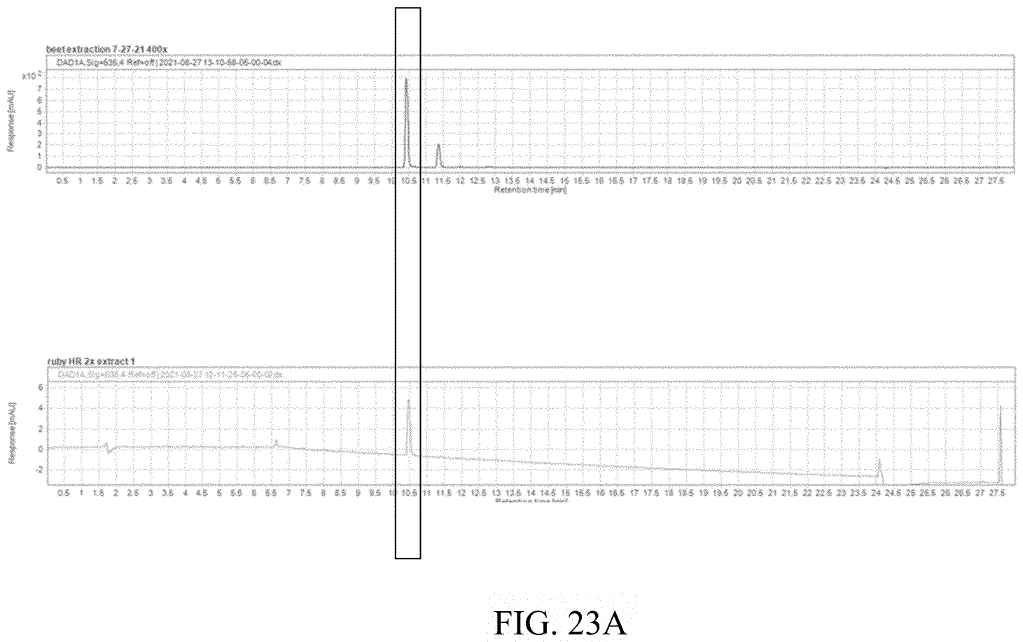

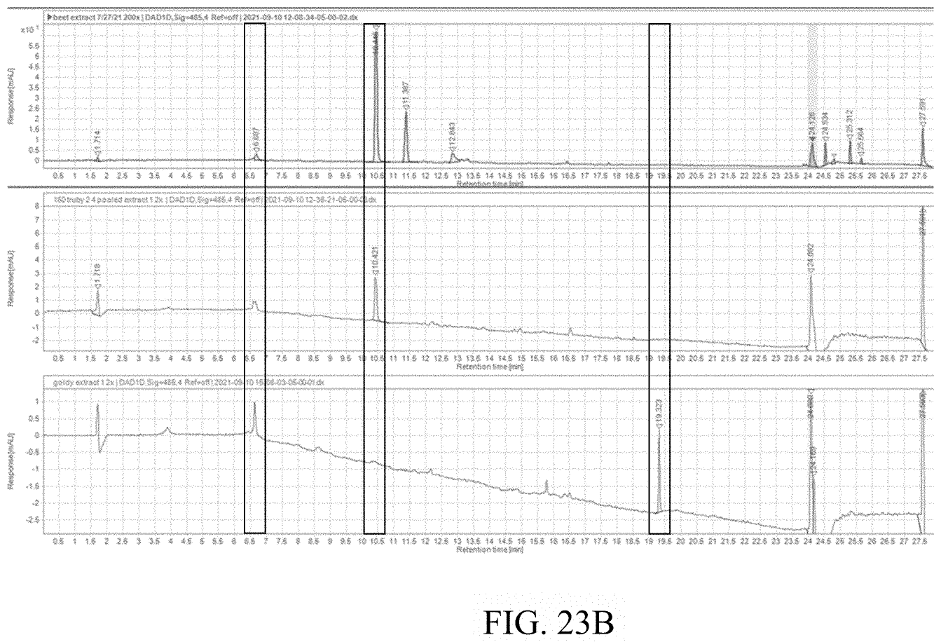

FIGS. 23A-23B illustrate example experimental results from PCM cultures producing betalains, consistent with the present disclosure. FIG. 23A illustrates a chromatogram of chemistries derived from a wild-type beet tissue extract on the top graph as compared to and lined up with a Cannabis PCM line on the bottom graph. The box illustrates a betalain, such as a betanin and/or betanidin. As shown by the top graph of FIG. 23A, the wild-type beet standard has a peak for betanin at around 10.446. The Cannabis PCM was generated using the plasmid vector 672 illustrated by FIG. 6I and has a similar peak around 10.446, as shown by the bottom graph of FIG. 23A. FIG. 23B illustrates a chromatogram of chemistries derived from a wild-type beet tissue extract on the top graph as compared to a first Cannabis PCM in the middle graph that was generated using the plasmid vector 671 of FIG. 6H, and a second Cannabis PCM in the bottom graph that was generated using the plasmid vector 665 of FIG. 6G. As shown by the top graph of FIG. 23B, the wild-type beet standard has peaks for betaxanthin at around 6.687, betanin at around 10.446, and isobetanin at around 11.387. As shown by the middle graph of FIG. 23B, the first Cannabis PCM has a peak around 10.446 that corresponds with betacyanin. As shown by the bottom graph of FIG. 23B, the second Cannabis PCM has a peak around 6.687 that corresponds with betaxanthin.

Embodiments in accordance with the present disclosure are not limited to transforming Cannabaceae plant parts and/or to producing albumins and/or betalain. Albumins, betalains, and/or other PCM-derived compounds can be produced in plant parts of other plant species by optionally identifying a bacterium strain to transform the plant part, and optionally by designing and generating a plasmid vector that includes heterozygous sequence encoding the PCM-derived compound, transforming the bacterium strain with the plasmid vector, and infecting the plant part of the plant with the transformed bacterium strain or otherwise contacting the plant part with the plasmid vector. The PCM-derived compound can be produced in various different plant species and include various types of PCM-derived compounds by designing an expression construct to infect the plant species, developing a tissue culture and transformation methodology, and transforming the plant part to produce the betalain.

An example process for designing the expression construct includes the following: 1) cloning and sequencing the gene of interest (e.g., the target enzyme(s) for a betalain) into an entry vector, 2) optionally adding a purification tag for isolation, 3) cloning into a binary vector containing a selectable marker and/or reporter gene (e.g., YFP), 4) testing a variety of promoters for protein expression using protoplasts, tissue infiltration, and/or transient transformation assays, 5) selecting a promoter which shows the highest expression among the tested variety of promoters in the target species to drive the expression of the gene of interest, and 6) introducing the binary vector into a Rhizogenes strain which gives the highest frequency of PCM formation (see below) among a set of strains or into another bacterium strain that does not induce PCM formation (e.g., 18r12).

An example process for developing a tissue culture and transformation methodology includes the following: 1) infecting the target plant species with a variety of wild-type Rhizogenes strains, 2) determining the transformation frequencies of the variety of wild-type Rhizogenes strains based on the number of explants which form PCM tissue out the total explants, 3) testing a variety of tissue from the target species (cotyledon, hypocotyl, stem, leaf, root, immature embryo, etc.) to determine which tissue is the most amenable to PCM formation, 4) subculturing formed PCMs and optimize media formulations to maximize growth and biomass accumulation via repeat adjustment and testing, 5) optimizing the subculturing technique, medias, and timing via repeat adjustment and testing, 6) determining which Rhizogenes strain produces the highest transformation frequency and produces PCM clones which grow well and for long periods of time in tissue culture, 7) determining the optimal selectable marker to be used for selection of the gene of interest by performing kill curve assays using a number of different selection agents on isolated PCM clones, 8) introducing the optimized vector (see above) into the selected Rhizogenes strain and infect tissue most amenable to transformation in target plant species, and 9) subculturing and selecting for PCM clones which express the gene of interest.

Various experimental embodiments were directed to generating PCM using adult plant tissue, such as petioles, internodes, leafs, among other tissue. In various experimental embodiments, hop plant tissue and Cannabis plant tissue was used to generate PCMs.

In some embodiments, the hop plant tissue was prepared as described above. In other experimental embodiments, hop plant tissue was prepared as follows.

The following methodology was used to surface sterilize clone-based hop plant tissue. Petioles or stem sections (with or without nodes) were collected from young shoots of hops plants (PI 558598) growing in the greenhouse (18 hour day length; temperature range 22-26 degrees C.). Petioles or stem sections were cut to a length between 3 cm and 7 cm. Petioles or stem sections cut between 3 cm and 7 cm were then briefly rinsed in water and placed into a 50 mL conical tube containing 30 mL of ddH2O. The ddH2O was discarded from the 50 mL conical tube. 30 ml of a 70% ethanol solution was added to the 50 mL conical tube and the tube was placed into a rotary shaker for 3 minutes, and the ethanol was discarded. 30 mL of a 2% sodium hypochlorite+0.01% Tween 20 solution was then added to the 50 mL conical tube and the tube was placed into a rotary shaker for 10 minutes. In a flow hood, shoots were washed in sterilized distilled water at least three times.

The surface-sterilized hop plant tissue was then used to propagate the hop plant in tissue culture using the following methodology. Several Phytocon containers (PhytoTech Labs; Lenexas, Kans.) were prepared with 25 mL of HL01 MSBS+Cef500 media. In a flow hood, a sterile scalpel was used to cut the surface sterilized hop plant stems (with nodes) to create node containing stem sections with a 1-2 cm of stem below the node and 0.5-1 cm of stem above the node. The node stem sections were placed vertically into the HL01 MSBS+Cef500 media with the 1-2 cm stem section going into the media such that the node is at or above the surface of the media. Propagating plants were transferred to new media every 2 weeks, or as necessary. Explants were grown in an incubator with 18 hour day length at 23 degrees C. The above described sterile technique was used when harvesting leaves, petioles, and internodes for transformation, as further described herein.

The bacteria was prepared seven days prior to infection by transforming a bacteria A. rhizogenes ATCC15834 strain with a plasmid containing the coding sequence for a fluorescent protein. However, embodiments are not so limited. In some experiments, the plasmid vector 674 illustrated by FIG. 6J was used, which encodes for YFP. The agrobacterium was streaked onto AB media+KAN50 media plates (e.g., plates with AB media+KAN50) and the media plates were incubated at 28 degrees C. until the day of the experiment. At least 3 days prior to infection, a loopful of the bacteria from the agrobacterium streak plate is taken and suspended in 1 mL of sterile water contain 100 μM acetosyringone. The bacterial suspension was placed in the dark at room temperature until initiation of the experiment.

In various experiments, hop leaf blade explants were prepared prior to co-cultivation . To achieve high quality leaves for transformation, leaves were harvested from tissue culture propagated plants. In a flow hood, leaf bales from the plants were removed and placed in petri dishes containing sterile ddH2O. A sterile scalpel was used to wound the leaves and create leaf sections 1-2 cm² in size. Special attention was paid to induce wounds in the leaf veins and wounds at the point in which the leaf attaches to the petiole, as calli, and subsequently PCMs form at these positions. All explants were kept in ddH2O until transformation to prevent desiccation.

In various experiments, hop petiole explants were prepared prior to co-cultivation. Petioles can be sourced from tissue culture grown plants or greenhouse grown plant. When sourcing petioles from tissue culture, the above-described sterile technique is used when harvesting the petioles. When petioles are sourced from the greenhouse, the surface sterilizing technique may damage tissue at the ends of the petioles, e.g., about 0.5 cm, and the damaged ends of the petioles, indicated by a lack of chlorophyll, are removed with a sterile scalpel. The petioles are then placed in sterile ddH2O, and a sterile scalpel is used to cut the petioles into sections about 0.5-1.5 cm in length. All explants were kept in ddH2O until transformation to prevent desiccation.

In various experiments, hop internode explants were prepared prior to co-cultivation. Internodes can be sourced from tissue culture grown plants or greenhouse grown plant. When sourcing internodes from tissue culture, the above-described sterile technique is used when harvesting the petioles. When internodes are sourced from the greenhouse, the surface sterilizing technique may damage tissue at the ends of the internodes, e.g., about 0.5 cm, and the damaged ends of the internodes are removed with a sterile scalpel. The internodes are then placed in sterile ddH2O, and a sterile scalpel is used to cut the internodes into sections about 0.5-1.5 cm in length. All explants were kept in ddH2O until transformation to prevent desiccation.

A variety of different infection methods were used. A first infection method included agrobacterium drip infection, which was found to be effective for inducing PCMs from hop petiole and internode explants. The following steps were performed for the agrobacterium drip infection method. A sterile forceps was used to transfer the explants to an empty sterile petri dish and a pipette was used to remove any freestanding ddH2O next to the explants. Alternatively, the explants were not moved and instead a pipette was used to remove the ddH2O from the plate. The sterile forceps were then used to form the explants into a pile. A pipette was used to add just enough agrobacterium solution to wet each of the explants, approximately 50-400 ul depending on how many explants were being transformed. To help ensure good coverage of the explants, in some experiments, standing agrobacterium solution at the bottom of the pile was pipetted up and deposited back onto the pile several times, varying the location each time. The petri dish plates were then wrapped with micropore tape and the plates were evacuated to 600 mbar for 15 minutes. The sterile forceps were then used to transfer the explants to a petri plate with HL01 MSBS media and form a pile of explants in the center of the plate. The petri dish was then wrapped in parafilm and placed into a dark incubator set to 23 degrees C. overnight followed by transferring the plate to an incubator set to 23 degrees C. and 18 hour light-6 hour dark conditions; or alternatively the plate was maintained in a dark incubator set to 23 degrees C. for the full duration of the co-cultivation step. The explants were co-cultivated for 1 to 3 days. After co-cultivation, the plate was visually examined for signs of agrobacterium overgrowth. If overgrowth was observed, the explants were washed using Antibiotic Rinse Solution 1. Sterile forceps were used to transfer explants to a petri dish containing 25 ml of Antibiotic Rinse Solution 1, the petri dish was wrapped in parafilm, and placed on an orbital shaker for 30 minutes set to 70 rpm. After shaking, sterile forceps were used to transfer explants to a petri dish contain 30 mL of sterile ddH2O and gently agitated the water for several seconds. Sterile forceps were then used to remove the explants from the sterile ddH2O and blot explants on sterile filter paper before transferring the explants to media containing antibiotics. The explants were transferred to media (HL01 MSBS+Cef500) containing antibiotics. And, explants were transferred to fresh media every 14 days, or as necessary.

A second infection method performed was an agrobacterium liquid plate infection. The agrobacterium liquid plate infection was effective for inducing PCMs from hop petiole, internode, and leaf explants. The following steps were performed for the agrobacterium liquid plate infection. A sterile forceps was used to transfer the explants to a petri dish containing 18 mL of sterile ddH2O and 2 mL of the prepared agrobacterium solution (10× dilution). The petri dish plate was wrapped with micropore tape and the plate was evacuated to 600 mbar for 15 minutes. The petri dish was placed on an orbital shaker for 30 minutes set to 70 rpm. Sterile forceps were used to transfer the explants to a petri plate with HL01 MSBS media. If the explants were from petioles and internodes, a pile of explants was formed in the center of the plate. If the explants were from leave blades, the explants were spread out on the media with the adaxial leaf surface of the explant in contact with the media. The petri dish was wrapped in parafilm and placed into a dark incubator set to 23 degrees C. overnight followed by transferring the plate to an incubator set to 23 degrees C. and 18 hours light-6 hours dark conditions; or alternatively the plate was maintained in a dark incubator set to 23 degrees C. for the full duration of the co-cultivation step. The explants were co-cultivated for 1 to 5 days. After co-cultivation, the plate was visually examined for signs of agrobacterium overgrowth. If overgrowth was observed, the explants were washed using Antibiotic Rinse Solution 1. For example, sterile forceps were used to transfer explants to a petri dish contain 25 mL of Antibiotic Rinse Solution 1, and the petri dish was wrapped in parafilm and place the petri dish on an orbital shaker for 30 minutes set to 70 rpm. After shaking, sterile forceps were used to transfer explants to a petri dish contain 30 mL of sterile ddH2O. The water was gently agitated for several seconds. The sterile forceps were used to remove the explants from the sterile ddH2O and blot explants on sterile filter paper before transferring the explants to media containing antibiotics. The explants were transferred to media (HL01 MSBS+Cef500) containing antibiotics, and to fresh media every 14 days, or as necessary.

Various experiments were conducted to assess the transformation efficiency and evaluate the same. For example, explants were screened for the presence of YFP using a YFP Xite™ Fluorescence Flashlight System (Nightsea; Lexington, Mass.) with the matching YFP barrier filter glasses twice a week starting 13 days after transformation. Explants identified as having YFP positive PCMs were then transferred to their own plate with HL01 MSBS+Cef500.

To create the Antibiotic Rinse Solution 1, the following protocol and volumes were used:
2 mL Cefotaxime [250mg/mL], 20 ml PPM, 988 mL ddH2O, and filter sterilized by passing the solution.

To create HL01 MSBS media, the following protocol and volumes were used to form 1L of media:
800 mL of ddH2O; 20 g of glucose (G386); 4.44 g of MS+5 Vitamins (M404); 1 g of MES (M825); the solution was brought to volume with 200 mL of ddH2O; the pH was adjusted to 5.6 by titration of KOH; and 6 g of Agar, Plant TC (A296).
   The media was autoclaved with AGAR cycle with MediaClave.

To create HL01 MSBS+Cef500 media, the following protocol and volumes were used to form 1L of media:
800 mL of ddH2O; 20 g of glucose (G386); 4.44 g of MS+5 Vitamins (M404); 1 g of MES (M825); the solution was brought to volume with 200 mL of ddH2O; the pH was adjusted to 5.6 by titration of KOH; and 6 g of Agar, Plant TC (A296). The media was autoclaved with AGAR cycle with MediaClave and 2 mL of Cefotaxime [250mg/mL] was added.

To create AB+Kan50 media, the following protocol and volumes were used to form 1L of media:

800 mL of ddH-2O; 30 g of sucrose, the solution was brought to volume with 200 mL of ddH2O; and 15 g of Molecular Grade Agar.

The media was as autoclaved with AGAR cycle with MediaClave. After autoclaving, the following were added: 50 mL of 20× AB salts, 50 mL of 20× AB Buffer, 1 mL of 50 mg/ml Kanamycin.

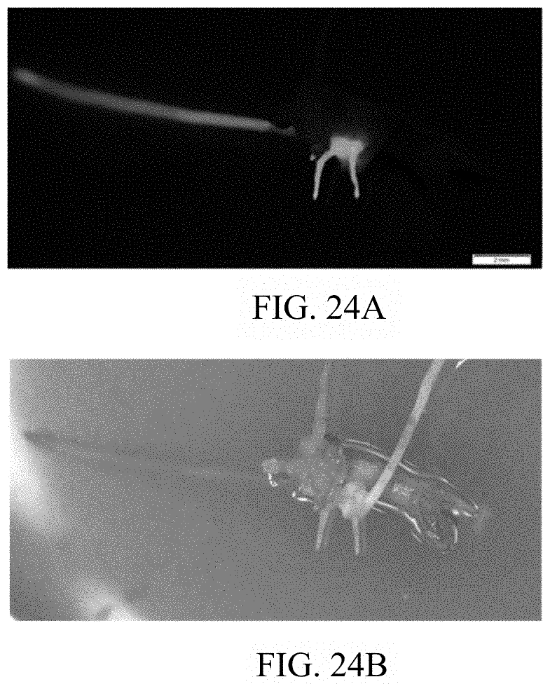

FIGS. 24A-24B illustrate example images of PCM cultures generated from hop plants, consistent with the present disclosure. As described above, hop petiole tissue from a hop plant (e.g., PI 558598, sourced from USDA) was transformed using a bacterium strain transformed to carry the plasmid vector 672 of FIG. 6J. FIG. 24A is an image of the PCM tissue of a PCM culture generated using hop petiole under an YFP filter, and FIG. 24B is an image under white light. As shown, the PCM culture is positive for YFP, demonstrating the transformation of the hop tissue.

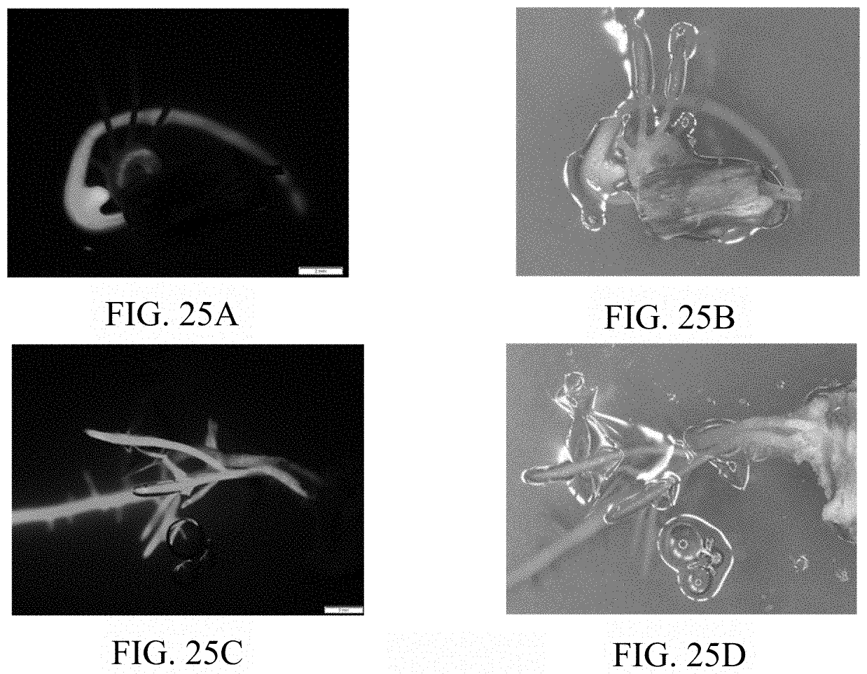

FIGS. 25A-25D illustrate example images of PCM cultures generated from hop plants, consistent with the present disclosure. As described above, hop internode tissue was transformed using a bacterium strain transformed to carry the plasmid vector 672 of FIG. 6J. FIG. 25A and FIG. 25C are images of PCM cultures generated using hop internodes under a YFP filter, and FIG. 25B and FIG. 25D are images under white light. As shown, the PCM is positive for YFP, demonstrating the transformation of the hop tissue.

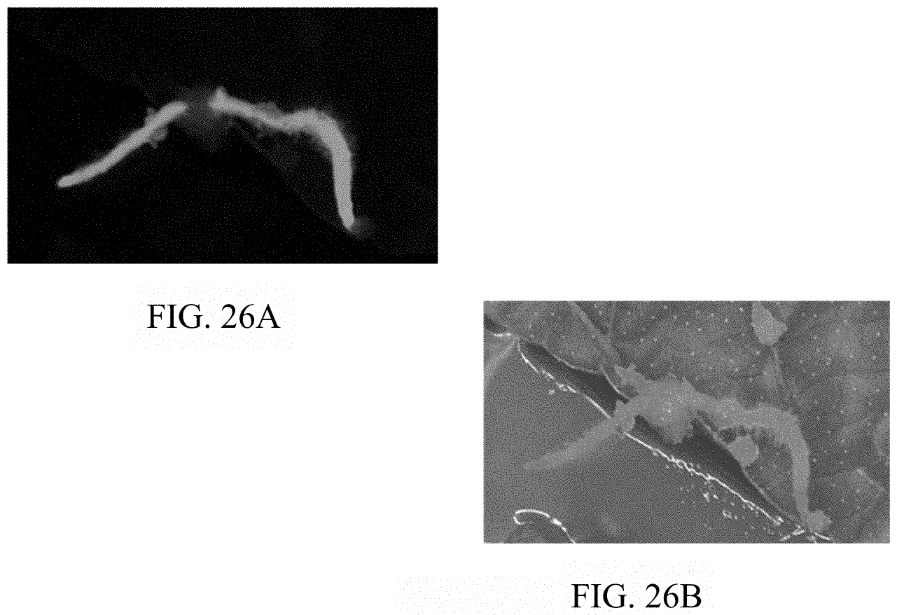

FIGS. 26A-26B illustrate example images of PCM cultures generated from hop plants, consistent with the present disclosure. As described above, hop leaf tissue was transformed using a bacterium strain transformed to carry the plasmid vector 672 of FIG. 6J. FIG. 26A is an image of the PCM tissue from the PCM culture generated using a hop leaf under an YFP filter, and FIG. 26B is an image under white light. As shown, the PCM is positive for YFP, demonstrating the transformation of the hop tissue.

Similar methods were be used to transform adult tissue of other plant species to generate PCMs. For example, Cannabis petioles, internodes, or leafs may be transformed. In other experimental embodiments, Cannabis plant tissue was prepared as follows.

The following methodology was used to surface sterilize clone-based Cannabis plant tissue. Petioles or stem sections (with or without nodes) were collected from young shoots of Cannabis plants (PL007.002) growing in the aeroponics (16 hour daylength; temperature range 20 degrees C.). Both older and younger petioles can be used for PCM generation. From experiments, it appears than younger petioles initiate PCM growth earlier than older petioles. Petioles were cut to a length between 3 cm and 7 cm. Petioles or stem sections cut between 3 cm and 7 cm were then rinsed in water and placed into a 50 mL conical tube containing 30 mL of ddH2O. The ddH2O was discarded from the 50 mL conical tube. 30 mL of a 70% ethanol solution was added to the 50 mL conical tube and the tube was placed into a rotary shaker for 3 minutes, and the ethanol was discarded. 30 mL of a 2% sodium hypochlorite+0.01% Tween 20 solution was then added to the 50 mL conical tube and the tube was placed into a rotary shaker for 10 minutes. In a flow hood, shoots were washed in sterilized distilled water at least three times.

The bacteria was prepared seven days prior to infection by transforming a bacteria A. rhizogenes ATCC15834 strain with a plasmid containing the coding sequence for a fluorescent protein, as previously described. In some experiments, the plasmid vector 674 illustrated by FIG. 6J was used, which encodes for YFP. The agrobacterium was streaked onto AB+KAN50 media plates (e.g., plates with AB media+KAN50) and the media plates were incubated at 28 degrees C. until the day of the experiment. At least 3 days prior to infection, a loopful of the bacteria from the agrobacterium streak plate is taken and suspended in 1 mL of sterile water contain 100 μM acetosyringone. The bacterial suspension was placed in the dark at room temperature until initiation of the experiment.

In various experiments, Cannabis petiole explants were prepared prior to co-cultivation. Petioles were sourced from vegetative plants grown in aeroponics, and the surface sterilizing technique may damage tissue at the ends of the petioles, e.g., about 0.5 cm, and the damaged ends of the petioles, indicated by a lack of chlorophyll, are removed with a sterile scalpel. The petioles are then placed in sterile ddH2O, and a sterile scalpel is used to cut the petioles into sections about 0.5-1.5 cm in length. All explants were kept in ddH2O until transformation to prevent desiccation.

A variety of different infection methods were used. A first infection method including agrobacterium drip infection. The following steps were performed for the agrobacterium drip infection method. A sterile forceps was used to transfer the explants to an empty sterile petri dish and a pipette was used to remove any freestanding ddH2O next to the explants. Alternatively, the explants were not moved and instead a pipette was used to remove the ddH2O from the plate. The sterile forceps were then used to form the explants into a pile. A pipette was used to add just enough agrobacterium solution to wet each of the explants, approximately 50-400 ul depending on how many explants were being transformed. To help ensure good coverage of the hemp explants, in some experiments, standing agrobacterium solution at the bottom of the pile was pipetted up and deposited back onto the pile several times, varying the location each time. The petri dish plate was then wrapped with micropore tape and the plate was evacuated to 600 mbar for 15 minutes. The sterile forceps were then used to transfer the explants to a petri plate with CsPCM media and form a pile of explants in the center of the plate. The petri dish was then wrapped in parafilm and placed into a dark incubator set to 23 degrees C. overnight followed by transferring the plate to an incubator set to 23 degrees C. and 18 hour light-6 hour dark conditions; or alternatively the plate was maintained in a dark incubator set to 23 degrees C. for the full duration of the co-cultivation step. The explants were co-cultivated for 1 to 3 days. After co-cultivation, the plate was visually examined for signs of agrobacterium overgrowth. If overgrowth was observed, the explants were washed using Antibiotic Rinse Solution 1. Sterile forceps were used to transfer explants to a petri dish containing 25 mL of Antibiotic Rinse Solution 1, the petri dish was wrapped in parafilm, and placed on an orbital shaker for 30 minutes set to 70 rpm. After shaking, sterile forceps were used to transfer explants to a petri dish contain 30 mL of sterile ddH2O and gently agitated the water for several seconds. Sterile forceps were then used to remove the explants from the sterile ddH2O and blot explants on sterile filter paper before transferring the explants to media containing antibiotics. The explants were transferred to media (CsPCM+Cef500) containing antibiotics. And, explants were transferred to fresh media every 14 days, or as necessary.

A second infection method performed was an agrobacterium liquid plate infection. The following steps were performed for the agrobacterium liquid plate infection. A sterile forceps was used to transfer the hemp explants to a petri dish containing 18 mL of sterile ddH2O and 2 mL of the prepared agrobacterium solution (10× dilution). The petri dish plate was wrapped with micropore tape and the plate was evacuated to 600 mbar for 15 minutes. The petri dish was placed on an orbital shaker for 30 minutes set to 70 rpm. Sterile forceps were used to transfer the explants to a petri plate with CsPCM media. A pile of explants was formed in the center of the plate. The petri dish was wrapped in parafilm and placed into a dark incubator set to 23 degrees C. overnight followed by transferring the plate to an incubator set to 23 degrees C. and 18 hours light-6 hours dark conditions; or alternatively the plate was maintained in a dark incubator set to 23 degrees C. for the full duration of the co-cultivation step. The explants were co-cultivated for 1 to 3 days. After co-cultivation, the plate was visually examined for signs of agrobacterium overgrowth. If overgrowth was observed, the explants were washed using Antibiotic Rinse Solution 1. For example, sterile forceps were used to transfer explants to a petri dish contain 25 mL of Antibiotic Rinse Solution 1, and the petri dish was wrapped in parafilm and placed on an orbital shaker for 30 minutes set to 70 rpm. After shaking, sterile forceps were used to transfer explants to a petri dish contain 30 ml of sterile ddH2O. The water was gently agitated for several seconds. The sterile forceps were used to remove the explants from the sterile ddH2O and blot explants on sterile filter paper before transferring the explants to media containing antibiotics. The explants were transferred to media (CsPCM+Cef500 media) containing antibiotics, and to fresh media every 14 days, or as necessary.

Various experiments were conducted to assess the transformation efficiency and evaluate the same. For example, hemp explants were screened for the presence of YFP using a YFP Xite™ Fluorescence Flashlight System (Nightsea; Lexington, Mass.) with the matching YFP barrier filter glasses twice a week starting 13 days after transformation. Explants identified as having YFP positive PCMs were then transferred to their own plate with CsPCM+Cef500 media.

To create CsPCM media, the following protocol and volumes were used to form 1L of media:
800 mL of ddH2O; 30 g of glucose (S9378); 4.43 g of MS Basal Salts+Vitamins (M519); the solution was brought to volume with 181.132 mL of ddH2O; the pH was adjusted to 5.8 by titration of KOH; and 6 g of Agarose(A6013).
   The media was autoclaved with AGAR cycle with MediaClave.

To create CsPCM+Cef500 media, the following protocol and volumes were used to form 1L of media:
800 mL of ddH2O; 30 g of glucose (S9378); 4.43 g of MS Basal Salts+Vitamins (M519); the solution was brought to volume with 179.132 mL of ddH2O; the pH was adjusted to 5.8 by titration of KOH; and 6 g of Agarose (A6013).
   The media was autoclaved with AGAR cycle with MediaClave and 2 mL of Cefotaxime [250mg/mL] was added.

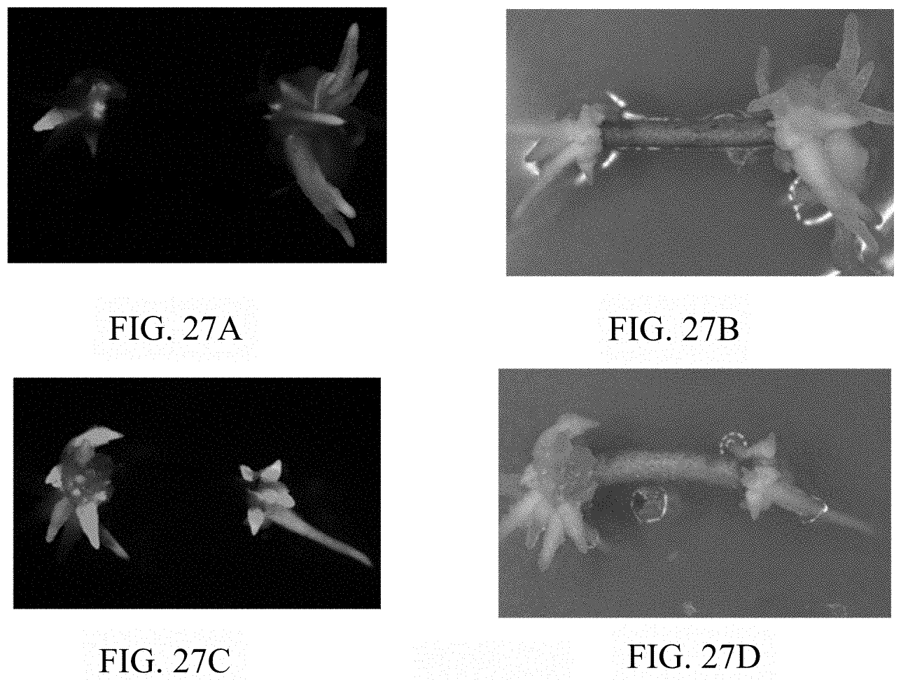

FIGS. 27A-27D illustrate example images of PCM cultures generated from Cannabis plants, consistent with the present disclosure. As described above, Cannabis petiole tissue was transformed using bacterium strain transformed to carry the plasmid vector 672 of FIG. 6J. FIG. 27A and FIG. 27C are images of PCM tissue from PMC cultures generated using Cannabis petioles under a YFP filter, and FIG. 27B and FIG. 27D are images under white light. As shown, the PCM is positive for YFP, demonstrating the transformation of the Cannabis tissue.

As described above, the PCM in experiments were generated from seeds, e.g., hypocotyl from seed, and adult plant tissue. Generating PCMs from adult plant tissue can provide advantages over seed generation, as the PCMs generate faster (e.g., in about an hour or less as compared to seven days), and is the explant generation and skills required is lower. Further, the safety risks are lower, as the sterilization in adult tissue is ethanol and bleach, as compared to H2SO4. It is believed that the resulting PCM may be more vigorous and allow for greater ease in selected transformed tissue. Furthermore, to generate a whole plant from the PCM can be simpler as the whole transgenic plant can be generated from the petiole, and even while still in tissue culture (e.g., no need to make seed).

Some experiment embodiments were directed to transforming Solanaceae plant parts to produce a PCM. For example, a solanum tuberosum plant part was transformed using an A. rhizogenes ATCC15834 strain, and under conditions described herein, and using a A. rhizogenes ATCC15834 strain containing a plasmid vector encoding for a plurality of enzymes associated with a betalain, such as the plasmid vector 672 illustrated by FIG. 6I.

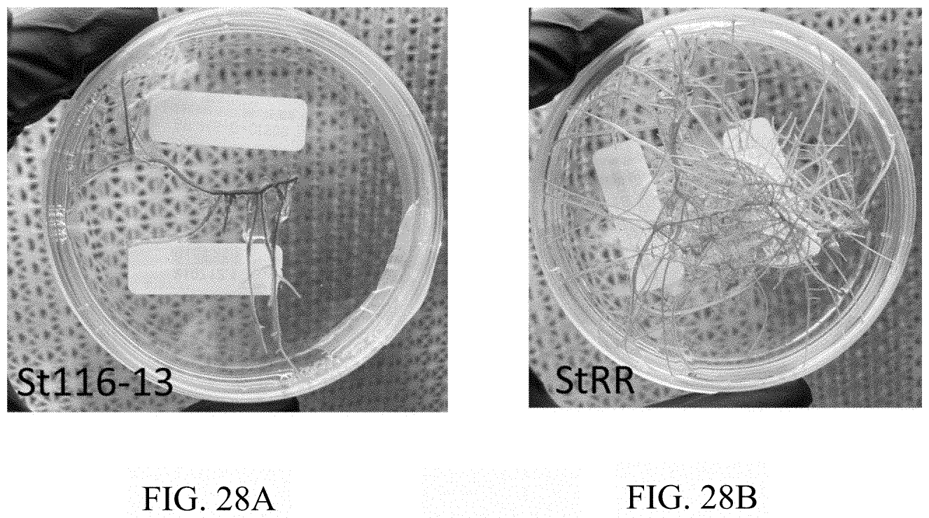

Some experiments were conducted that transformed the solanum tuberosum plant part to generate a PCM. FIGS. 28A-28B illustrate example images of PCM cultures generated from solanum tuberosum plants, consistent with the present disclosure. FIG. 28A illustrates a PCM culture generated from a first solanum tuberosum strain and FIG. 28B illustrates a PCM culture generated from a second solanum tuberosum strain. As previously described, genetic variability between strains and even clones of a strain results in different mass of PCM tissue produced in the PCM cultures. Some experimental embodiments were directed to assessing different biomass growth rates of PCM cultures generated from different clones. The resulting growth rates ranged from 1.5 to around 10 grams of biomass in two weeks of growth.

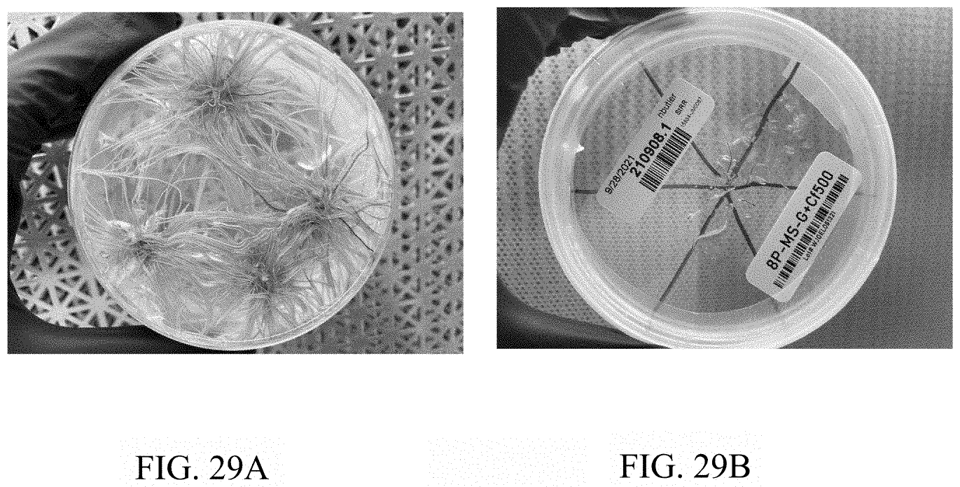

FIGS. 29A-29B illustrate example images showing betalain production in solanum tuberosum PCM cultures, consistent with the present disclosure. The solanum tuberosum plant parts were transformed using the plasmid vector 672, as noted above, and bacterium strain A. rhizogenes ATCC15834 transformed with the plasmid vector 672.

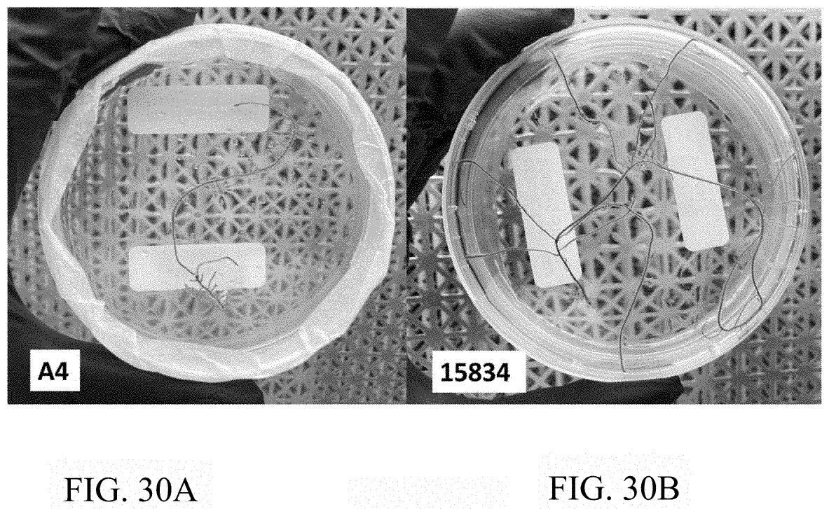

FIGS. 30A-30B illustrate example images of betalain production in solanum tuberosum PCM cultures using different bacterium strains, consistent with the present disclosure. For example, FIG. 30A illustrates betalain production in a PCM culture generated using A4 bacterium strain transformed with the plasmid vector 672 and FIG. 30B illustrates betalain production in a PCM culture generated using bacterium strain A. rhizogenes ATCC15834 transformed with the plasmid vector 672.

The Solanaceae plant parts were transformed using the below described protocols. In vitro solanum tuberosum plants were sub-cultured 3-5 weeks prior to use.

The bacterium stain was prepared by inoculating 25 mL of minimum growth (MG) media (in 50 mL sterile centrifuge tubes) supplemented with appropriate antibiotics (e.g. 50 mg/mL kanamyacin) loop/colony of A. rhizogenes carrying a binary plasmid (e.g. YFP reporter, incubated at 28 degrees C. with shaking for 2 days, OD600 around 0.5, and spun at 6000 RPM for 10 minutes in the large centrifuge at 4 degrees C. Supernatant and resuspended pellet was discarded in 25 mL MG media supplemented with 200 μM acetosyringone.

Solanaceae stem explant were then prepared by harvesting stems from 3-5 week old tissue-culture plants with thick (2-3 mm diameter) stems growing on a modified MS (MMS) media. The plant were cut at the internode below the lowest leaf to be harvested, and the container was covered in between harvests to prevent wilting. Excised shoot were placed on a sterile petri dish lid and stem internodes into 2-3 cm explants discarding any meristematic (nodal) tissue. The prepared stem explants were transferred to petri dish containing Agrobacterium solution and infected or co-cultivated. After 15-20 minutes in Agrobacterium solution, the infected stem explants were transferred to MS media with no antibiotics (100×15 mm petri dish) with 15-18 stem explants per petri dish, and sealed with parafilm and place in the dark (28 degrees C.) for 48 hours.

Regeneration was the performed following the co-cultivation. The explants were transferred to MS media petri dishes (100×15 mm) supplemented with 250 mg/L Cefotaxime and 150 mg/L Timentin, sealed with micropore tape, and then transferred to 16/8-hour light/dark (75 lumens, approximately 28 degrees C.) growth incubator, with the plates being transferred to fresh media every two weeks. PCMs growing were screened from the stem ends using fluorescent markers (e.g., YFP) and harvested as needed.

For transforming Solanaceae plant parts, to create the MS media, the following protocol and volumes were used to make a 1L solution of media:
600 mL ddH2O; 10 g Sucrose; 4.43 g MS Basal Salts+Vitamins (Phytotech, M519); the solution was brought to volume with 1000 mL ddH2O; the pH was adjusted to 5.7 with titration of KOH; and 3.58 g Gelzan™ (Phytotech, G3251) was added for a solid media.

To create the MMS media, the following protocol and volumes were used to make a 1L solution of media:
800 mL of ddH₂O; 25 g of sucrose; 4.45 g of MS Basal Salts+Vitamins (Phytotech, M519); the solution was brought to volume with 1000 mL ddH2O; the pH was adjusted to 5.7 with titration of KOH; and 7.5 g of Agar (Phytotech, A296) was added.
   The media was autoclaved on liquid cycle for 25 minutes, followed by adding 0.8 mL of Cefotaxime (250 mg/ml) and 01. mL of 6-BAP (1 mg/ml).

To create the MG salts (20×), the following protocol and volumes were used to make a 1L solution of media:
700 mL of ddH₂O; 20 g NH₄Cl; 6 g of MgSO4*7H₂O; 3 g of KCl; 0.2 g of CaCl2; 50 mg of FeSO4*7H2O; and the solution was brought to volume with 1000 mL of ddH2O.

To create the MG buffer (20×), the following protocol and volumes were used to make a 1L solution of media:
700 mL of ddH2O; 60 g of K2HPO4; 20 g of NaH2PO4; and the solution was brought to volume with 100 mL of ddH2O.

To create the MG media, the following protocol and volumes were used to make a 1L solution of media:
700 mL of ddH₂O; 5 g of glucose; and the solution was brought to volume with 1000 mL of ddH₂O; followed by adding 50 mL of 20× MG salts, 50 mL of 20× MG buffer, and 15 g of Agar (Sigma: A5306) (for solid media).

Various experiments were directed to transforming a plant part and/or PCM to produce an anthocyanin. The plant part or PCM was transformed using the plasmid vector 675 of FIG. 6K which encodes particular transcription factors. In specific experiments, Cannabis plant parts were transformed with the plasmid vector 675 to produce anthocyanin. Anthocyanin exist in wild-type tissue of different plants, however, is not always present in a root tissue. The anthocyanin pathway is activated in the PCM culture via overexpression of the particular transcription factors, the MYB transcription factors.

An example bio-pathway for production of anthocyanin, consistent with the present disclosure, is illustrated by Mekapogu, M. etal. Anthocyanins in Floral Colors: Biosynthesis and Regulation in Chrysanthemum Flowers. Int. J. Mol. Sci. 2020, 21, 6537, which is incorporated herein by reference in its entirety for its teaching.

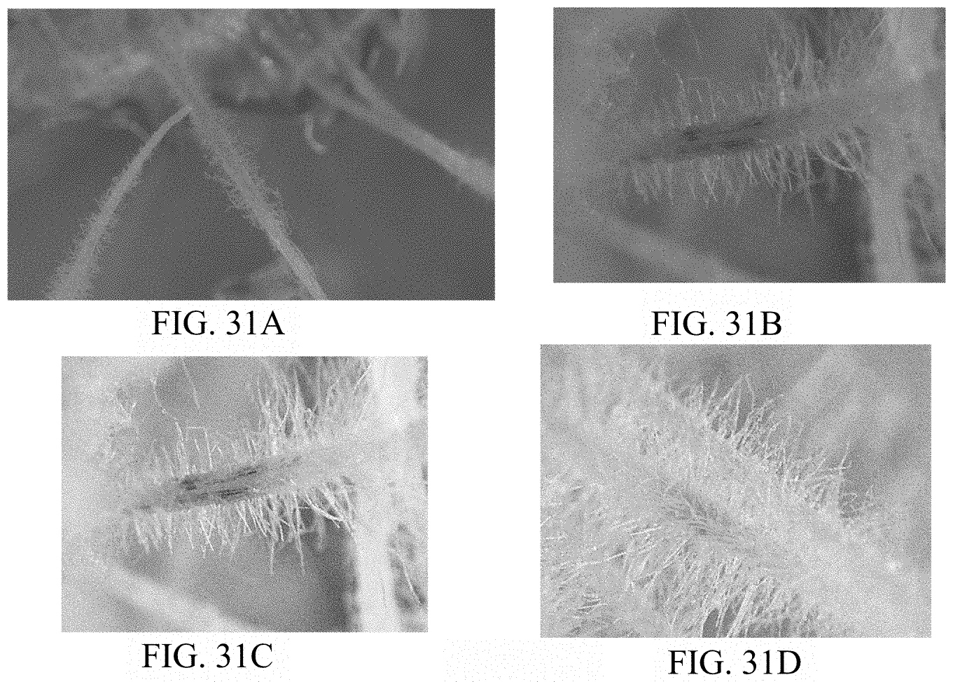

FIGS. 31A-31D illustrate example images of PCM cultures transformed to overexpress MYB transcription factors and produce anthocyanin, consistent with the present disclosure. FIGS. 31C-31D illustrate images which are enhanced with digital filters.

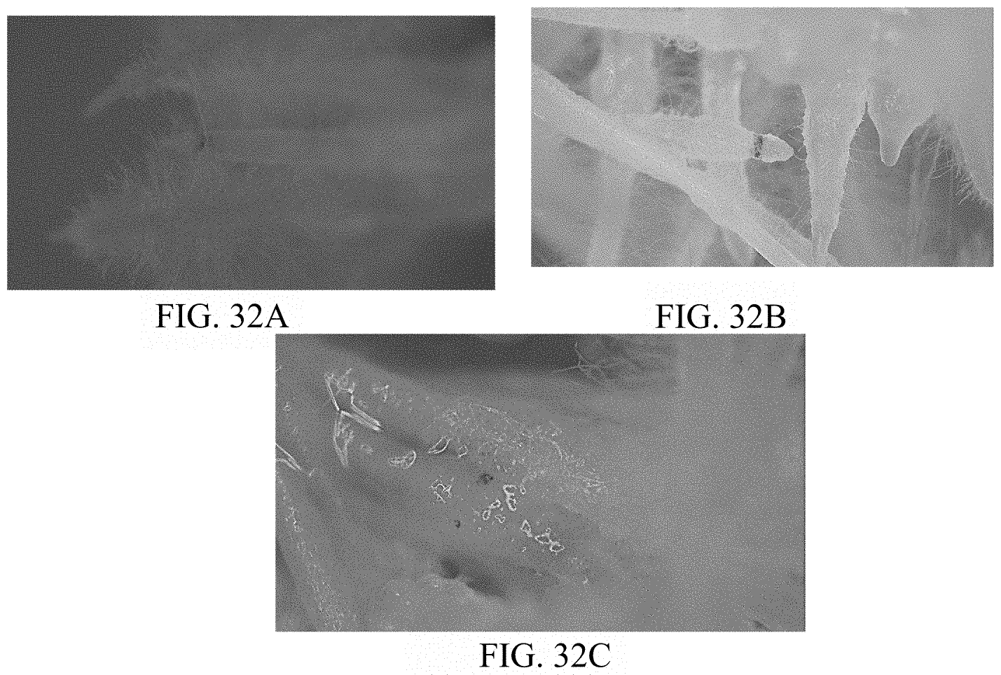

FIGS. 32A-32C illustrate example images of PCM cultures transformed to overexpress MYB transcription factors and produce anthocyanin, consistent with the present disclosure. The images of FIGS. 32A-32C are digitally altered to more clearly illustrate the different colors.

Various experimental embodiments were directed to assessing different growth conditions and resulting growth rates of PCM tissue, as well as assessing growth rates over wild-types of tissues. The different growth conditions included assessing the above-listed culture mediums including liquid forms, solid forms, different basal salts, different sugar amounts, and pH buffers. In various embodiments, different light/dark conditions were assessed. In some experimental embodiments, culture mediums that were liquid-based and included DKW performed better than those containing WPM or MS.

Some experimental embodiments were directed to using different plant clones to generate PCMs and selecting the optimal PCM from the plurality of clones based on increases in biomass while culturing under growth conditions. The plant clones were transformed with an A4 bacterium strain containing the Ri plasmids and placed in flasks. The weight gain was tracked over a period of around one month. Such experiments illustrated the genetic variability between clones. Table 2 illustrates different example clone results from the experiments. Additional clones were tested.

TABLE 2

| | Percent Weight Increase |
|---|---|
| Clone | After About One Month |
| PCM11-11 | 4195.04% |
| PCM11-6 | 4310.22% |
| PCM11-10 | 5587% |
| PCM11-23 | 6787.95% |
| PCM04-2-1 | 5852.82% |
| PCM16-7 | 806.99% |
| PCM16-5 | 835.57% |
| PCM05-2-1 | 730.95% |
| PCM05-2-8 | 969.09% |
| PCM05-1-16 | 759.7% |

Various experiments were directed to assessing growth rates and increases in growth rates of PCM tissue in PCM cultures as compared to wild-type roots grown in the field and via aeroponics. For assessing growth rates of wild-type roots, the calculation was based on grams of dried wild-type root per meter squared per month (g/m²/month). For assessing growth rates of PCM tissue, the calculation was based on dried PCM tissue g/m²/month. In various experimental embodiments and using calculations described above, it was estimated that wild-type plants grown in a field produce about 6-7 root g/m²/month and grown using aeroponics produce about 13 root g/m²/month. In contrast, PCM cultures produced PCM tissue at about 120-190 PCM g/m²/month, which was about 9-14 fold increase in root tissue production over production of wild-type roots grown using aeroponics and about 18-28 fold increase in root tissue production over wild-type roots grown in the field. Further increases in growth can be shown via additional optimization. Tables 3-5 illustrate example mass and growth rate calculations.

TABLE 3
Root Tissue Biomass

| Growth Environment | Biomass |
|---|---|
| Grown in Field | 6-7 root g/m²/month |
| | (estimates using various literature) |
| Aeroponics | 13.2 root g/m²/month |
| PCM | 120-190 PCM g/m²/month |

TABLE 4
Aeroponics Growth Calculations

| Amount | Unit |
|---|---|
| 0.4 | Tray area (m²) |
| 96 | Plants per tray |
| 240 | plants/m² |
| 55.4 | average dry weight roots |
| | per plant (mg) |
| 13000 | mg/m²/month |
| 13.2 | g/m²/month |

TABLE 5
PCM Growth Calculations

| Measurement | Amount | Unit |
|---|---|---|
| PCM mass gained weight | 0.08 | kg/m²/month |
| Time grown (PCM) | 29 | Days |
| Growth space area | 0.0208 | m² |
| Dry weight mass correction | 5 | percent |
| PCM dry weight growth | 0.192 = (0.08 × 5%) | kg/m²/month |
| per area per month | /0.0208 | |

Embodiments are not limited to the transformations illustrated by the experimental embodiments and can be directed to variety of different transformations and PCM generations in a variety of different plant species to achieve different growth rates and/or biomass of PCM tissue. Different PCM-derived compounds can be produced by the PCMs, as described throughout.

1. A method comprising:
contacting a plant part with a nucleotide sequence encoding a gene that induces plant cell matrix (PCM) formation, the PCM comprising plant cells of a plurality of differentiated plant cell types that are transformed by the contact with the nucleotide sequence; and
culturing the plant part under growth conditions to enhance the PCM formation.

2. The method of claim 1, wherein the nucleotide sequence comprises a root-inducing (Ri) plasmid or a tumor-inducing (Ti) plasmid and encodes the gene that induces the PCM formation, and the plurality of differentiated plant cell types comprise cells selected from:
plant stem cells, maturing cells, mature cells, and a combination thereof.

3. The method of claim 1, wherein the growth conditions comprise conditions selected from:
a liquid culture medium, a type of culture medium, an amount of contact with the culture medium, a type of contact with the culture medium, a plant type, and a combination thereof.

4. The method of claim 1, wherein the plant part is a seedling, a petiole, an internode, a node, a meristem, or a leaf.

5. The method of claim 1, wherein the plant part is from a Cannabaceae plant, a Brassicaceae plant, a Solanaceae plant, a Fabaceae plant, or an Apiacea plant.

6. The method of claim 1, wherein culturing the plant part under the growth conditions comprises intermittently contacting the plant part with a culture medium containing sugar and basal salt.

7. The method of claim 6, wherein intermittently contacting the plant part with the culture medium comprises cycling between contacting the plant part with the culture medium and not contacting the plant part with the culture medium at a duty cycle of between about 1 percent and about 25 percent.

8. The method of claim 6, wherein the culture medium comprises a liquid culture medium and the basal salt comprises a Driver and Kuniyuki Walnut (DKW) basal salt.

9. The method of claim 1, wherein culturing of the plant part is performed under the growth conditions to enhance the PCM formation, thereby resulting in production of PCM tissue at a greater production level than tissue produced by a wild-type plant or a plant grown in a field.

10. The method of claim 9, wherein the production of PCM tissue in the PCM is at least about 2-fold to about 500-fold a production level as compared to production of the tissue in wild-type plant or plant grown in a field.

11. The method of claim 1, wherein contacting the plant part with the nucleotide sequence and culturing the plant part comprises:
contacting the plant part with a bacterium strain comprising a root-inducing (Ri) plasmid or a tumor-inducing (Ti) plasmid and the nucleotide sequence encoding the gene that induces the PCM formation; and
culturing the plant part to enhance transformation and induce the PCM formation by at least 2-fold a production level as compared to tissue production of a wild-type plant.

12. The method of claim 1, wherein contacting the plant part with the nucleotide sequence and culturing the plant part comprises:
contacting the plant part with a root-inducing (Ri) plasmid to transform plant cells of the plant part using particle bombardment mediated transformation, microinjection, liposome injection, polyethylene glycol (PEG) delivery to protoplasts, or agroinfiltration.

13. A method comprising:
contacting a plant part with a nucleotide sequence encoding a gene that induces plant cell matrix (PCM) formation, the PCM comprising plant cells of a plurality of differentiated plant cell types that are transformed by the contact with the nucleotide sequence; and
culturing the plant part to enhance PCM formation, thereby resulting in production of PCM tissue by the PCM at a greater production level than root tissue produced by a wild-type plant or a plant grown in a field.

14. The method of claim 13, wherein the production of the PCM tissue by the PCM is at least about 2-fold to about 500-fold a production level of the root tissue produced by the wild-type plant or the plant grown in the field.

15. The method of claim 13, wherein:
the nucleotide sequence comprises a root-inducing (Ri) plasmid or a tumor-inducing (Ti) plasmid and encodes the gene that induces PCM formation; and
the PCM comprises the plant cells transformed by the nucleotide sequence to express the gene that induces PCM formation and the PCM tissue exhibiting a PCM phenotype formed from the transformed plant cells.

16. The method of claim 13, wherein culturing comprises culturing the plant part under growth conditions to enhance transformation and induce the PCM formation by at least 2-fold a production level as compared to the root tissue produced by the wild-type plant or the plant grown in the field.

17. The method of claim 16, wherein the growth conditions comprise conditions selected from:
a liquid culture medium, a type of culture medium, an amount of contact with the culture medium, a type of contact with the culture medium, a plant type, and a combination thereof.

18. The method of claim 16, wherein the growth conditions comprise intermittently contacting the plant part with a liquid culture medium containing sugar and basal salt by cycling between contact of the plant part with the liquid culture medium and no contact of the plant part with the liquid culture medium.

19. The method of claim 13, wherein culturing the plant part induces production of a PCM-derived compound and the PCM-derived compound comprises at least one of:
a core precursor compound that is produced by the PCM at an increased production level as compared to production in the root tissue of the wild-type plant; and
a recombinant compound.

20. A plant cell matrix (PCM) culture comprising a plurality of plant cells of differentiated plant cell types that express a gene that induces PCM formation, and plant tissue exhibiting a PCM phenotype formed from the plurality of plant cells.


SEQUENCE LISTING (95 sequences)

SEQ ID NO: 1 - DNA, 11558 bp, Artificial sequence
  Synthetic
ggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgattaaagtgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcgaaccctaggttaaagctcatcatgcttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagccgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcacgaattcggccgaggataatgataggagaagtgaaaagatgagaaagagaaaaagattagtcttcatggtggtgtgcgatcgcgagaaatattggttagtatctgatgatccttcaaatgggaatgaatgccttcttatatagagggaattcttttgtggtcgtcactgcgttcgtcatacgcattagtgagtgggctgtcaggacagctcttttccacgttattttgttccccacttgtactagaggaatctgctttatctttgcaataaaggcaaagatgcttttggtaggtgcgcctaacaattctgcaccattccttttttgtctggtccccacaagccagctgctcgatgttgacaagattactttcaaagatgcccactaactttaagtcttcggtggatgtctttttctgaaacttactgaccatgatgcatgtgctggaacagtagtttactttgattgaagattcttcattgatctcctgtagcttttggctaatggtttggagactctgtaccctgaccttgttgaggctttggactgagaattcttccttacaaacctttgaggatgggagttccttcttggttttggcgataccaatttgaataaagtgatatggctcgtaccttgttgattgaacccaatctggaatgctgctaaatattttgatgacctgcaggcgcgaatgcatctaaggtgtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcctcagcacaccaccatggcacagattaacaacatggctcaaggcatccagacacttaatcccaattccaacttccataagccccaagtacctaaaagctcaagttttcttgtttttggatctaaaaagctgaaaaattcagcaaattctatgttggttttgaaaaaggattcaatttttatgcaaaagttttgttcctttaggatttcagcatcagtggctacagcctgcatggaggatcttcctgaaaacttggatcaggagtctcttttccaaggattgagagagttcggaatctctacaacttctgcttcttacgctcctcttggtttcggagattaccattggcatatcacaggagatgatggacaaagatggttcgctacagtttctgatttggagcataaggaacattgtggacatggtgctcctgctgctttgagaggtcttagaagagctatggatacagctgttcatttgagagagcaaggaggtcttcctttcgttgttgctcctagaactacatctgatggagcttctttggttcctcttgattctagatacgctttgactgttttcccacatgtttctgctagaccaggagagttcggtcagaaacttacagaaagagagagagatcaagttcttgttcttttggctgagcttcatggacaagctcctcctaagtgtactcctacaactgatatggttccaactggtcttgatggagttcatactgctcttgctgaaccttctggtacttggacaggtggaccattctctgaacctgctagagaacttttggctgaacatgaggctactttgagaggaagaatggctgagttcggtgaattggttgctagagttagaggaagaggtgctcctttggttgttactcatggagagccacatcctggaaatcttatcttgggagaggatggttatgttttggttgattgggatactgttggtcttgctatcccagaaagagatctttctttgatttctgatgatcctgctgctcttgctagatacacagaacttactggacatacacctgatccagctgctttggctctttacagattgagatggtctttgcttgatgttgctgagtttgttgaatggttcagaggagaacatcaaagaacttctgatactgaggctgcttggcagtctttcgctgagactcttgatcatcttaactctgaagttccttcttaatgactagtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagcgcggccgcgagctcgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgc

SEQ ID NO: 2 - DNA, 795 bp, Artificial Sequence
  Synthetic
ttaaagctcatcatgcttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagccgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcacgaattcggccgaggataatgataggagaagtgaaaagatgagaaagagaaaaagattagtcttcat

SEQ ID NO: 3 - DNA, 977 bp, Artificial sequence
  Synthetic
ggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttag

SEQ ID NO: 4 - DNA, 597 bp, Artificial Sequence
  Synthetic
gagaaatattggttagtatctgatgatccttcaaatgggaatgaatgccttcttatatagagggaattcttttgtggtcgtcactgcgttcgtcatacgcattagtgagtgggctgtcaggacagctcttttccacgttattttgttccccacttgtactagaggaatctgctttatctttgcaataaaggcaaagatgcttttggtaggtgcgcctaacaattctgcaccattccttttttgtctggtccccacaagccagctgctcgatgttgacaagattactttcaaagatgcccactaactttaagtcttcggtggatgtctttttctgaaacttactgaccatgatgcatgtgctggaacagtagtttactttgattgaagattcttcattgatctcctgtagcttttggctaatggtttggagactctgtaccctgaccttgttgaggctttggactgagaattcttccttacaaacctttgaggatgggagttccttcttggttttggcgataccaatttgaataaagtgatatggctcgtaccttgttgattgaacccaatctggaatgctgctaaatattttgatga

SEQ ID NO: 5 - DNA, 1815 bp, Artificial Sequence
  Synthetic
gtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggt

SEQ ID NO: 6 - DNA, 996 bp, Artificial Sequence
  Synthetic
atggaggatcttcctgaaaacttggatcaggagtctcttttccaaggattgagagagttcggaatctctacaacttctgcttcttacgctcctcttggtttcggagattaccattggcatatcacaggagatgatggacaaagatggttcgctacagtttctgatttggagcataaggaacattgtggacatggtgctcctgctgctttgagaggtcttagaagagctatggatacagctgttcatttgagagagcaaggaggtcttcctttcgttgttgctcctagaactacatctgatggagcttctttggttcctcttgattctagatacgctttgactgttttcccacatgtttctgctagaccaggagagttcggtcagaaacttacagaaagagagagagatcaagttcttgttcttttggctgagcttcatggacaagctcctcctaagtgtactcctacaactgatatggttccaactggtcttgatggagttcatactgctcttgctgaaccttctggtacttggacaggtggaccattctctgaacctgctagagaacttttggctgaacatgaggctactttgagaggaagaatggctgagttcggtgaattggttgctagagttagaggaagaggtgctcctttggttgttactcatggagagccacatcctggaaatcttatcttgggagaggatggttatgttttggttgattgggatactgttggtcttgctatcccagaaagagatctttctttgatttctgatgatcctgctgctcttgctagatacacagaacttactggacatacacctgatccagctgctttggctctttacagattgagatggtctttgcttgatgttgctgagtttgttgaatggttcagaggagaacatcaaagaacttctgatactgaggctgcttggcagtctttcgctgagactcttgatcatcttaactctgaagttccttcttaatga

SEQ ID NO: 7 - DNA, 26 bp, Artificial Sequence
  Synthetic
tgtttacaccacaatatatcctgcca

SEQ ID NO: 8 - DNA, 24 bp, Artificial sequence
  Synthetic
tttacccgccaatatatcctgtca

SEQ ID NO: 9 - DNA, 276 bp, Artificial Sequence
  Synthetic
tcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 10 - DNA, 297 bp, Artificial Sequence
  Synthetic
agtgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcgaac

SEQ ID NO: 11 - DNA, 10486 bp, Artificial Sequence
  Synthetic
ggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcgcagtgccaagcttacgacgtcggaatcgagattgcctcggtacaagagtaatcgacctgcagactggctgtgtataagggagcctgacatgcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggaggacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcacggatcgcccttcccaacagttgcgcagcctgaatggcgaatgggagagatcggaatcgagattgcctcggtacaaggaatctcaattctgagatttttcaaatcctgcaggcgcgaatgcatctaaggtgtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcctcagcgtatttttacaacaattaccaacaacaacaaacaacaaacaacattacaattactatttacaattacaccatgggtcatcaccatcaccatcacaactcttcatctaacaataacaataacaataacaataacaatcttggtattgagggaaggggttctattggagctgcatctatggagttctgtttcgatgtttttaaggagttgaaggtgcatcacgcaaacgaaaacattttctattgcccaatcgctattatgtctgctcttgcaatggtttacttgggtgcaaaggattcaactagaacacaaattaacaaggttgtgaggtttgataagcttccaggattcggagattctattgaagctcaatgtggaacctcagttaacgtgcattcatctcttagagatatcttgaaccagatcactaagcctaacgatgtttactctttttcacttgcttctagattgtatgcagaggaaaggtacccaattcttcctgagtatttgcaatgcgtgaaggaactttacagaggtggacttgagccaattaacttccaaacagctgcagatcaggctagggaacttattaattcttgggttgagtcacagaccaacggtattattagaaacgttttgcaaccttcatctgtggattctcagactgctatggttcttgtgaatgcaattgtgtttaagggattgtgggagaagacattcaaggatgaagatacccaagcaatgccattcagagttacagagcaggaatcaaagcctgtgcaaatgatgtaccagattggtctttttagagttgcttctatggcatcagagaagatgaagattcttgaattgccattcgcttctggtaccatgtcaatgttggttcttttgcctgatgaggtgtctggacttgagcaattggaatcaattattaacttcgagaagcttaccgagtggacttcttctaacgttatggaggaaagaaagattaaggtgtatttgcctaggatgaagatggaggaaaagtacaaccttacttctgttttgatggcaatgggtattacagatgtgttctcatcttcagctaatctttcaggaatttcttcagcagaatctttgaagatttcacaggctgttcatgctgcacacgctgagattaacgaagcaggaagggaggttgtgggttctgctgaagcaggagttgatgctgcatctgtgtcagaggaattcagagctgatcatccatttcttttctgtattaagcacattgctacaaatgcagttttgtttttcggaagatgcgtgtctccttgactagtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagcgcggccgcgagctcgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgc

SEQ ID NO: 12 - DNA, 1162 bp, Artificial Sequence
  Synthetic
atggggttctattggagctgcatctatggagttctgtttcgatgtttttaaggagttgaaggtgcatcacgcaaacgaaaacattttctattgcccaatcgctattatgtctgctcttgcaatggtttacttgggtgcaaaggattcaactagaacacaaattaacaaggttgtgaggtttgataagcttccaggattcggagattctattgaagctcaatgtggaacctcagttaacgtgcattcatctcttagagatatcttgaaccagatcactaagcctaacgatgtttactctttttcacttgcttctagattgtatgcagaggaaaggtacccaattcttcctgagtatttgcaatgcgtgaaggaactttacagaggtggacttgagccaattaacttccaaacagctgcagatcaggctagggaacttattaattcttgggttgagtcacagaccaacggtattattagaaacgttttgcaaccttcatctgtggattctcagactgctatggttcttgtgaatgcaattgtgtttaagggattgtgggagaagacattcaaggatgaagatacccaagcaatgccattcagagttacagagcaggaatcaaagcctgtgcaaatgatgtaccagattggtctttttagagttgcttctatggcatcagagaagatgaagattcttgaattgccattcgcttctggtaccatgtcaatgttggttcttttgcctgatgaggtgtctggacttgagcaattggaatcaattattaacttcgagaagcttaccgagtggacttcttctaacgttatggaggaaagaaagattaaggtgtatttgcctaggatgaagatggaggaaaagtacaaccttacttctgttttgatggcaatgggtattacagatgtgttctcatcttcagctaatctttcaggaatttcttcagcagaatctttgaagatttcacaggctgttcatgctgcacacgctgagattaacgaagcaggaagggaggttgtgggttctgctgaagcaggagttgatgctgcatctgtgtcagaggaattcagagctgatcatccatttcttttctgtattaagcacattgctacaaatgcagttttgtttttcggaagatgcgtgtctccttga

SEQ ID NO: 13 - DNA, 276 bp, Artificial Sequence
  Synthetic
atgaccatgattacgccaagcgcgcaattaaccctcactaaagggaacaaaagctggaggacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcacggatcgcccttcccaacagttgcgcagcctgaatggcgaatgg

SEQ ID NO: 14 - DNA, 122 bp, Artificial Sequence
  Synthetic
gcgcaacgcaattaatgtgagttagctcactcattaggcaccccaggctttacactttatgcttccggctcgtatgttgtgtggaattgtgagcggataacaatttcacacaggaaacagct

SEQ ID NO: 15 - DNA, 68 bp, Artificial Sequence
  Synthetic
gtatttttacaacaattaccaacaacaacaaacaacaaacaacattacaattactatttacaattaca

SEQ ID NO: 16 - DNA, 18 bp, Artificial Sequence
  Synthetic
catcaccatcaccatcac

SEQ ID NO: 17 - DNA, 47 bp, Artificial Sequence
  Synthetic
actcttcatctaacaataacaataacaataacaataacaatcttggt

SEQ ID NO: 18 - DNA, 12 bp, Artificial Sequence
  Synthetic
attgagggaagg

SEQ ID NO: 19 - DNA, 9434 bp, Artificial Sequence
  Synthetic
ggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcgcagtgccaagcttacgacgtcggaatcgagattgcctcggtacaagagtaatcgacctgcagactggctgtgtataagggagcctgacatgcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggaggacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcacggatcgcccttcccaacagttgcgcagcctgaatggcgaatgggagagatcggaatcgagattgcctcggtacaaggaatctcaattctgagatttttcaaatcctgcaggcccgagcaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgggggactctagaggatccccgatccctacagcctcagcgtatttttacaacaattaccaacaacaacaaacaacaaacaacattacaattactatttacaattacaccatgggtcatcaccatcaccatcacaactcttcatctaacaataacaataacaataacaataacaatcttggtattgagggaaggggttctattggagctgcatctatggagttctgtttcgatgtttttaaggagttgaaggtgcatcacgcaaacgaaaacattttctattgcccaatcgctattatgtctgctcttgcaatggtttacttgggtgcaaaggattcaactagaacacaaattaacaaggttgtgaggtttgataagcttccaggattcggagattctattgaagctcaatgtggaacctcagttaacgtgcattcatctcttagagatatcttgaaccagatcactaagcctaacgatgtttactctttttcacttgcttctagattgtatgcagaggaaaggtacccaattcttcctgagtatttgcaatgcgtgaaggaactttacagaggtggacttgagccaattaacttccaaacagctgcagatcaggctagggaacttattaattcttgggttgagtcacagaccaacggtattattagaaacgttttgcaaccttcatctgtggattctcagactgctatggttcttgtgaatgcaattgtgtttaagggattgtgggagaagacattcaaggatgaagatacccaagcaatgccattcagagttacagagcaggaatcaaagcctgtgcaaatgatgtaccagattggtctttttagagttgcttctatggcatcagagaagatgaagattcttgaattgccattcgcttctggtaccatgtcaatgttggttcttttgcctgatgaggtgtctggacttgagcaattggaatcaattattaacttcgagaagcttaccgagtggacttcttctaacgttatggaggaaagaaagattaaggtgtatttgcctaggatgaagatggaggaaaagtacaaccttacttctgttttgatggcaatgggtattacagatgtgttctcatcttcagctaatctttcaggaatttcttcagcagaatctttgaagatttcacaggctgttcatgctgcacacgctgagattaacgaagcaggaagggaggttgtgggttctgctgaagcaggagttgatgctgcatctgtgtcagaggaattcagagctgatcatccatttcttttctgtattaagcacattgctacaaatgcagttttgtttttcggaagatgcgtgtctccttgactagtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagcgcggccgcgagctcgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgc

SEQ ID NO: 20 - DNA, 781 bp, Artificial Sequence
  Synthetic
cccgagcaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgggggactctagaggatccccgatccctacag

SEQ ID NO: 21 - DNA, 13573 bp, Artificial Sequence
  Synthetic
gcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgagctcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcgggtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctagcttggccgggtcccgggctgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatcaaaggtagaagcagaaacttacctagcatggtggccactccaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtagaagccatcttccttttctactgtcctttcgatgaagtgacagatagctgggcaatggaatccgaggaggtttcccgatattaccctttgttgaaaagtctcaatagccctctggtcttctgagactgtatctttgatattcttggagtagacgagagtgtcgtgctccaccatgttgaccctgcaggtcatcaaaatatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactaaccaatatttctcgcgatcgcacaccaccatggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctaccaattgttgaattttgatttgttgaagttggctggagatgttgaatctaatcctggacctatgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgcccaactcctcaactttgacctgctcaaattggcaggcgatgtcgaatcgaatccggggcctatgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaacgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtgatagtgacctagggttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacactcctgcaggatttgaaaaatctcagaattgagattccttgtaccgaggcaatctcgattccgagtaatcccattcgccattcaggctgcgcaactgttgggaagggcgatccgtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgcatgtcaggctcccttatacacagccagtctgcaggtcgaatacacttgtaccgaggcaatctcgattccgacaagctaagcttggcactgcgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacc

SEQ ID NO: 22 - DNA, 4861 bp, Artificial Sequence
  Synthetic
tcatcaaaatatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactaaccaatatttctcgcgatcgcacaccaccatggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctaccaattgttgaattttgatttgttgaagttggctggagatgttgaatctaatcctggacctatgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgcccaactcctcaactttgacctgctcaaattggcaggcgatgtcgaatcgaatccggggcctatgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaacgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtgatagtgacctagggttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacact

SEQ ID NO: 23 - DNA, 597 bp, Artificial Sequence
  Synthetic
tcatcaaaatatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactaaccaatatttctc

SEQ ID NO: 24 - DNA, 1491 bp, Artificial Sequence
  Synthetic
atggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctac

SEQ ID NO: 25 - DNA, 60 bp, Artificial Sequence
  Synthetic
caattgttgaattttgatttgttgaagttggctggagatgttgaatctaatcctggacct

SEQ ID NO: 26 - DNA, 825 bp, Artificial Sequence
  Synthetic
atgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgcc

SEQ ID NO: 27 - DNA, 60 bp, Artificial Sequence
  Synthetic
caactcctcaactttgacctgctcaaattggcaggcgatgtcgaatcgaatccggggcct

SEQ ID NO: 28 - DNA, 1509 bp, Artificial Sequence
  Synthetic
atgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaacgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtgatagtga

SEQ ID NO: 29 - DNA, 297 bp, Artificial Sequence
  Synthetic
gttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacact

SEQ ID NO: 30 - DNA, 1661 bp, Artificial Sequence
  Synthetic
gtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctggagtggccaccatgctaggtaagtttctgcttctacctttgatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcagcccgggacccggccaagctagcttggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggacccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagt

SEQ ID NO: 31 - DNA, 422 bp, Artificial Sequence
  Synthetic
gtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggac

SEQ ID NO: 32 - DNA, 187 bp, Artificial Sequence
  Synthetic
gtaagtttctgcttctacctttgatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcag

SEQ ID NO: 33 - DNA, 825 bp, Artificial Sequence
  Synthetic
atgctagcccgggacccggccaagctagcttggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 34 - DNA, 160 bp, Artificial Sequence
  Synthetic
tttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagt

SEQ ID NO: 35 - DNA, 24 bp, Artificial Sequence
  Synthetic
tgacaggatatattggcgggtaaa

SEQ ID NO: 36 - DNA, 26 bp, Artificial Sequence
  Synthetic
tggcaggatatattgtggtgtaaaca

SEQ ID NO: 37 - DNA, 398 bp, Artificial Sequence
  Synthetic
gcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggaggacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcacggatcgcccttcccaacagttgcgcagcctgaatggcgaatgg

SEQ ID NO: 38 - DNA, 122 bp, Artificial Sequence; identical to SEQ ID NO: 14
  Synthetic

SEQ ID NO: 39 - DNA, 276 bp, Artificial Sequence; identical to SEQ ID NO: 13
  Synthetic

SEQ ID NO: 40 - DNA, 977 bp, Artificial Sequence; identical to SEQ ID NO: 3
  Synthetic

SEQ ID NO: 41 - DNA, 182 bp, Artificial Sequence
  Synthetic
ggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataa

SEQ ID NO: 42 - DNA, 795 bp, Artificial Sequence
  Synthetic
atggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttag

SEQ ID NO: 43 - DNA, 13585 bp, Artificial Sequence
  Synthetic
gcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgagctcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcgggtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctagcttggccgggtcccgggctgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatcaaaggtagaagcagaaacttacctagcatggtggccactccaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtagaagccatcttccttttctactgtcctttcgatgaagtgacagatagctgggcaatggaatccgaggaggtttcccgatattaccctttgttgaaaagtctcaatagccctctggtcttctgagactgtatctttgatattcttggagtagacgagagtgtcgtgctccaccatgttgaccctgcaggtcatcaaaatatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactaaccaatatttctcgcgatcgcacaccaccatggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctacggaagcggagctactaacttcagcctgctgaagcaggctggagacgtggaggagaaccctggacctatgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgccggttcaggtgccactaatttttctctcttgaaacaggccggtgacgttgaagagaacccaggtccaatgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaacgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtgatagtgacctagggttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacactcctgcaggatttgaaaaatctcagaattgagattccttgtaccgaggcaatctcgattccgagtaatcccattcgccattcaggctgcgcaactgttgggaagggcgatccgtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgcatgtcaggctcccttatacacagccagtctgcaggtcgaatacacttgtaccgaggcaatctcgattccgacaagctaagcttggcactgcgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacc

SEQ ID NO: 44 - DNA, 4873 bp, Artificial Sequence
  Synthetic
tcatcaaaatatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactaaccaatatttctcgcgatcgcacaccaccatggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctacggaagcggagctactaacttcagcctgctgaagcaggctggagacgtggaggagaaccctggacctatgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgccggttcaggtgccactaatttttctctcttgaaacaggccggtgacgttgaagagaacccaggtccaatgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaacgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtgatagtgacctagggttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacact

SEQ ID NO: 45 - DNA, 66 bp, Artificial Sequence
  Synthetic
ggaagcggagctactaacttcagcctgctgaagcaggctggagacgtggaggagaaccctggacct

SEQ ID NO: 46 - DNA, 66 bp, Artificial Sequence
  Synthetic
ggttcaggtgccactaatttttctctcttgaaacaggccggtgacgttgaagagaacccaggtcca

SEQ ID NO: 47 - DNA, 15647 bp, Artificial Sequence
  Synthetic
gcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgagctcgcggccgcgctatgaccatgattacgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgagagctcattacgccgcttttgtgtcagagagttcaatcttagctccgagacccgccattaaatccatgaactcggggaaactggttgctatcatagtagcgtcatcaactgtaaccggattctcagaaacaagtcccattaccaagaaggacatagctatacggtgatcgagatgcgtagcaaccgcagccccggatgcgtttccaagccccttaccatctggacgtcctctaacgaccaagctggtttcgccttcgtcgcaatctacaccgttaagcttcagcccgttagcaaccgcggaaagtctgtcggactccttcaccctcaactcttcgagcccgttcatgacggtagctccttcagcaaaggcagcagcgacggccagtattgggtattcatcaatcatagatggagcgcggtcttctgggacagtcacgcctttaagagtagagcttctaactcgtaggtcagcgacatcctcaccaccggctagtcttggattaatcacttcaatgtcagcgcccatctcttgtagtgtcaggattagcccagtccgtgtagggttcatgagcacgttcaagatggtgacatcagagccgggcaccaacagcgcagcaacgagagggaaggcagtactgctagggtcgccggggacgtcgattacttggccagttaactttccgcgtccttccaacctaattgtccgcacgccgtcagcatctgtctcaactgtcagattggctccgaagccttgcagcattttctcagtatgatcccttgtcatgataggttcaatcacggtcgttatcccaggtgtgttcaatccagcgagcaacactgcgctcttgacttgcgcactagccataggcacgcggtaagtgattggtgtaggtgtcttaggtccccgaagggtgacaggcaatctatctccatcctctgacttcacttgaacacccatctccctcaatgggttaagaactcttcccatgggcctttttgtcaaagaagcatctcctataaatgtactatcgaaatcgtacacgcctacaagccccatggtgaggcggcatccggtggctgcatttccgaaatccaacggagcttcaggtgcaagcaggcctccatttccaactccatcaattatccaagtgtctccctcctttctaatcctcgcccccatggcttgcatagcttttccggtattgatcacatcttctccttcgagcagtcccgtgattcgggtctctccactagccaatccaccaaacatgaaggatctatgagaaatacttttgtcccccgggattctcacagtgccactaagtccgctagattttctcgcagtggcaggccttgaggacgctccgtgcgacatgcaggctgtagccactgatgctgaaatcctaaaggaacaaaacttttgcataaaaattgaatcctttttcaaaaccaacatagaatttgctgaatttttcagctttttagatccaaaaacaagaaaacttgagcttttaggtacttggggcttatggaagttggaattgggattaagtgtctggatgccttgagccatgttgttaatctgtgccatggtggtgtgctgaggacctgcaaagttgaaaaccacaaacgaatcagaatgatgaaatttaacagctatccgactgaattaagtacatctcaagaacagatctgattaatttcagggaaaaaaatacataatcttcggtcaggattaaccaaacaaggcggttgttaatggatctgaattttttttttctttaaaataagaagactgttaacggatctgaaaattttcttaaaaagaaaacaattaacgtaataaagaactacagagaaaaattcaagacgtgttattgtaaaagagaaagaaaagattctgacgatcgatgaaactgtacaaaccctatcttaaactcaacaagctaatcgaaaacatgatcattcacgggactaatgaataacatagacaaaataaaatagacgaaccttgaggggagaggtagggaactgcgcaaagagaatgaagtagtttgttttgaattgaactgtgaagagagacagagggtgtgggcctttttatagctaaaggggtggaatcctctccaatcacattgtgacgcgtgtccttgtgtccctatttggtgcttattacacgggtgatatgtggtgtctgatgcgttttcggagtcctttcttggaaattcaggattgcaatttgcggggtggttagcgggttggtaatgttgacggagtcagacgcgcctacgtcagcttaatctgttacaacggcgttattaaattggaccccacttcacaatttgggccggaaccactgcaagtggagcgttctcagttttgtatgggcccctctatccactacaccaacggttggctcctattttacatgattaagaaatctattgcaaagctgtgatgtttgattttttaatggactaatgaatgattggaaagctttatcatttatttttttaataaggaaatgtcttgtaatcatccacgctttaactatttaaacaaatcatgaaacttaaaggtgacggatttaccaataaaataagggtataggacagcttaattgaaaaaacaaaaatgatatagtaaagaaaacttaagagcgatgggcatccttatcatctaatatgaataagctacaccatctgatctgcagtggacaagttgtcagattatacagttgttgatggctttaagaagcataattgtattattaatatttttatatataagtgcagctctattgtttatactactaatattctcgttttgcgttttatttaataattaattgtatgtactttataaaagaaaaatatcataaaatgagaaaaaaaaacaaaaattgactgttatatttgagtgtttttttacttatatgtattatttatatgtattatgctttaatataatatttttattttactaatatagacatatcaacgcgtgtttcaaactgattttttccctctcatctagatggtgttaatgttccttttatcatcaattaaacttgttgagaaacttatttgttatattaggtatgcaatataatattcgtataaattaaaaaacaaaaaattttgagagttgtttttaaaaattgatatatgggtgagtattaaagtttaaatatgtttaagaaatcattctaagatagaatatgcgcgatgaataaataaaatacctattttttaaaaatatgatatgctattttgtagaatacataaaagatgttttgtatgcgctaacaaaaagggtttttgggaaacaaaacacatataatattatttaagatgtaaaaagtttttccagtttttttttttcaaaacataatacataattcattgatcaatataattacaccttagatgcattcgcgcctgcaggtcatcaaaatatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactaaccaatatttctcgcgatcgcacaccaccatggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctacggaagcggagctactaacttcagcctgctgaagcaggctggagacgtggaggagaaccctggacctatgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgccggttcaggtgccactaatttttctctcttgaaacaggccggtgacgttgaagagaacccaggtccaatgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaacgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtgatagtgacctagggttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacactcctgcaggatttgaaaaatctcagaattgagattccttgtaccgaggcaatctcgattccgagtaatcccattcgccattcaggctgcgcaactgttgggaagggcgatccgtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgcatgtcaggctcccttatacacagccagtctgcaggtcgaatacacttgtaccgaggcaatctcgattccgacaagctaagcttggcactgcgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacc

SEQ ID NO: 48 - DNA, 4861 bp, Artificial Sequence; identical to SEQ ID NO: 22
  Synthetic

SEQ ID NO: 49 - DNA, 3697 bp, Artificial Sequence
  Synthetic
gtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcctcagcacaccaccatggcacagattaacaacatggctcaaggcatccagacacttaatcccaattccaacttccataagccccaagtacctaaaagctcaagttttcttgtttttggatctaaaaagctgaaaaattcagcaaattctatgttggttttgaaaaaggattcaatttttatgcaaaagttttgttcctttaggatttcagcatcagtggctacagcctgcatgtcgcacggagcgtcctcaaggcctgccactgcgagaaaatctagcggacttagtggcactgtgagaatcccgggggacaaaagtatttctcatagatccttcatgtttggtggattggctagtggagagacccgaatcacgggactgctcgaaggagaagatgtgatcaataccggaaaagctatgcaagccatgggggcgaggattagaaaggagggagacacttggataattgatggagttggaaatggaggcctgcttgcacctgaagctccgttggatttcggaaatgcagccaccggatgccgcctcaccatggggcttgtaggcgtgtacgatttcgatagtacatttataggagatgcttctttgacaaaaaggcccatgggaagagttcttaacccattgagggagatgggtgttcaagtgaagtcagaggatggagatagattgcctgtcacccttcggggacctaagacacctacaccaatcacttaccgcgtgcctatggctagtgcgcaagtcaagagcgcagtgttgctcgctggattgaacacacctgggataacgaccgtgattgaacctatcatgacaagggatcatactgagaaaatgctgcaaggcttcggagccaatctgacagttgagacagatgctgacggcgtgcggacaattaggttggaaggacgcggaaagttaactggccaagtaatcgacgtccccggcgaccctagcagtactgccttccctctcgttgctgcgctgttggtgcccggctctgatgtcaccatcttgaacgtgctcatgaaccctacacggactgggctaatcctgacactacaagagatgggcgctgacattgaagtgattaatccaagactagccggtggtgaggatgtcgctgacctacgagttagaagctctactcttaaaggcgtgactgtcccagaagaccgcgctccatctatgattgatgaatacccaatactggccgtcgctgctgcctttgctgaaggagctaccgtcatgaacgggctcgaagagttgagggtgaaggagtccgacagactttccgcggttgctaacgggctgaagcttaacggtgtagattgcgacgaaggcgaaaccagcttggtcgttagaggacgtccagatggtaaggggcttggaaacgcatccggggctgcggttgctacgcatctcgatcaccgtatagctatgtccttcttggtaatgggacttgtttctgagaatccggttacagttgatgacgctactatgatagcaaccagtttccccgagttcatggatttaatggcgggtctcggagctaagattgaactctctgacacaaaagcggcgtaatgagctctcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 50 - DNA, 1815 bp, Artificial Sequence; identical to SEQ ID NO: 5
  Synthetic

SEQ ID NO: 51 - DNA, 216 bp, Artificial Sequence
  Synthetic
atggcacagattaacaacatggctcaaggcatccagacacttaatcccaattccaacttccataagccccaagtacctaaaagctcaagttttcttgtttttggatctaaaaagctgaaaaattcagcaaattctatgttggttttgaaaaaggattcaatttttatgcaaaagttttgttcctttaggatttcagcatcagtggctacagcctgc

SEQ ID NO: 52 - DNA, 1371 bp, Artificial Sequence
  Synthetic
atgtcgcacggagcgtcctcaaggcctgccactgcgagaaaatctagcggacttagtggcactgtgagaatcccgggggacaaaagtatttctcatagatccttcatgtttggtggattggctagtggagagacccgaatcacgggactgctcgaaggagaagatgtgatcaataccggaaaagctatgcaagccatgggggcgaggattagaaaggagggagacacttggataattgatggagttggaaatggaggcctgcttgcacctgaagctccgttggatttcggaaatgcagccaccggatgccgcctcaccatggggcttgtaggcgtgtacgatttcgatagtacatttataggagatgcttctttgacaaaaaggcccatgggaagagttcttaacccattgagggagatgggtgttcaagtgaagtcagaggatggagatagattgcctgtcacccttcggggacctaagacacctacaccaatcacttaccgcgtgcctatggctagtgcgcaagtcaagagcgcagtgttgctcgctggattgaacacacctgggataacgaccgtgattgaacctatcatgacaagggatcatactgagaaaatgctgcaaggcttcggagccaatctgacagttgagacagatgctgacggcgtgcggacaattaggttggaaggacgcggaaagttaactggccaagtaatcgacgtccccggcgaccctagcagtactgccttccctctcgttgctgcgctgttggtgcccggctctgatgtcaccatcttgaacgtgctcatgaaccctacacggactgggctaatcctgacactacaagagatgggcgctgacattgaagtgattaatccaagactagccggtggtgaggatgtcgctgacctacgagttagaagctctactcttaaaggcgtgactgtcccagaagaccgcgctccatctatgattgatgaatacccaatactggccgtcgctgctgcctttgctgaaggagctaccgtcatgaacgggctcgaagagttgagggtgaaggagtccgacagactttccgcggttgctaacgggctgaagcttaacggtgtagattgcgacgaaggcgaaaccagcttggtcgttagaggacgtccagatggtaaggggcttggaaacgcatccggggctgcggttgctacgcatctcgatcaccgtatagctatgtccttcttggtaatgggacttgtttctgagaatccggttacagttgatgacgctactatgatagcaaccagtttccccgagttcatggatttaatggcgggtctcggagctaagattgaactctctgacacaaaagcggcgtaatga

SEQ ID NO: 53 - DNA, 280 bp, Artificial Sequence
  Synthetic
tcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcgcgcggtgtcatctatgttactagatcgggaattcgtaatcatggtcatagc

SEQ ID NO: 54 - DNA, 12024 bp, Artificial Sequence
  Synthetic
gcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgagctcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcgggtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctagcttggccgggtcccgggctgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatcaaaggtagaagcagaaacttacctagcatggtggccactccaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtagaagccatcttccttttctactgtcctttcgatgaagtgacagatagctgggcaatggaatccgaggaggtttcccgatattaccctttgttgaaaagtctcaatagccctctggtcttctgagactgtatctttgatattcttggagtagacgagagtgtcgtgctccaccatgttgaccctgcaggtcatcaaaatatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactaaccaatatttctcgcgatcgcacaccaccatggataacgcaacacttgctgtgatcctttccattttgtttgtgttttaccacattttcaaatcctttttcaccaattcttcatctcgtaggcttcctcctggtcccaaacccgtgccaatttttggcaacattttcgatcttggcgaaaagcctcatcgatcttttgccaatctatctaaaattcacggccctttgattagcctaaagttaggaagtgtaacaactattgttgtttcctcggcctctgtggccgaggaaatgttccttaaaaatgaccaagcacttgctaaccgaaccattcctgactcggttagggctggtgaccacgacaaattatccatgtcgtggttgcctgtttcccaaaaatggagaaatatgagaaaaatctccgctgtccaattactctccaaccaaaaacttgatgctagtcaacctcttagacaagctaaggtgaaacaacttttatcatacgtacaagtttgttccgaaaaaatgcaacccgtcgatattggacgggccgcatttacaacgtcacttaatttattatcaaacacatttttctcaatcgaattagcaagtcatgaatctagtgcttcccaagagtttaaacaactcatgtggaatattatggaggaaattggaaggcctaattatgctgattttttccctattcttggttacattgatccctttggtataagacgtcgtttggctggttactttgataaactcattgatgttttccaagacattattcgtgaaagacaaaagcttcgatcttctaattcttccggcgcaaaacaaacaaatgacattcttgatactcttcttaaactccatgaagataatgagttgagtatgcctgaaattaatcaccttctcgtggatatctttgacgccggaacagacacaacagcaagcacattagaatgggcgatggccgaacttgtgaaaaacccggaaatgatgactaaagttcaaattgaaatcgaacaagctcttggaaaagattgcttagacatacaagaatccgacatctcaaaactaccttatttacaagccattataaaagaaacgttacgtttacaccctcctactgtgtttttgctgcctcgaaaggcagacaatgacgtagagttatatggctacgttgtaccaaagaatgctcaagtccttgtcaatctttgggcaattggtcgtgatccaaaggtatggaaaaatccggaagtattttctcctgaaaggtttttagattgcaatatcgattataaaggacgagatttcgaacttttaccctttggtgctggtagaaggatatgccctggacttactttggcatatagaatgttgaacttgatgttggctactcttcttcaaaactacaattggaaacttgaagatggtatcaatcctaaggatttagacatggatgagaaatttgggattacattgcaaaaggttaaacctcttcaagttattccagttcccagaaacggaagcggagctactaacttcagcctgctgaagcaggctggagacgtggaggagaaccctggacctatgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgcctgatagtgactagggttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacactcctgcaggatttgaaaaatctcagaattgagattccttgtaccgaggcaatctcgattccgagtaatcccattcgccattcaggctgcgcaactgttgggaagggcgatccgtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgcatgtcaggctcccttatacacagccagtctgcaggtcgaatacacttgtaccgaggcaatctcgattccgacaagctaagcttggcactgcgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacc

SEQ ID NO: 55 - DNA, 4873 bp, Artificial Sequence; identical to SEQ ID NO: 44
  Synthetic

SEQ ID NO: 56 - DNA, 1497 bp, Artificial Sequence
  Synthetic
atggataacgcaacacttgctgtgatcctttccattttgtttgtgttttaccacattttcaaatcctttttcaccaattcttcatctcgtaggcttcctcctggtcccaaacccgtgccaatttttggcaacattttcgatcttggcgaaaagcctcatcgatcttttgccaatctatctaaaattcacggccctttgattagcctaaagttaggaagtgtaacaactattgttgtttcctcggcctctgtggccgaggaaatgttccttaaaaatgaccaagcacttgctaaccgaaccattcctgactcggttagggctggtgaccacgacaaattatccatgtcgtggttgcctgtttcccaaaaatggagaaatatgagaaaaatctccgctgtccaattactctccaaccaaaaacttgatgctagtcaacctcttagacaagctaaggtgaaacaacttttatcatacgtacaagtttgttccgaaaaaatgcaacccgtcgatattggacgggccgcatttacaacgtcacttaatttattatcaaacacatttttctcaatcgaattagcaagtcatgaatctagtgcttcccaagagtttaaacaactcatgtggaatattatggaggaaattggaaggcctaattatgctgattttttccctattcttggttacattgatccctttggtataagacgtcgtttggctggttactttgataaactcattgatgttttccaagacattattcgtgaaagacaaaagcttcgatcttctaattcttccggcgcaaaacaaacaaatgacattcttgatactcttcttaaactccatgaagataatgagttgagtatgcctgaaattaatcaccttctcgtggatatctttgacgccggaacagacacaacagcaagcacattagaatgggcgatggccgaacttgtgaaaaacccggaaatgatgactaaagttcaaattgaaatcgaacaagctcttggaaaagattgcttagacatacaagaatccgacatctcaaaactaccttatttacaagccattataaaagaaacgttacgtttacaccctcctactgtgtttttgctgcctcgaaaggcagacaatgacgtagagttatatggctacgttgtaccaaagaatgctcaagtccttgtcaatctttgggcaattggtcgtgatccaaaggtatggaaaaatccggaagtattttctcctgaaaggtttttagattgcaatatcgattataaaggacgagatttcgaacttttaccctttggtgctggtagaaggatatgccctggacttactttggcatatagaatgttgaacttgatgttggctactcttcttcaaaactacaattggaaacttgaagatggtatcaatcctaaggatttagacatggatgagaaatttgggattacattgcaaaaggttaaacctcttcaagttattccagttcccagaaac

SEQ ID NO: 57 - DNA, 825 bp, Artificial Sequence; identical to SEQ ID NO: 26
  Synthetic

SEQ ID NO: 58 - DNA, 12018 bp, Artificial Sequence
  Synthetic
gcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgagctcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcgggtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctagcttggccgggtcccgggctgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatcaaaggtagaagcagaaacttacctagcatggtggccactccaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtagaagccatcttccttttctactgtcctttcgatgaagtgacagatagctgggcaatggaatccgaggaggtttcccgatattaccctttgttgaaaagtctcaatagccctctggtcttctgagactgtatctttgatattcttggagtagacgagagtgtcgtgctccaccatgttgaccctgcaggtcatcaaaatatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactaaccaatatttctcgcgatcgcacaccaccatggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctacggaagcggagctactaacttcagcctgctgaagcaggctggagacgtggaggagaaccctggacctatgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgcctgatagtgactagggttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacactcctgcaggatttgaaaaatctcagaattgagattccttgtaccgaggcaatctcgattccgagtaatcccattcgccattcaggctgcgcaactgttgggaagggcgatccgtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgcatgtcaggctcccttatacacagccagtctgcaggtcgaatacacttgtaccgaggcaatctcgattccgacaagctaagcttggcactgcgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacc

SEQ ID NO: 59 - DNA, 4873 bp, Artificial Sequence; identical to SEQ ID NO: 44
  Synthetic

SEQ ID NO: 60 - DNA, 15275 bp, Artificial Sequence
  Synthetic
gcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgagctcgcggccgcgctatgaccatgattacgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcactagtcattaagaaggaacttcagagttaagatgatcaagagtctcagcgaaagactgccaagcagcctcagtatcagaagttctttgatgttctcctctgaaccattcaacaaactcagcaacatcaagcaaagaccatctcaatctgtaaagagccaaagcagctggatcaggtgtatgtccagtaagttctgtgtatctagcaagagcagcaggatcatcagaaatcaaagaaagatctctttctgggatagcaagaccaacagtatcccaatcaaccaaaacataaccatcctctcccaagataagatttccaggatgtggctctccatgagtaacaaccaaaggagcacctcttcctctaactctagcaaccaattcaccgaactcagccattcttcctctcaaagtagcctcatgttcagccaaaagttctctagcaggttcagagaatggtccacctgtccaagtaccagaaggttcagcaagagcagtatgaactccatcaagaccagttggaaccatatcagttgtaggagtacacttaggaggagcttgtccatgaagctcagccaaaagaacaagaacttgatctctctctctttctgtaagtttctgaccgaactctcctggtctagcagaaacatgtgggaaaacagtcaaagcgtatctagaatcaagaggaaccaaagaagctccatcagatgtagttctaggagcaacaacgaaaggaagacctccttgctctctcaaatgaacagctgtatccatagctcttctaagacctctcaaagcagcaggagcaccatgtccacaatgttccttatgctccaaatcagaaactgtagcgaaccatctttgtccatcatctcctgtgatatgccaatggtaatctccgaaaccaagaggagcgtaagaagcagaagttgtagagattccgaactctctcaatccttggaaaagagactcctgatccaagttttcaggaagatcctccatgcaggctgtagccactgatgctgaaatcctaaaggaacaaaacttttgcataaaaattgaatcctttttcaaaaccaacatagaatttgctgaatttttcagctttttagatccaaaaacaagaaaacttgagcttttaggtacttggggcttatggaagttggaattgggattaagtgtctggatgccttgagccatgttgttaatctgtgccatggtggtgtgctgaggacctgcaaagttgaaaaccacaaacgaatcagaatgatgaaatttaacagctatccgactgaattaagtacatctcaagaacagatctgattaatttcagggaaaaaaatacataatcttcggtcaggattaaccaaacaaggcggttgttaatggatctgaattttttttttctttaaaataagaagactgttaacggatctgaaaattttcttaaaaagaaaacaattaacgtaataaagaactacagagaaaaattcaagacgtgttattgtaaaagagaaagaaaagattctgacgatcgatgaaactgtacaaaccctatcttaaactcaacaagctaatcgaaaacatgatcattcacgggactaatgaataacatagacaaaataaaatagacgaaccttgaggggagaggtagggaactgcgcaaagagaatgaagtagtttgttttgaattgaactgtgaagagagacagagggtgtgggcctttttatagctaaaggggtggaatcctctccaatcacattgtgacgcgtgtccttgtgtccctatttggtgcttattacacgggtgatatgtggtgtctgatgcgttttcggagtcctttcttggaaattcaggattgcaatttgcggggtggttagcgggttggtaatgttgacggagtcagacgcgcctacgtcagcttaatctgttacaacggcgttattaaattggaccccacttcacaatttgggccggaaccactgcaagtggagcgttctcagttttgtatgggcccctctatccactacaccaacggttggctcctattttacatgattaagaaatctattgcaaagctgtgatgtttgattttttaatggactaatgaatgattggaaagctttatcatttatttttttaataaggaaatgtcttgtaatcatccacgctttaactatttaaacaaatcatgaaacttaaaggtgacggatttaccaataaaataagggtataggacagcttaattgaaaaaacaaaaatgatatagtaaagaaaacttaagagcgatgggcatccttatcatctaatatgaataagctacaccatctgatctgcagtggacaagttgtcagattatacagttgttgatggctttaagaagcataattgtattattaatatttttatatataagtgcagctctattgtttatactactaatattctcgttttgcgttttatttaataattaattgtatgtactttataaaagaaaaatatcataaaatgagaaaaaaaaacaaaaattgactgttatatttgagtgtttttttacttatatgtattatttatatgtattatgctttaatataatatttttattttactaatatagacatatcaacgcgtgtttcaaactgattttttccctctcatctagatggtgttaatgttccttttatcatcaattaaacttgttgagaaacttatttgttatattaggtatgcaatataatattcgtataaattaaaaaacaaaaaattttgagagttgtttttaaaaattgatatatgggtgagtattaaagtttaaatatgtttaagaaatcattctaagatagaatatgcgcgatgaataaataaaatacctattttttaaaaatatgatatgctattttgtagaatacataaaagatgttttgtatgcgctaacaaaaagggtttttgggaaacaaaacacatataatattatttaagatgtaaaaagtttttccagtttttttttttcaaaacataatacataattcattgatcaatataattacaccttagatgcattcgcgcctgcaggtcatcaaaatatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactaaccaatatttctcgcgatcgcacaccaccatggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctacggaagcggagctactaacttcagcctgctgaagcaggctggagacgtggaggagaaccctggacctatgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgccggttcaggtgccactaatttttctctcttgaaacaggccggtgacgttgaagagaacccaggtccaatgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaacgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtgatagtgacctagggttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacactcctgcaggatttgaaaaatctcagaattgagattccttgtaccgaggcaatctcgattccgagtaatcccattcgccattcaggctgcgcaactgttgggaagggcgatccgtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgcatgtcaggctcccttatacacagccagtctgcaggtcgaatacacttgtaccgaggcaatctcgattccgacaagctaagcttggcactgcgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacc

SEQ ID NO: 61 - DNA, 3325 bp, Artificial Sequence
  Synthetic
gtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcctcagcacaccaccatggcacagattaacaacatggctcaaggcatccagacacttaatcccaattccaacttccataagccccaagtacctaaaagctcaagttttcttgtttttggatctaaaaagctgaaaaattcagcaaattctatgttggttttgaaaaaggattcaatttttatgcaaaagttttgttcctttaggatttcagcatcagtggctacagcctgcatggaggatcttcctgaaaacttggatcaggagtctcttttccaaggattgagagagttcggaatctctacaacttctgcttcttacgctcctcttggtttcggagattaccattggcatatcacaggagatgatggacaaagatggttcgctacagtttctgatttggagcataaggaacattgtggacatggtgctcctgctgctttgagaggtcttagaagagctatggatacagctgttcatttgagagagcaaggaggtcttcctttcgttgttgctcctagaactacatctgatggagcttctttggttcctcttgattctagatacgctttgactgttttcccacatgtttctgctagaccaggagagttcggtcagaaacttacagaaagagagagagatcaagttcttgttcttttggctgagcttcatggacaagctcctcctaagtgtactcctacaactgatatggttccaactggtcttgatggagttcatactgctcttgctgaaccttctggtacttggacaggtggaccattctctgaacctgctagagaacttttggctgaacatgaggctactttgagaggaagaatggctgagttcggtgaattggttgctagagttagaggaagaggtgctcctttggttgttactcatggagagccacatcctggaaatcttatcttgggagaggatggttatgttttggttgattgggatactgttggtcttgctatcccagaaagagatctttctttgatttctgatgatcctgctgctcttgctagatacacagaacttactggacatacacctgatccagctgctttggctctttacagattgagatggtctttgcttgatgttgctgagtttgttgaatggttcagaggagaacatcaaagaacttctgatactgaggctgcttggcagtctttcgctgagactcttgatcatcttaactctgaagttccttcttaatgactagtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 62 - DNA, 1815 bp, Artificial Sequence; identical to SEQ ID NO: 5
  Synthetic

SEQ ID NO: 63 - DNA, 216 bp, Artificial Sequence; identical to SEQ ID NO: 51
  Synthetic

SEQ ID NO: 64 - DNA, 996 bp, Artificial Sequence; identical to SEQ ID NO: 6
  Synthetic

SEQ ID NO: 65 - DNA, 280 bp, Artificial Sequence; identical to SEQ ID NO: 53
  Synthetic

SEQ ID NO: 66 - DNA, 14316 bp, Artificial Sequence
  Synthetic
gcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgagctcgcggccgcgctatgaccatgattacgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcactagtcattaagaaggaacttcagagttaagatgatcaagagtctcagcgaaagactgccaagcagcctcagtatcagaagttctttgatgttctcctctgaaccattcaacaaactcagcaacatcaagcaaagaccatctcaatctgtaaagagccaaagcagctggatcaggtgtatgtccagtaagttctgtgtatctagcaagagcagcaggatcatcagaaatcaaagaaagatctctttctgggatagcaagaccaacagtatcccaatcaaccaaaacataaccatcctctcccaagataagatttccaggatgtggctctccatgagtaacaaccaaaggagcacctcttcctctaactctagcaaccaattcaccgaactcagccattcttcctctcaaagtagcctcatgttcagccaaaagttctctagcaggttcagagaatggtccacctgtccaagtaccagaaggttcagcaagagcagtatgaactccatcaagaccagttggaaccatatcagttgtaggagtacacttaggaggagcttgtccatgaagctcagccaaaagaacaagaacttgatctctctctctttctgtaagtttctgaccgaactctcctggtctagcagaaacatgtgggaaaacagtcaaagcgtatctagaatcaagaggaaccaaagaagctccatcagatgtagttctaggagcaacaacgaaaggaagacctccttgctctctcaaatgaacagctgtatccatagctcttctaagacctctcaaagcagcaggagcaccatgtccacaatgttccttatgctccaaatcagaaactgtagcgaaccatctttgtccatcatctcctgtgatatgccaatggtaatctccgaaaccaagaggagcgtaagaagcagaagttgtagagattccgaactctctcaatccttggaaaagagactcctgatccaagttttcaggaagatcctccatgcaggctgtagccactgatgctgaaatcctaaaggaacaaaacttttgcataaaaattgaatcctttttcaaaaccaacatagaatttgctgaatttttcagctttttagatccaaaaacaagaaaacttgagcttttaggtacttggggcttatggaagttggaattgggattaagtgtctggatgccttgagccatgttgttaatctgtgccatggtggtgtgctgaggacctgcaaagttgaaaaccacaaacgaatcagaatgatgaaatttaacagctatccgactgaattaagtacatctcaagaacagatctgattaatttcagggaaaaaaatacataatcttcggtcaggattaaccaaacaaggcggttgttaatggatctgaattttttttttctttaaaataagaagactgttaacggatctgaaaattttcttaaaaagaaaacaattaacgtaataaagaactacagagaaaaattcaagacgtgttattgtaaaagagaaagaaaagattctgacgatcgatgaaactgtacaaaccctatcttaaactcaacaagctaatcgaaaacatgatcattcacgggactaatgaataacatagacaaaataaaatagacgaaccttgaggggagaggtagggaactgcgcaaagagaatgaagtagtttgttttgaattgaactgtgaagagagacagagggtgtgggcctttttatagctaaaggggtggaatcctctccaatcacattgtgacgcgtgtccttgtgtccctatttggtgcttattacacgggtgatatgtggtgtctgatgcgttttcggagtcctttcttggaaattcaggattgcaatttgcggggtggttagcgggttggtaatgttgacggagtcagacgcgcctacgtcagcttaatctgttacaacggcgttattaaattggaccccacttcacaatttgggccggaaccactgcaagtggagcgttctcagttttgtatgggcccctctatccactacaccaacggttggctcctattttacatgattaagaaatctattgcaaagctgtgatgtttgattttttaatggactaatgaatgattggaaagctttatcatttatttttttaataaggaaatgtcttgtaatcatccacgctttaactatttaaacaaatcatgaaacttaaaggtgacggatttaccaataaaataagggtataggacagcttaattgaaaaaacaaaaatgatatagtaaagaaaacttaagagcgatgggcatccttatcatctaatatgaataagctacaccatctgatctgcagtggacaagttgtcagattatacagttgttgatggctttaagaagcataattgtattattaatatttttatatataagtgcagctctattgtttatactactaatattctcgttttgcgttttatttaataattaattgtatgtactttataaaagaaaaatatcataaaatgagaaaaaaaaacaaaaattgactgttatatttgagtgtttttttacttatatgtattatttatatgtattatgctttaatataatatttttattttactaatatagacatatcaacgcgtgtttcaaactgattttttccctctcatctagatggtgttaatgttccttttatcatcaattaaacttgttgagaaacttatttgttatattaggtatgcaatataatattcgtataaattaaaaaacaaaaaattttgagagttgtttttaaaaattgatatatgggtgagtattaaagtttaaatatgtttaagaaatcattctaagatagaatatgcgcgatgaataaataaaatacctattttttaaaaatatgatatgctattttgtagaatacataaaagatgttttgtatgcgctaacaaaaagggtttttgggaaacaaaacacatataatattatttaagatgtaaaaagtttttccagtttttttttttcaaaacataatacataattcattgatcaatataattacaccttagatgcattcgcgcctgcaggtcatcaaaatatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactaaccaatatttctcgcgatcgcacaccaccatgaagactaatctttttctctttctcatcttttcacttctcctatcattatcctcggccgaattcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagcatgatgagctttaacctagggttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacactcctgcaggatttgaaaaatctcagaattgagattcgggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactgagcgatggtagatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgactcgtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaagctagccaccaccaccaccaccacgtgtgacaagccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagccgtcgtaagcttggcactgcgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacc

SEQ ID NO: 67 - DNA, 1689 bp, Artificial Sequence
  Synthetic
tcatcaaaatatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactaaccaatatttctcatgaagactaatctttttctctttctcatcttttcacttctcctatcattatcctcggccgaattcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagcatgatgagctttaagttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacact

SEQ ID NO: 68 - DNA, 597 bp, Artificial Sequence; identical to SEQ ID NO: 23
  Synthetic

SEQ ID NO: 69 - DNA, 795 bp, Artificial Sequence
  Synthetic
atgaagactaatctttttctctttctcatcttttcacttctcctatcattatcctcggccgaattcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagcatgatgagctttaa

SEQ ID NO: 70 - DNA, 297 bp, Artificial Sequence; identical to SEQ ID NO: 29
  Synthetic

SEQ ID NO: 71 - DNA, 3325 bp, Artificial Sequence; identical to SEQ ID NO: 61
  Synthetic

SEQ ID NO: 72 - DNA, 1815 bp, Artificial Sequence; identical to SEQ ID NO: 5
  Synthetic

SEQ ID NO: 73 - DNA, 216 bp, Artificial Sequence; identical to SEQ ID NO: 51
  Synthetic

SEQ ID NO: 74 - DNA, 996 bp, Artificial Sequence; identical to SEQ ID NO: 6
  Synthetic

SEQ ID NO: 75 - DNA, 276 bp, Artificial Sequence; identical to SEQ ID NO: 9
  Synthetic

SEQ ID NO: 76 - DNA, 24 bp, Artificial Sequence
  Synthetic
tgacaggatatattggcgggtaaa

SEQ ID NO: 77 - DNA, 26 bp, Artificial Sequence
  Synthetic
tggcaggatatattgtggtgtaaaca

SEQ ID NO: 78 - DNA, 977 bp, Artificial Sequence; identical to SEQ ID NO: 3
  Synthetic

SEQ ID NO: 79 - DNA, 182 bp, Artificial Sequence; identical to SEQ ID NO: 41
  Synthetic

SEQ ID NO: 80 - DNA, 795 bp, Artificial Sequence; identical to SEQ ID NO: 42
  Synthetic

SEQ ID NO: 81 - DNA, 12588 bp, Artificial Sequence
  Synthetic
gcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgagctcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcgggtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctagcttggccgggtcccgggctgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatcaaaggtagaagcagaaacttacctagcatggtggccactccaggacgtcgttgtcttacctgcaattcataaaaactacaaaagtcagaaatcaacagtaaaaaaccataacaaatgaaaatccatgcaaagtttcaaaacttctgaatcatgttcactaaaataattgaaccgcatttgacagtttcaaaagtcattatcatgctaattcgtaaattatttcattgtaataaacaaatcatgcttaaccaaacaactaattcattgtgtaataagctaaatgataataagtgaaacagacaagcaataatccaactcagcttatcaatattaacatgaaatttaaaatcaacttaaccacacaagatctcatcaaaccaaatccaacaacacaagagtcataccccaaaagtaatcaataaatcagaataacaaaaaagtacttgttaatcaaaattcagaacagactttcaacatacataatctagatccaattatgaaacatgataaaccagactcatgttattagatacaaaaaaattacattttcacatcaacaaacaaacacagctcaaatttgaagaccattttaattaaataaacgaaaccaaattaaaatcacagtactgtattaacagatccataattaaacaataaaaacatcaaaaactcataacttttcaaacaaaattaaatcatttcaacaacaaaatacacagatctataatttcagattaacaatcggttaaattagctagaattaatcaaatattacaaaatcatatcgattcataattaaaatttctcaaacaataaacagaatcagattaaattcaaaattaaaatttctcatacaaaatttagctcaaacaaattcaatatccaatattacaatcataaatcgattcattcatgattaaatttctcaaacaataaacaaaatcaaaacatgcaaattaaccgaacaaaattatgaaacagattcatttaattcatgtaattgaacaacaaagaaacatagatcgaatcaatgaaacaagaatcggagattgttgttacctcagctgaatcgaaatctagggttgggaactctcagccgcagagagagacggagtgatatttgagagtgaaagaaatgagtgagaatgaaaaaacctagggttacaatgtttttatagggtcccagatatttttatgttccaaaagtgtccttgctgaattttggatttccgaaaataccctaatgtgaacgttggagagtggcggatggaatatgcttttgtgagttttttaggtaaggatggaataagaaagaaggttaggttaaggtattgtgacgacatcgtttcatttaggtttgtaatgtctgtgacattgtttttttgggcttttctttgagtgtggcccaagtgaaatattgggcaaaaccaaacatgaaggtttcttcttcaaaaaattattaattaatagacatataatgtcaaataggtcataatgtccgacatacaatgtcaattcatttcgaaaaaagttagtccggtaagtttttaggtcatgctaattgtgatgtcattaaagctagaggtgtatgacaagataatgttgtaagagtctaagtcacgctaacgagtgagatccaaggatgttagttaagaaaaggaaaaatgaaaatttggtattggaaataacaaatttcgaactttcaaaaagttgagtaagattgaatacaccactttttatgacaggttttttaaaaaataagtctttagcaagtgtcctaaatgcgttttttaacatttccccaagtttttttttaataacaatttagtcatttaagtttgtaaacatttacattgttgtttttttaaccatattttgttagaaaaacaaaaagcttatgtgtctgttgaatactcgatgtggcacgaaggttgcgcacgaattcgattctaggtacctcgcgaatgcatctaaggtgtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatggagaataccggaggtgtgagaaaaggcgcatggacttacaaggaagatgagctactcaaggcttgcattaacacgtacggtgaaggaaaatggaatttagttcctcagagatctggattgaatagatgcagaaaaagttgtagattgaggtggttaaattacttaagccccaacatcaacagaggaagattttctgaggatgaagaagatttgatcctaaggttacacaaactactaggaaatagatggtcattgattgctggaaggcttccgggtagaacagctaatgatgtgaagaactattggcacacaaatttggcaaagaaagtggtttcagaaaaggaagaagagaaagaaaacgataaacctaaggaaaccatgaaagctcatgaagttattaaacctcgtcctataactttgtcaagtcattcaaattggttgaaggggaaaaatagtattcctagagatcttgattactcagagaatatggcttcaaatcaaattggtagagagtgtgcttctacttcaaaaccagatctaggtaatgccccaataccatgtgaaatgtggtgtgacagtttgtggaacttgggagaacatgtagacagtgagaaaattgggtcatgctcctcattacaagaggagaacttaatggagtttccaaatgttgatgatgactccttttgggatttcaacctttgtgatttgaattctctttgggatctaccttgacccgggagacctttaaacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagctgaggtcgactgcagaggcctgcatgcaagagcgcgaccttcccgtgggacgtgcaagctaagcttggcactgcgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacc

SEQ ID NO: 82 - DNA, 2855 bp, Artificial Sequence
  Synthetic
gtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatggagaataccggaggtgtgagaaaaggcgcatggacttacaaggaagatgagctactcaaggcttgcattaacacgtacggtgaaggaaaatggaatttagttcctcagagatctggattgaatagatgcagaaaaagttgtagattgaggtggttaaattacttaagccccaacatcaacagaggaagattttctgaggatgaagaagatttgatcctaaggttacacaaactactaggaaatagatggtcattgattgctggaaggcttccgggtagaacagctaatgatgtgaagaactattggcacacaaatttggcaaagaaagtggtttcagaaaaggaagaagagaaagaaaacgataaacctaaggaaaccatgaaagctcatgaagttattaaacctcgtcctataactttgtcaagtcattcaaattggttgaaggggaaaaatagtattcctagagatcttgattactcagagaatatggcttcaaatcaaattggtagagagtgtgcttctacttcaaaaccagatctaggtaatgccccaataccatgtgaaatgtggtgtgacagtttgtggaacttgggagaacatgtagacagtgagaaaattgggtcatgctcctcattacaagaggagaacttaatggagtttccaaatgttgatgatgactccttttgggatttcaacctttgtgatttgaattctctttgggatctaccttgacccgggagacctttaaacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 83 - DNA, 1815 bp, Artificial Sequence; identical to SEQ ID NO: 5
  Synthetic

SEQ ID NO: 84 - DNA, 735 bp, Artificial Sequence
  Synthetic
atggagaataccggaggtgtgagaaaaggcgcatggacttacaaggaagatgagctactcaaggcttgcattaacacgtacggtgaaggaaaatggaatttagttcctcagagatctggattgaatagatgcagaaaaagttgtagattgaggtggttaaattacttaagccccaacatcaacagaggaagattttctgaggatgaagaagatttgatcctaaggttacacaaactactaggaaatagatggtcattgattgctggaaggcttccgggtagaacagctaatgatgtgaagaactattggcacacaaatttggcaaagaaagtggtttcagaaaaggaagaagagaaagaaaacgataaacctaaggaaaccatgaaagctcatgaagttattaaacctcgtcctataactttgtcaagtcattcaaattggttgaaggggaaaaatagtattcctagagatcttgattactcagagaatatggcttcaaatcaaattggtagagagtgtgcttctacttcaaaaccagatctaggtaatgccccaataccatgtgaaatgtggtgtgacagtttgtggaacttgggagaacatgtagacagtgagaaaattgggtcatgctcctcattacaagaggagaacttaatggagtttccaaatgttgatgatgactccttttgggatttcaacctttgtgatttgaattctctttgggatctaccttga

SEQ ID NO: 85 - DNA, 279 bp, Artificial Sequence
  Synthetic
cgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 86 - DNA, 1661 bp, Artificial Sequence; identical to SEQ ID NO: 30
  Synthetic

SEQ ID NO: 87 - DNA, 1887 bp, Artificial Sequence
  Synthetic
cgcaaccttcgtgccacatcgagtattcaacagacacataagctttttgtttttctaacaaaatatggttaaaaaaacaacaatgtaaatgtttacaaacttaaatgactaaattgttattaaaaaaaaacttggggaaatgttaaaaaacgcatttaggacacttgctaaagacttattttttaaaaaacctgtcataaaaagtggtgtattcaatcttactcaactttttgaaagttcgaaatttgttatttccaataccaaattttcatttttccttttcttaactaacatccttggatctcactcgttagcgtgacttagactcttacaacattatcttgtcatacacctctagctttaatgacatcacaattagcatgacctaaaaacttaccggactaacttttttcgaaatgaattgacattgtatgtcggacattatgacctatttgacattatatgtctattaattaataattttttgaagaagaaaccttcatgtttggttttgcccaatatttcacttgggccacactcaaagaaaagcccaaaaaaacaatgtcacagacattacaaacctaaatgaaacgatgtcgtcacaataccttaacctaaccttctttcttattccatccttacctaaaaaactcacaaaagcatattccatccgccactctccaacgttcacattagggtattttcggaaatccaaaattcagcaaggacacttttggaacataaaaatatctgggaccctataaaaacattgtaaccctaggttttttcattctcactcatttctttcactctcaaatatcactccgtctctctctgcggctgagagttcccaaccctagatttcgattcagctgaggtaacaacaatctccgattcttgtttcattgattcgatctatgtttctttgttgttcaattacatgaattaaatgaatctgtttcataattttgttcggttaatttgcatgttttgattttgtttattgtttgagaaatttaatcatgaatgaatcgatttatgattgtaatattggatattgaatttgtttgagctaaattttgtatgagaaattttaattttgaatttaatctgattctgtttattgtttgagaaattttaattatgaatcgatatgattttgtaatatttgattaattctagctaatttaaccgattgttaatctgaaattatagatctgtgtattttgttgttgaaatgatttaattttgtttgaaaagttatgagtttttgatgtttttattgtttaattatggatctgttaatacagtactgtgattttaatttggtttcgtttatttaattaaaatggtcttcaaatttgagctgtgtttgtttgttgatgtgaaaatgtaatttttttgtatctaataacatgagtctggtttatcatgtttcataattggatctagattatgtatgttgaaagtctgttctgaattttgattaacaagtacttttttgttattctgatttattgattacttttggggtatgactcttgtgttgttggatttggtttgatgagatcttgtgtggttaagttgattttaaatttcatgttaatattgataagctgagttggattattgcttgtctgtttcacttattatcatttagcttattacacaatgaattagttgtttggttaagcatgatttgtttattacaatgaaataatttacgaattagcatgataatgacttttgaaactgtcaaatgcggttcaattattttagtgaacatgattcagaagttttgaaactttgcatggattttcatttgttatggttttttactgttgatttctgacttttgtagtttttatgaattgcaggtaagacaac

SEQ ID NO: 88 - DNA, 187 bp, Artificial Sequence; identical to SEQ ID NO: 32
  Synthetic

SEQ ID NO: 89 - DNA, 825 bp, Artificial Sequence; identical to SEQ ID NO: 33
  Synthetic

SEQ ID NO: 90 - DNA, 160 bp, Artificial Sequence; identical to SEQ ID NO: 34
  Synthetic

SEQ ID NO: 91 - DNA, 24 bp, Artificial Sequence
  Synthetic
tgacaggatatattggcgggtaaa

SEQ ID NO: 92 - DNA, 26 bp, Artificial Sequence
  Synthetic
tggcaggatatattgtggtgtaaaca

SEQ ID NO: 93 - DNA, 977 bp, Artificial Sequence; identical to SEQ ID NO: 3
  Synthetic

SEQ ID NO: 94 - DNA, 182 bp, Artificial Sequence; identical to SEQ ID NO: 41
  Synthetic

SEQ ID NO: 95 - DNA, 795 bp, Artificial Sequence; identical to SEQ ID NO: 42
  Synthetic